(19) United States
(12) Patent Application Publication
(10) Pub. No.: US 2022/0359084 A1
(43) Pub. Date: Nov. 10, 2022

(54) Methods of Treatments Based Upon Molecular Characterization of Breast Cancer

(71) Applicant: The Board of Trustees of the Leland Stanford Junior University, Stanford, CA (US)

(72) Inventors: Christina Curtis, Stanford, CA (US); Jose A. Seoane Fernandez, L´Hospitalet de Llobregat (ES)

(73) Assignee: The Board of Trustees of the Leland Stanford Junior University, Stanford, CA (US)

(21) Appl. No.: 17/753,871

(22) PCT Filed: Sep. 16, 2020
(86) PCT No.: PCT/US2020/051130

§ 371 Date: Mar. 16, 2022

Related U.S. Application Data
(60) Provisional application No. 62/901,175, filed on Sep. 16, 2019.

(51) Int. Cl. G16H 50/30; G16H 50/20; G16B 20/00; A61K 45/06

(52) U.S. Cl. CPC G16H 50/30; G16H 50/20; G16B 20/00; A61K 45/06

(57) ABSTRACT

Stratification of risk and methods of treatment based on a breast cancer's molecular profile are provided. Copy number aberrations of various genomic loci and expression levels of various genes are used to molecularly subtype patients and in some instances to determine a breast cancer's aggressiveness and risk of relapse. Breast cancers having a particular molecular subtype with an associated risk of relapse can be stratified and therapeutically targeted.

### CROSS REFERENCE TO RELATED APPLICATIONS

This application claims priority to U.S. Provisional Application Ser. No. 62/901,175, entitled “Methods of Treatments Based Upon Molecular Characterization of Breast Cancer” by Christina Curtis et al., filed Sep. 16, 2019, which is incorporated herein by reference in its entirety.

### TECHNICAL FIELD

The invention is generally directed to methods of diagnostics and treatments based upon a molecular characterization of an individual's breast cancer, and more specifically to treatments based upon molecular diagnostics indicative of aggressiveness, relapse risk of breast cancer, or molecular subtype.

### BACKGROUND

Breast cancer is the most frequent cancer diagnosis and cause of cancer death in women worldwide with 1.4 million diagnoses and 500,000 deaths annually. Survival rates have dramatically improved due to new treatments but a sizable minority of patients suffer from an aggressive form of cancer and/or experience a relapse, which may be incurable. Most cancer registries do not record recurrence information and the rates of relapse are poorly characterized. Analysis of retrospective cohorts and clinical trials have provided some insights into patterns of recurrence. For example, some estrogen receptor-positive (ER+) tumors continue to recur well past five years with a higher rate of bone metastasis, while estrogen receptor-negative (ER−) tumors recur more quickly and have higher rates of visceral metastases. However, methods to reliably stratify risk of relapse are lacking as are therapeutic approaches for early stage breast cancer patients who are at high risk of relapse or who have already recurred on the basis of their tumor molecular profile.

### SUMMARY

Various embodiments are directed towards methods treatments for breast cancer based on its molecular characterization. In various embodiments, the molecular subtype of a breast cancer is determined based on its genetics. In various embodiments, a molecular subtype is indicative breast cancer aggressiveness and risk of relapse. In various embodiments, a molecular subtype is indicative of the molecular pathology of a breast cancer. In various embodiments, a breast cancer is treated based upon aggressiveness, risk of relapse, and molecular drivers as determined by its molecular subtype.

In an embodiment, an individual having breast cancer is treated. A breast cancer of an individual is stratified utilizing a risk stratification model into a high risk of recurrence subgroup. The risk stratification model is a statistical model that incorporates features derived from integrative subtype clusters that are delineated by a molecular pathology. The individual is treated to reduce the risk of recurrence by administering a prolonged treatment regimen that includes chemotherapy, endocrine therapy, targeted therapy, or health professional surveillance.

In another embodiment, the risk stratification model utilizes a multi-state semi-markov Model, a Cox Proportional Hazards model, a shrinkage based method, a tree based method, a Bayesian method, a kernel based method, or a neural network.

In yet another embodiment, the integrated subtype cluster features are membership to a given cluster or the posterior probability of membership to a given cluster.

In a further embodiment, the integrative subtype clusters are determined by the IntClust classification model that incorporates molecular data as features.

In still yet another embodiment, the molecular data is obtained by microarray based gene expression, microarray/SNP array based copy number inference, RNA-sequencing, targeted (capture) RNA-sequencing, exome sequencing, whole genome sequencing (WES/WGS), targeted (panel) sequencing, Nanostring nCounter for gene expression, Nanostring nCounter for copy number inference, Nanostring digital spatial profiler measurement of protein, Nanostring digital spatial profiler measurement of protein gene expression in situ, DNA-ISH, RNA-ISH, RNAScope, DNA Methylation assays, or ATAC-seq.

In yet a further embodiment, the molecular data is derived utilizing a gene panel.

In an even further embodiment, the gene panel is one of: Foundation Medicine CDx, Memorial Sloan Kettering Cancer Center Integrated Mutation Profiling of Actionable Cancer Targets (MSK-IMPACT), Stanford Tumor Actionable Mutation Panel (STAMP), or UCSF500 Cancer Gene Panel.

In yet an even further embodiment, the risk stratification model utilizes clinical data, such as age, cancer stage, number of tumor positive lymph nodes, size of tumor, grade of tumor, surgery performed, treatment performed, or basic molecular identities.

In still yet an even further embodiment, the risk stratification model utilizes the CTS5 algorithm.

In still yet an even further embodiment, the risk stratification model incorporates Oncotype DX, Prosigna PAM50, Prosigna ROR, MammaPrint, EndoPredict or Breast Cancer Index (BC).

In still yet an even further embodiment, the prolonged treatment regimen includes adjuvant chemotherapy.

In still yet an even further embodiment, the prolonged treatment regimen includes treatment beyond the standard course of treatment.

In an embodiment, an individual having breast cancer is treated. A breast cancer of an individual is stratified utilizing a risk stratification model into a lower risk of recurrence subgroup. The risk stratification model is a statistical model that incorporates features derived from integrative subtype clusters that are delineated by a molecular pathology. The individual is treated to reduce the harmful effects of chemotherapy by administering a treatment regimen that includes surgery or endocrine therapy, but not chemotherapy.

In another embodiment, the risk stratification model utilizes a multi-state semi-markov Model, a Cox Proportional Hazards model, a shrinkage based method, a tree based method, a Bayesian method, a kernel based method, or a neural network.

In yet another embodiment, the integrated subtype cluster features are membership to a given cluster or the posterior probability of membership to a given cluster.

In a further embodiment, the integrative subtype clusters are determined by the IntClust classification model that incorporates molecular data as features.

In still yet another embodiment, the molecular data is obtained by microarray based gene expression, microarray/SNP array based copy number inference, RNA-sequencing, targeted (capture) RNA-sequencing, exome sequencing, whole genome sequencing (WES/WGS), targeted (panel) sequencing, Nanostring nCounter for gene expression, Nanostring nCounter for copy number inference, Nanostring digital spatial profiler measurement of protein, Nanostring digital spatial profiler measurement of protein gene expression in situ, DNA-ISH, RNA-ISH, RNAScope, DNA Methylation assays, or ATAC-seq.

In yet a further embodiment, the molecular data is derived utilizing a gene panel.

In an even further embodiment, the gene panel is one of: Foundation Medicine CDx, Memorial Sloan Kettering Cancer Center Integrated Mutation Profiling of Actionable Cancer Targets (MSK-IMPACT), Stanford Tumor Actionable Mutation Panel (STAMP), or UCSF500 Cancer Gene Panel.

In yet an even further embodiment, the risk stratification model utilizes clinical data, such as age, cancer stage, number of tumor positive lymph nodes, size of tumor, grade of tumor, surgery performed, treatment performed, or basic molecular identities.

In still yet an even further embodiment, the risk stratification model utilizes the CTS5 algorithm.

In still yet an even further embodiment, the risk stratification model incorporates Oncotype DX, Prosigna PAM50, Prosigna ROR, MammaPrint, EndoPredict or Breast Cancer Index (BC).

In still yet an even further embodiment, the treatment regimen includes adjuvant endocrine therapy.

In an embodiment, an individual having breast cancer is treated. The results an assay is determined, classifying an individual's breast cancer into an integrated cluster (IntClust) subgroup. The results indicate that the breast cancer is classified into one of: IntClust1, IntClust2, IntClust6, or IntClust9. The individual is treated with a prolonged treatment regimen that includes chemotherapy, endocrine therapy, targeted therapy, and health professional surveillance.

In another embodiment, the classification of the individual's breast cancer is performed utilizing a molecular class prediction tool.

In yet another embodiment, the molecular class prediction tool utilizes a shrinkage based method, logistic regression, a support vector machine with a linear kernel, a support vector machine with a gaussian kernel, or a neural network.

In a further embodiment, the molecular class prediction tool incorporates molecular data as features.

In still yet another embodiment, the molecular data features are copy number features, gene expression features, genomic methylation features, or occupancy features derived from DNA or RNA analysis of the individual's breast cancer.

In yet a further embodiment, the molecular data is obtained by microarray based gene expression, microarray/SNP array based copy number inference, RNA-sequencing, targeted (capture) RNA-sequencing, exome sequencing, whole genome sequencing (WES/WGS), targeted (panel) sequencing, Nanostring nCounter for gene expression, Nanostring nCounter for copy number inference, Nanostring digital spatial profiler measurement of protein, Nanostring digital spatial profiler measurement of protein gene expression in situ, DNA-ISH, RNA-ISH, RNAScope, DNA Methylation assays, or ATAC-seq.

In an even further embodiment, the molecular data is derived utilizing a gene panel.

In yet an even further embodiment, the gene panel is Foundation Medicine CDx, Memorial Sloan Kettering Cancer Center Integrated Mutation Profiling of Actionable Cancer Targets (MSK-IMPACT), Stanford Tumor Actionable Mutation Panel (STAMP), or UCSF500 Cancer Gene Panel.

In still yet an even further embodiment, the breast cancer the individual is administered adjuvant chemotherapy.

In still yet an even further embodiment, the breast cancer the individual is administered extended endocrine therapy.

In still yet an even further embodiment, the endocrine therapy comprises administering a selective estrogen receptor modulator, a selective estrogen receptor degrader, an aromatase inhibitor, or PROTAC ARV-471.

In still yet an even further embodiment, the selective estrogen receptor modulator is tamoxifen, toremifene, raloxifene, ospemifene, or bazedoxifene.

In still yet an even further embodiment, the selective estrogen receptor degrader is fulvestrant, brilanestrant (GDC-0810), elacestrant, GDC-9545, SAR439859 (SERD '859), RG6171, or AZD9833.

In still yet an even further embodiment, the aromatase inhibitor is anastrozole, exemestane, letrozole, vorozole, formestane, or fadrozole.

In still yet an even further embodiment, the breast cancer is classified into IntClust1 and the individual is administered an mTOR pathway antagonist, an AKT1 antagonist, an AKT1/RPS6KB1 antagonist, an RPS6KB1 antagonist, a PI3K antagonist, an elF4A antagonist, or an elF4E antagonist.

In still yet an even further embodiment, the breast cancer is classified into IntClust2 and the individual is administered a CDK4/6 antagonist, an FGFR pathway antagonist, a PARP antagonist, a homologous recombination deficiency (HRD) targeted therapy, a PAK1 antagonist, an elF4A antagonist, or elF4E antagonist.

In still yet an even further embodiment, the breast cancer is classified into IntClust6 and the individual is administered an FGFR pathway antagonist, an elF4A antagonists, or an elF4E antagonist.

In still yet an even further embodiment, the breast cancer is classified into IntClust9 and the individual is administered a selective estrogen receptor degrader, an SRC3 antagonist, a MYC antagonist, a BET bromodomain antagonist, an elF4A antagonist, or an elF4E antagonist.

In an embodiment, an individual having breast cancer is treated. An oncogenic pathology of an individual's cancer is classified. The oncogenic pathology indicates mTOR pathway. The individual is administered an mTOR antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the mTOR antagonist is everolimus, temsirolimus, sirolimus, or rapamycin.

In an embodiment, an individual having breast cancer is treated. An oncogenic pathology of an individual's cancer is classified. The oncogenic pathology indicates AKT1. The individual is administered an AKT1 antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the AKT1 antagonist is ipatasertib, or capivasertib (AZD5363).

In an embodiment, an individual having breast cancer is treated. An oncogenic pathology of an individual's cancer is classified. The oncogenic pathology indicates AKT1/RPS6KB1. The individual is administered an AKT1/RPS6KB1 antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the AKT1/RPS6KB1antagonist is M2698.

In an embodiment, an individual having breast cancer is treated. An oncogenic pathology of an individual's cancer is classified. The oncogenic pathology indicates RPS6KB1. The individual is administered an RPS6KB1 antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the RPS6KB1 antagonist is LY2584702.

In an embodiment, an individual having breast cancer is treated. An oncogenic pathology of an individual's cancer is classified. The oncogenic pathology indicates PI3K. The individual is administered an PI3K antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the PI3K antagonist is alpelisib, buparlisib (BKM120), or pictilisib (GDC-0941).

In an embodiment, an individual having breast cancer is treated. An oncogenic pathology of an individual's cancer is classified. The oncogenic pathology indicates CDK4/6. The individual is administered an CDK4/6 antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the CDK4/6 antagonist is palbociclib, ribociclib, or abemaciclib.

In an embodiment, an individual having breast cancer is treated. An oncogenic pathology of an individual's cancer is classified. The oncogenic pathology indicates FGFR pathway. The individual is administered an FGFR pathway antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the FGFR pathway antagonist is lucitanib, dovitinib, AZD4547, erdafitinib, infigratinib (BGJ398), BAY-1163877, or ponatinib.

In an embodiment, an individual having breast cancer is treated. An oncogenic pathology of an individual's cancer is classified. The oncogenic pathology indicates SRC3. The individual is administered an SRC3 antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the SRC3 antagonist is SI-2.

In an embodiment, an individual having breast cancer is treated. An oncogenic pathology of an individual's cancer is classified. The oncogenic pathology indicates MYC. The individual is administered a MYC antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the MYC antagonist is omomyc.

In an embodiment, an individual having breast cancer is treated. An oncogenic pathology of an individual's cancer is classified. The oncogenic pathology indicates BET bromodomain. The individual is administered an BET bromodomain antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the BET bromodomain antagonist is JQ1 or PROTAC ARV-771.

In an embodiment, an individual having breast cancer is treated. An oncogenic pathology of an individual's cancer is classified. The oncogenic pathology indicates elF4A. The individual is administered an elF4A antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the elF4A antagonist is zotatifin.

In an embodiment, an individual having breast cancer is treated. An oncogenic pathology of an individual's cancer is classified. The oncogenic pathology indicates v. The individual is administered an elF4E antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the elF4E antagonist is rapamycin, a rapamycin analogue, ribavirin, or AZD8055.

In an embodiment, an individual having breast cancer is treated. An oncogenic pathology of an individual's cancer is classified. The oncogenic pathology indicates PARP. The individual is administered a PARP antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the PARP antagonist is niraparib or olaparib.

In an embodiment, an individual having breast cancer is treated. An oncogenic pathology of an individual's cancer is classified. The oncogenic pathology indicates PAK1. The individual is administered a PAK1 antagonist.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular prediction tool also utilizes copy number features, gene expression features, genomic methylation features, or nucleosome occupancy features derived from DNA or RNA analysis of the individual's breast cancer.

In yet another embodiment, the PAK1 antagonist is IPA3.

In an embodiment, drug compounds are assessed utilizing breast cancer patient derived organoids. Cancer cells are extracted from one or more patients. The oncogenic pathology of each patient's cancer is classified into a molecular pathology subgroup. A panel of patient derived organoid lines is developed utilizing the extracted cancer cells. Each patient derived organoid line of the panel is within the same molecular pathology subgroup. A plurality of drug compounds is administered on the panel of patient derived organoid lines to assess the toxicity of each drug compound.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular class prediction tool also utilizes copy number features, gene expression features, genomic methylation features, or nucleosome occupancy features derived from DNA or RNA analysis of the patient's breast cancer or of the patient derived organoid line.

In yet another embodiment, the molecular pathology subgroup is an integrated cluster subgroup.

In a further embodiment, compound concentration is assessed.

In still yet another embodiment, compound toxicity on healthy cells is assessed.

In an embodiment, drug compounds are assessed for a personalized treatment utilizing breast cancer patient derived organoids. Cancer cells are extracted from a patient. The oncogenic pathology the patient's cancer is classified into a molecular pathology subgroup. One or more patient derived organoid lines is developed using the extracted cancer cells. A plurality of drug compounds is administered on the one or more patient derived organoid lines to assess the toxicity of each drug compound. The drug compounds to be administered are candidate compounds associated with the molecular pathology subgroup.

In another embodiment, the oncogenic pathology is classified utilizing a molecular class prediction tool that utilizes a shrinkage based method, logistic regression, a support vector machine with a linear kernel, a support vector machine with a gaussian kernel, or a neural network. The molecular class prediction tool also utilizes copy number features, gene expression features, genomic methylation features, or nucleosome occupancy features derived from DNA or RNA analysis of the patient's breast cancer or of the patient derived organoid line.

In yet another embodiment, the molecular pathology subgroup is an integrated cluster subgroup.

In a further embodiment, compound concentration is assessed.

In still yet another embodiment, compound toxicity on healthy cells is assessed.

In yet a further embodiment, at least one combination of the drug compounds is assessed.

In an even further embodiment, the patient is administered a drug compound of the plurality of drug compounds based on the drug compound's toxicity on the one or more patient derived organoid lines.

In yet an even further embodiment, the drug compound is administered as an adjuvant therapy.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIGS. 1A to 1F provides a list of genomic assays for breast cancer characterization in accordance with the prior art.

FIGS. 2A and 2B provide a map of chromosomal copy number aberrations and their prevalence across Integrative Clusters, generated in the prior art and utilized as reference.

FIGS. 3A and 3B provide bar graphs indicating the percent of breast cancers within a high risk integrative cluster experiencing a copy number gain or amplification in the genes listed, generated in the prior art and utilized as reference.

FIG. 4 provides probabilities of relapse for the subgroups of the Integrative Cluster system, generated in the prior art and utilized as reference.

FIG. 5 provides probabilities of relapse over time for the ER+ subgroups of the Integrative Cluster system, utilized in accordance with various embodiments of the invention.

FIG. 6 provides bar graphs indicating the percent of breast cancers divided into integrative cluster subgroups experiencing a copy number gain of particular genes, utilized in accordance with various embodiments of the invention.

FIG. 7 provides a flow diagram of a method to treat a breast cancer based upon classification into a molecular subgroup in accordance with various embodiments of the invention.

FIG. 8 provides a flow chart of the METABRIC cohort clinical characteristics and inclusion analysis, generated in the prior art and utilized as reference.

FIG. 9 provides a flow chart of the external validation metacohort clinical characteristics and inclusion analysis, generated in the prior art and utilized as reference.

FIGS. 10 and 11 provide data graphs depicting cumulative incidence of death for ER+ and ER− patients, generated in the prior art and utilized as reference.

FIG. 12 provides a data chart detailing the average age at onset of breast cancer in ER+ and ER− patients, generated in the prior art and utilized as reference.

FIG. 13 provides a graphical representation of a multistate Markov model of breast cancer progression, generated in the prior art and utilized as reference.

FIG. 14 provides a data chart depicting prognostic values of clinical covariates at different disease states, generated in the prior art and utilized as reference.

FIG. 15 provides data charts depicting the internal validation of the global prediction of the models on all transitions using bootstrap, generated in the prior art and utilized as reference.

FIG. 16 provides a scatterplot of predictions of disease-specific death risk computed by two computational models based on ER status at ten years, demonstrating strong concordance for a simple model, generated in the prior art and utilized as reference.

FIG. 17 provides concordance c-indexes of prediction of risks of distant relapse (dr), disease-specific death (ds), death (os) and relapse (r), generated in the prior art and utilized as reference.

FIGS. 18 and 19 provide data charts depicting probability of relapse of various subgroups over time, generated in the prior art and utilized as reference.

FIG. 20 provides data charts depicting associations between probabilities of distant relapse after 10 year of loco-regional relapse and several clinic-pathological and molecular features, generated in the prior art and utilized as reference.

FIGS. 21 to 26 provide data charts depicting average probability of relapse or cancer-related death after surgery in various subgroups over time, generated in the prior art and utilized as reference.

FIG. 27 provides a data graph depicting the evaluation of predictive utility of a standard clinical model relative to a model incorporating the integrative cluster subtypes, generated in the prior art and utilized as reference.

FIG. 28 provides a data graph depicting probabilities of distant relapse or breast cancer death among ER+/Her2− patients who were relapse free at 5 years post diagnosis, generated in the prior art and utilized as reference.

FIG. 29 provides a data graph depicting probabilities of distant relapse or breast-specific death for individual average ER+/HER2− patients in the four late-relapsing subgroups relative to IntClust3 for patients who were relapse free five years post diagnosis, generated in the prior art and utilized as reference.

FIG. 30 provides receiver operating characteristic and precision recall curves of various computational models utilizing whole genome copy number data, utilized in accordance with various embodiments of the invention.

FIGS. 31A and 31B each provide results of stratifying risk of breast cancers utilizing various sequencing panels, utilized in accordance with various embodiments of the invention.

FIG. 32A provides sensitivity and specificity results of a classifier to predict high risk IntClust subgroups using the Foundation Medicine targeted sequencing gene panel, generated in accordance with various embodiments of the invention.

FIG. 32B provides sensitivity and specificity results of a classifier to predict high risk IntClust subgroups using the MSK-IMPACT targeted sequencing gene panel, generated in accordance with various embodiments of the invention.

FIG. 32C provides distribution of IntClust subgroups predicted using the MSK-IMPACT targeted sequencing gene panel, generated in accordance with various embodiments of the invention.

FIG. 33 provides C-index scores of various diagnostic tests at predicting recurrence of breast cancer, utilized in accordance with various embodiments of the invention.

FIGS. 34 to 37 each provide hazard ratio scores of various diagnostic tests at predicting recurrence of breast cancer, utilized in accordance with various embodiments of the invention.

FIG. 38 provides results of stratifying breast cancer risk of recurrence by various diagnostic tests, utilized in accordance with various embodiments of the invention.

FIGS. 39 to 43 each provide results of stratifying breast cancer risk of recurrence utilizing the IntClust classification system in combination with various diagnostic tests, utilized in accordance with various embodiments of the invention.

FIGS. 44 to 51 each provide probabilities of progression free survival of various high-risk oncogenic molecular subgroups in various forms treatments, including chemotherapy, targeted (molecular) therapy, or endocrine therapy, utilized in accordance with various embodiments of the invention.

FIGS. 52A and 52B provide viability curves of patient derived organoids derived from patient 19006, generated in accordance with various embodiments of the invention.

FIGS. 53A and 53B provide viability curves of patient derived organoids derived from patient 19004, generated in accordance with various embodiments of the invention.

### DETAILED DESCRIPTION

Turning now to the drawings and data, systems, kits, and methods of determining breast cancer aggressiveness and potential for relapse and treating breast cancer based upon the cancer's molecular pathology are provided. Many embodiments are directed to determining a breast cancer's aggressiveness and potential for relapse utilizing a diagnostic assay. Many embodiments are directed to determining a breast cancer's molecular pathology utilizing a diagnostic assay. In a number of embodiments, a determination of a breast cancer's aggressiveness and potential for relapse and/or molecular pathology is then used to determine a treatment option, and to treat that neoplasm accordingly. In various embodiments, somatic copy-number or transcript-expression data provide an indication of breast cancer molecular subtype and thus provide a means of determining appropriate treatment. In some embodiments, gene copy number changes or aberrant expression of molecular drivers of cancer progression are determined as basis of a cancer's pathology. In accordance with multiple embodiments, breast cancers exhibiting particular molecular pathologies indicating high aggression and high potential for relapse are treated aggressively with an appropriate therapy, such as adjuvant chemotherapy, targeted therapy, and/or prolonged hormone/endocrine therapy. Furthermore, in several embodiments, individuals with cancer that have high potential for relapse are closely and repeatedly monitored for an extended period of time after a surgical and/or chemotherapy treatment, including treatments that reduce the cancer to undetectable levels. In various embodiments, cancers having a particular molecular pathology are treated with therapies that are directed at the genes that classify the molecular pathology by targeting the gene, the gene product, and/or the molecular pathway involving the gene. In accordance with many embodiments, breast cancer exhibiting a molecular pathology indicative of low aggression and recurrence are treated appropriately, which may be only endocrine therapy or less aggressive chemotherapy.

A number of embodiments are directed to determining an individual's molecular pathology. In many embodiments, copy number aberrations (CNAs) are assessed from an individual's DNA and/or RNA, which can be used to classify an individual's cancer. CNAs are to be understood as amplification (e.g., duplication) and/or reduction (e.g., deletion) of a set of genomic loci within the genome of a cancer. In some embodiments, a cancer is classified by copy number aberrations that include a set of one or more molecular drivers (i.e., genes classified to be at least partially pathogenic in tumorigenesis). Various embodiments utilize the integrative cluster (IntClust) classification to determine a set of molecular drivers that describe the pathogenesis of a breast cancer. For more on the IntClust classification system, see C. Curtis, et al., Nature 486, 346-52 (2012) and H. R. Ali, et al., Genome Biol. 15, 431 (2014), the disclosures of which are each herein incorporated by reference. In many embodiments, the risk of relapse is determined by a risk classifier.

Based on recent discoveries, the connection between the molecular pathology and cancer progression, including the potential for reoccurrence, is now appreciated, indicating courses of treatment and surveillance. Accordingly, various embodiments are directed to classifying breast cancer into an IntClust subgroup and/or risk subgroup to determine a treatment regimen that is tailored for a particular breast cancer. In addition, a number of tools and kits are described to classify a breast cancer into an IntClust and/or risk subgroup.

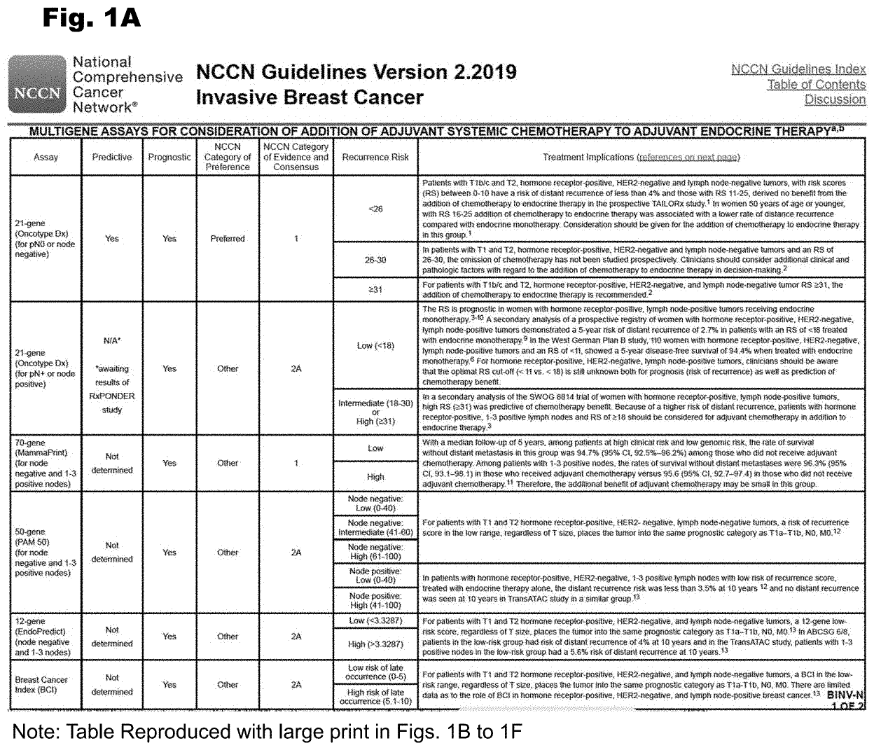

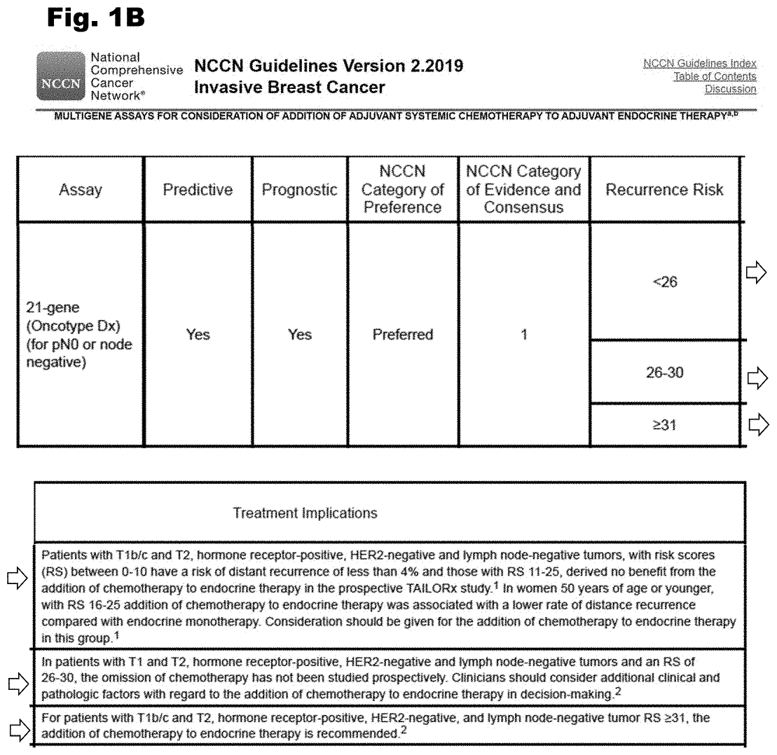

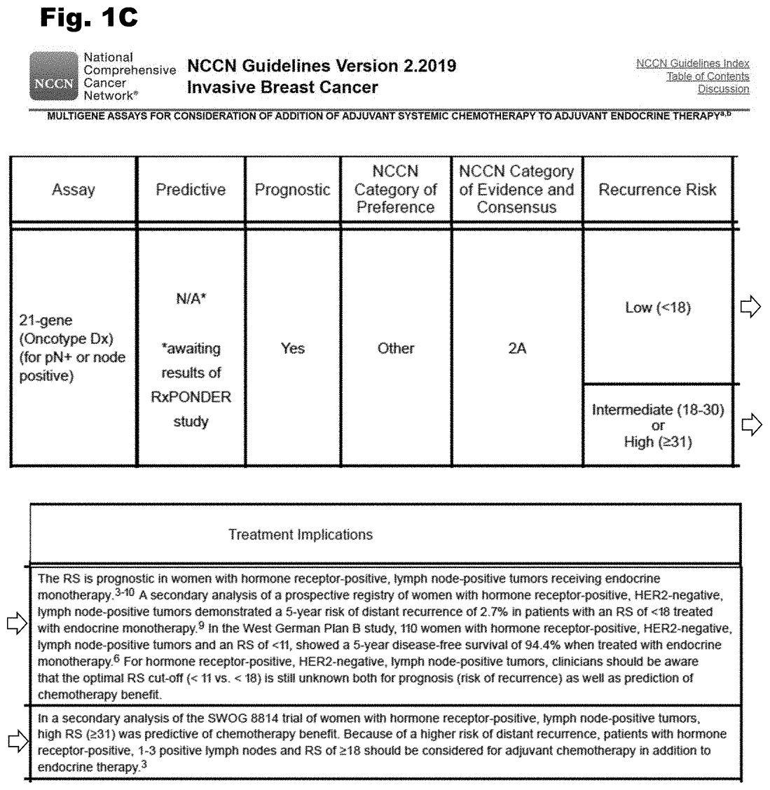

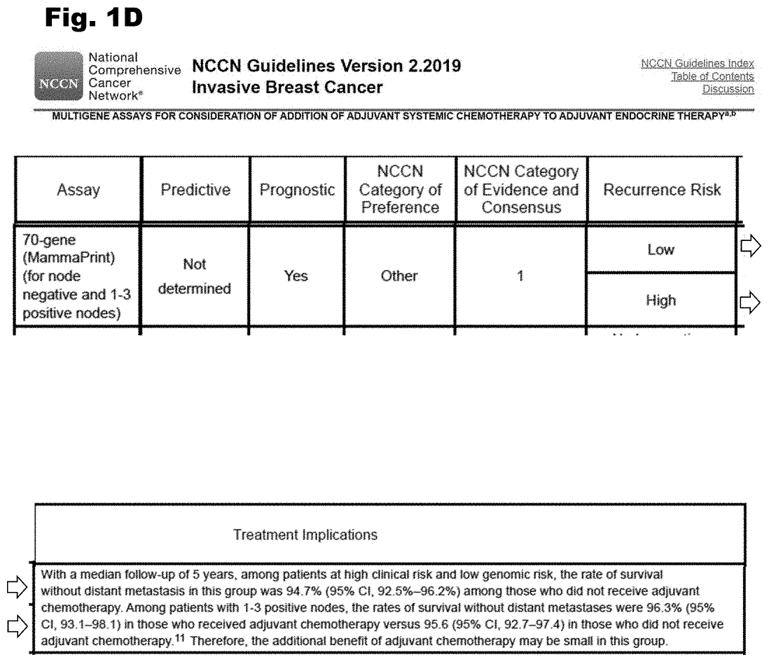

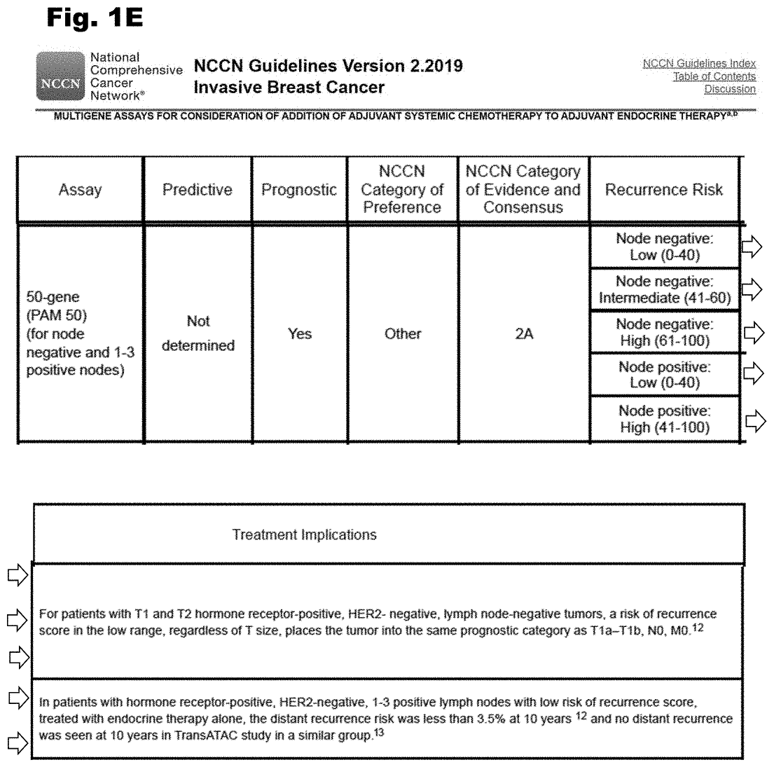

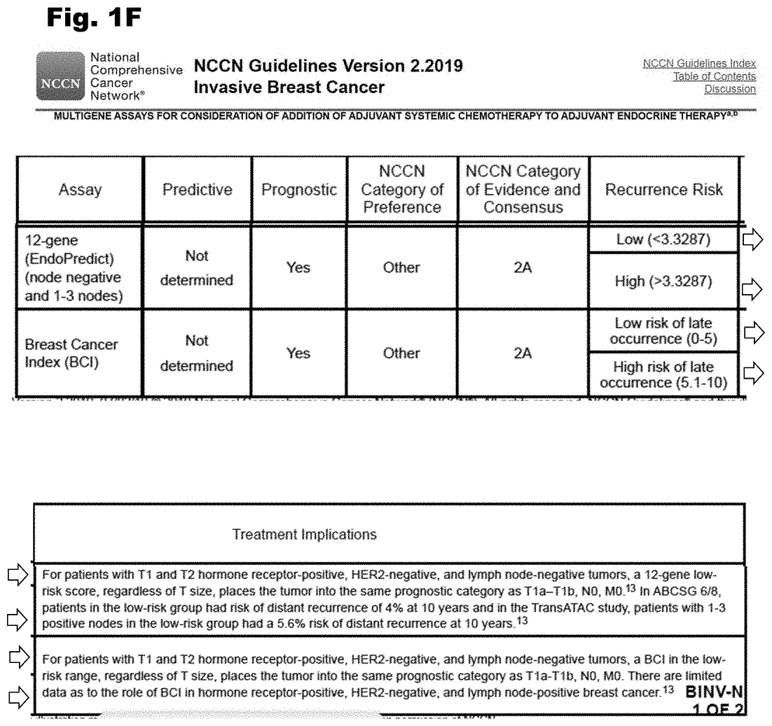

Several diagnostic tests are currently available in order to guide clinicians on the approach to monitoring and treating patients with breast cancer (FIGS. 1A to 1F). Most of these tests utilize molecular and genomic techniques in order to gain insight on the genetic aberrations within a neoplasm and potential associated risks, such as recurrence. In addition, the tests can inform personalized treatment options, for instance, the decision to utilize chemotherapy (including neoadjuvant or adjuvant chemotherapy), the strength, dose, and duration of a chemotherapeutic, to utilize endocrine therapy, and to utilize other treatment options (e.g., targeted therapy, immunotherapy). For a detailed discussion on the various diagnostic tests available for breast cancer, see O. M Fayanju, K. U. Park, and A. Lucci Ann. Surg. Oncol. 25, 512-19 (2018), the disclosure of which is herein incorporated by reference.

Diagnostic tests include the Oncotype Dx (Genomic Health, Redwood City, Calif.), Prosigna (NanoString Technologies, Seattle Wash.), MammaPrint (Agendia, Irvine, Calif.), EndoPredict (Myriad Genetics, Salt Lake City, Utah) and Breast Cancer Index (BCI) (Biotheranostics, Inc., San Diego, Calif.) (See FIGS. 1A to 1F).

Oncotype Dx is the most commonly used diagnostic test used for breast cancer in the United States. The test examines the expression of 21 genes, which is used to determine whether chemotherapy is indicated, especially in individuals with early-stage ER+, HER2−, lymph node negative (LN−) breast cancer. Oncotype Dx quantifies the likelihood of distant recurrence within 10 years, providing a score that indicates a high (≥31), intermediate (18-30), or low (0-17) likelihood of recurrence. It is noted that results indicating intermediate recurrence scores present a clinical conundrum for clinicians with respect to the indication of which treatment to perform.

Prosigna, which is based on the PAM50 classifier, is a diagnostic test that determines expression of 50 genes. The Prosigna test generates a risk of recurrence score (ROR) and assigns a tumor to one of four intrinsic subtypes: Luminal A, Luminal B, HER2+, and Basal-like. Based on ROR score and other clinical factors (including lymph node status), risk status is determined.

MammaPrint is a 70 gene expression assay profiled on a microarray to predict distant metastasis within 5 years in ER+/HER2− patients. MammaPrint can be utilized for patients with positive or negative lymph node status. Based on expression profile results, the molecular prognosis profile of low risk or high risk is determined.

EndoPredict is a 12-gene test to predict risk of distance recurrence 10 years post diagnosis in ER+/HER2− patients with a negative lymph node status or positive lymph node (1-3) status. Based on expression profile results, the molecular prognosis profile of low risk or high risk is determined.

Breast Cancer Index (BCI) combines proliferative and estrogen-signaling gene-expression signatures to predict distant recurrence 5 to 10 years post diagnosis in ER+ patients with a negative lymph node status or positive lymph node (1-3) status. BCI is intended to be utilized to determine whether a patient can benefit from extended (>5 year) adjuvant endocrine therapy.

Some individuals have an aggressive form of cancer, which may also include a persistent risk of recurrence and breast cancer death up to and beyond twenty years later. Often, from the current diagnostic tests available, it can be difficult to discern who is at risk of recurrence, especially late recurrence (e.g., >5 years). For instance, a subset of individuals with early stage ER+ breast cancer have a persistent risk of recurrence and death up to 20 years after diagnosis, but the current diagnostics have a difficult time identifying this subset. In fact, most current diagnostic assays fail to reliably predict beyond five years and, as time passes, clinical covariates continue to lose prediction power. Accordingly, there is a critical need to identify tumor characteristics that are more predictive of aggressiveness and risk of recurrence than the current available tests and standard clinical covariates (nodal status, tumor size and grade) in order to define subsets of patients with high-risk and low-risk cancers, including risk of recurrence. Having a better understanding of risk and relapse potential can help delineate which individuals would benefit from various therapies, such as extended endocrine therapy or higher dosage of a chemotherapeutic or molecularly targeted therapies.

Here, several embodiments are based on molecular tests that classify breast cancer into a reoccurrence risk subgroup (e.g., high, intermediate, low) and/or an integrative cluster (IntClust) (see C. Curtis, et al., (2012), cited supra). Classification into a risk subgroup can be performed by a number of statistical techniques, including (but not limited to) multi-state semi-markov Models, Cox Proportional Hazards models, shrinkage based methods, tree based methods, Bayesian methods, kernel based methods and neural networks.

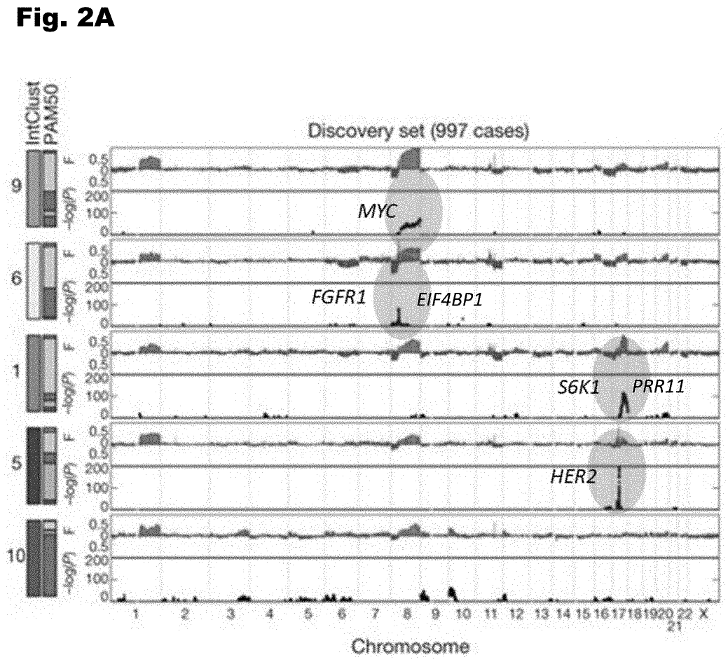

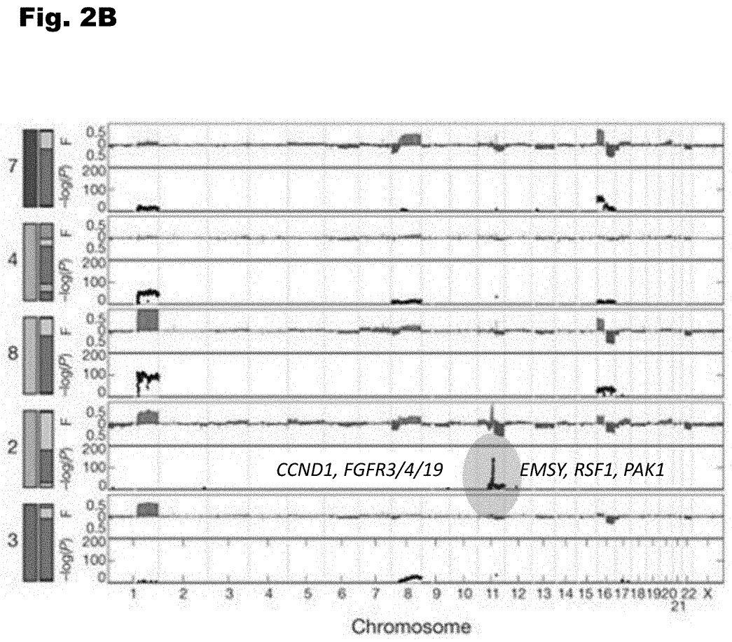

For clustering into an IntClust subgroup, a total of 11 IntClust subgroups are currently described, which were developed utilizing an unsupervised joint latent variable clustering of gene expression and copy number profiles that each breast cancer within the study harbored. A total of ˜1000 early stage breast cancers were used to develop the clusters, which were validated in another ˜1000 early stage breast cancers, and the results are shown in FIGS. 2A and 2B. CNA amplifications are depicted in red while CNA losses are depicted in blue. Note that 10 IntClust subgroups are depicted, each determined by the computational modeling, however, IntClust4 can be further divided into ER+ and ER− to yield 11 IntClust subgroups.

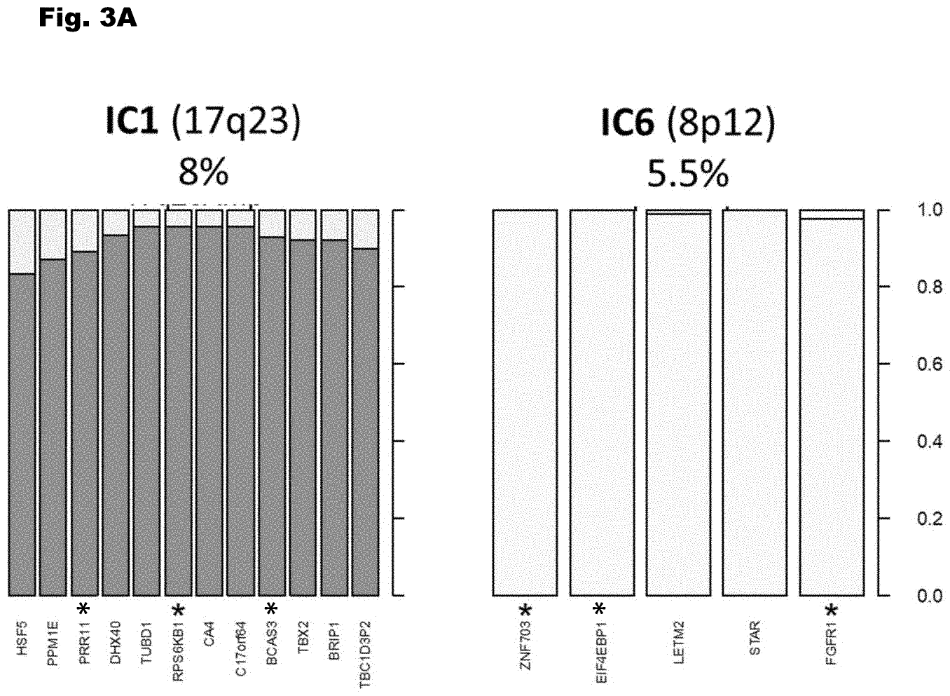

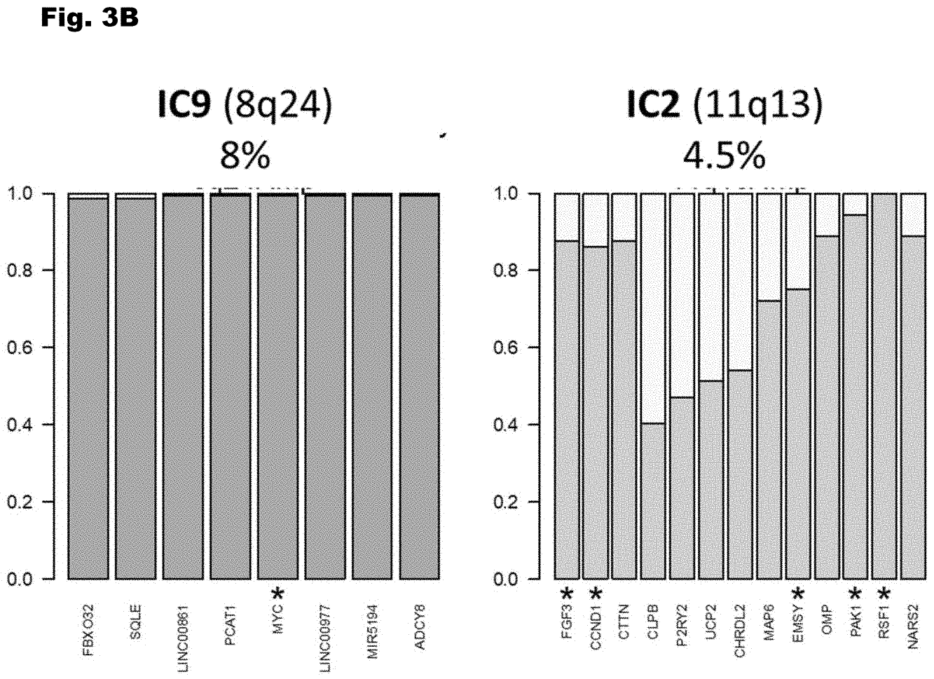

The IntClust subgroups are each characterized by the copy number aberrations (CNAs) and relative gene expression levels that are harbored within the cancer and are likely to be involved with the progression of cancer (i.e., molecular drivers of breast cancer). For example, IntClust subgroups 1, 2, 6, and 9 were found to account for approximately 25% of all ER+ tumors and each subgroup is enriched for characteristic copy number amplification events in various regions of the genome (see FIGS. 2A and 2B). Regarding IntClust1, it now known that the genes near 17q23 (e.g., RPS6KB1) are amplified and over-expressed. Likewise, IntClust2 has amplifications of genes CCND1, FGF3 (11q13.3) and 11q13.2 amplicon genes (e.g., EMSY, RSF1, PAK1), and these regions of the genome are frequently co-amplified with concomitant gene expression upregulation, suggesting oncogenic cooperation between these loci. Of note, the recurrent amplification of chromosome 8p12 and 11q13 suggests that these loci may cooperate to promote tumor development and progression. As such, they may need to be co-targeted in some patients. IntClust6 exhibits amplifications of the genes near 8p12 (e.g., FGFR1, ZNF703, EIF4EBP1). In addition, IntClust9 has amplification and over-expression of genes near 8q24 (e.g., MYC) and 20q13 (e.g., SRC3, NCOA3). In similar analysis, Intclust5 is characterized by amplification and over-expression in HER2/ERBB2, an oncogene that is well-understood to be a molecular driver of breast cancer. Shown in FIGS. 3A and 3B are the percentage of tumors in the cohort having CNA gain or amplification of genes that define IntClust subgroup that it has been assigned (note: FIGS. 3A and 3B include oncogenic drivers for each integrated cluster, which are asterisked, based on preclinical data).

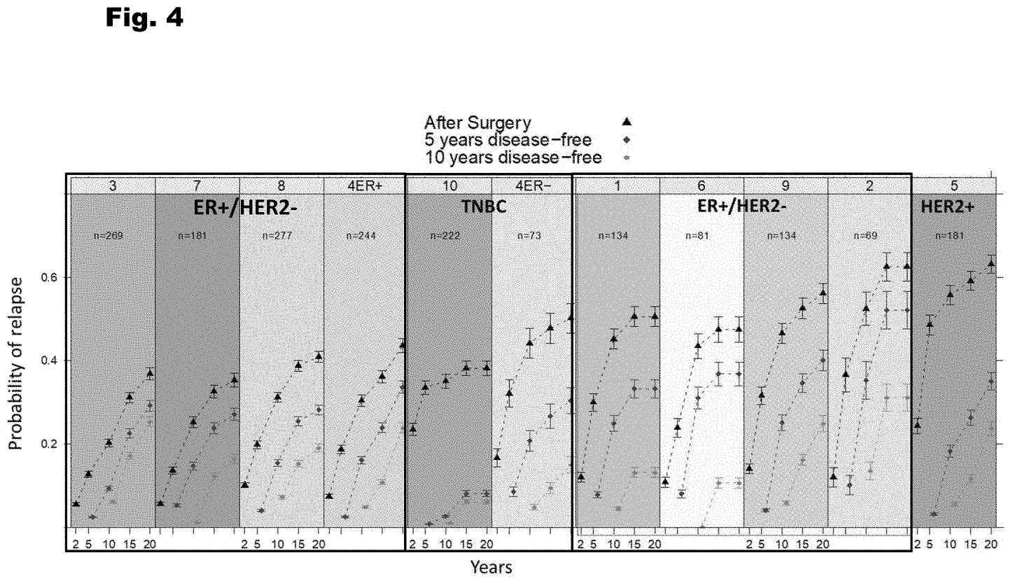

It is now known that particular IntClust subgroups confer aggressiveness and potential for relapse (FIG. 4). In other words, when a breast cancer is classified into a particular IntClust subgroup, the likelihood of the cancer to be aggressive and to relapse can be determined. This knowledge can also be used to determine courses of treatments and/or the necessity of continued monitoring. For example, subtyping into IntClust subgroups can inform whether to extend endocrine therapy in high-risk populations, avoid endocrine therapy in patents that are intrinsically endocrine resistant, applying targeted therapy based on molecular drivers of the IntClust subgroup, and the appropriate choice and treatment regimen of chemotherapeutics.

The use of these integrated clusters was found to improve prediction of late distant relapse (especially relapse after 5 years) better than standard clinical covariates and current diagnostic methods, which is corroborated in an external validation cohort. It was also found that a subgroup of triple-negative breast cancer patients rarely recur after 5 years while others remain at risk. After distant recurrence, tumor subtype continues to dictate the rate of subsequent metastases, underscoring the importance of classifying tumors accordingly. Based on these findings, several embodiments are directed to identifying individuals having a particular risk of aggressive cancer and relapse, as determined by a diagnostic method. Various embodiments treat and/or monitor an individual based on their cancer's aggressiveness and risk of relapse.

FIG. 4 shows the results of a study to investigate aggressiveness and relapse of breast cancers within each classification. Here a non-homogenous (semi) Markov chain model was utilized to delineate the spatio-temporal dynamics of breast cancer relapse across the IntClust subgroups (see Exemplary Embodiments). The results from this model illustrate that various subgroups have a much higher likelihood of relapse, especially beyond the 5 or even 10 or 15 year marks.

Shown in FIG. 4 is each of the 11 IntClust subgroups and the probability of relapse from three timepoints: surgery, 5 years after surgery and disease free, and 10 years after surgery and disease free. The results are ordered by the risk of relapse, with the subgroups having the least risk of relapse on the left on the most risk of relapse on the right. Based on these results, groups can be split into high risk groups and lower risk groups. Lower risk groups include IntClust3, IntClust7, IntClust8, IntClust4ER+, and IntClust10. High risk groups include IntClust4ER−, IntClust1, IntClust6, IntClust9, IntClust2, and IntClust5.

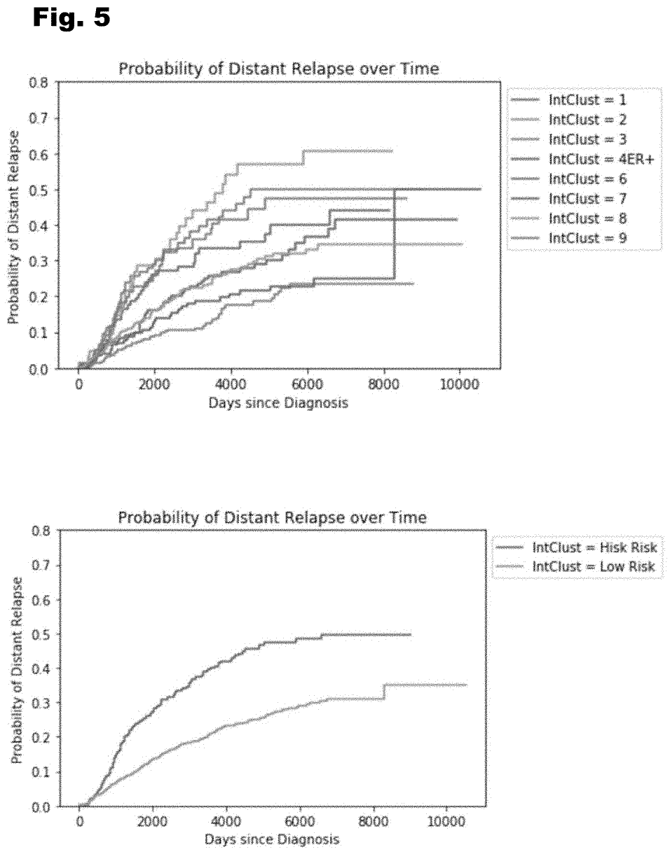

Provided in FIG. 5 are cumulative incidence plots (i.e., 1—Kaplan Meier estimates) displaying the risk of distant relapse among ER+/HER2− breast cancer patients over time, based on clinical outcome data. As can be seen in the top panel of FIG. 5, IntClust subgroups 2, 9, 6 and 1 have an increased probability of distant relapse. The lower panel of FIG. 5 compares high risk subgroups (IntClust subgroups 1, 2, 6 and 9) compared to lower risk subgroups (IntClust subgroups 3, 4ER+, 7, and 8). The results show a clear distinction of risk between the two subgroups.

IntClust10 and IntClust4ER− have a clinical classification of being triple negative breast cancer (TNBC), which means they are ER−, HER2−, and PR−. TNBC occurs in 10% to 20% of breast cancers and is more likely to affect younger people. TNBC can be difficult to treat, due to its aggressiveness and potential for recurrence. However, the results of the IntClust study show that those in IntClust10 have a very low likelihood of recurrence after 5 years disease free. On the contrary, IntClust4ER− has a relatively high likelihood of recurrence, even after 5 years or even after 10 years of being disease free. Accordingly, in a number of embodiments, an individual having TNBC is assessed to determine which IntClust subgroup the cancer is classified into, and thus performing a treatment based on the result.

IntClust3, IntClust7, IntClust8, and IntClust4ER+ are all ER+/HER2− and have a modest risk of recurrence. IntClust1, IntClust6, IntClust9, and IntClust2, on the other hand, are ER+/HER2− and have a high and persistent risk of recurrence. Accordingly, in various embodiments, when a cancer is classified as a high risk ER+/HER2−, a more aggressive treatment regimen may be beneficial (e.g., adjuvant chemotherapy in addition to endocrine therapy). In addition, the oncogenic genomic drivers of the high risk of recurrence groups can targeted directly by specific targeted treatments. For instance, in some embodiments, IntClust1 cancers are treated with mTOR pathway antagonists (e.g., everolimus, temsirolimus, sirolimus, rapamycin), AKT1 antagonists (e.g., ipatasertib, capivasertib (AZD5363)), AKT1/RPS6KB1 antagonists (e.g., M2698), RPS6KB1 antagonists (e.g., LY2584702), PI3K antagonists (e.g., alpelisib, buparlisib (BKM120), pictilisib (GDC-0941)), elF4A antagonists (e.g., zotatifin), elF4E antagonists (e.g., rapamycin, rapamycin analogues, ribavirin, AZD8055),or a combination thereof. In various embodiments, IntClust2 cancers are treated with epigenetically targeted therapies, CDK4/6 antagonists (e.g., palbociclib, ribociclib, abemaciclib), FGFR pathway antagonists (e.g., lucitanib, dovitinib, AZD4547, erdafitinib, infigratinib (BGJ398), BAY-1163877, ponatinib), PARP antagonist (e.g., niraparib, olaparib), homologous recombination deficiency (HRD)-targeted therapies, PAK1 antagonist (e.g., IPA3), elF4A antagonists (e.g., zotatifin), elF4E antagonists (e.g., rapamycin, rapamycin analogues, ribavirin, AZD8055), or a combination thereof. In some embodiments, IntClust6 cancers are treated with FGFR pathway antagonists (e.g., lucitanib, dovitinib, AZD4547, erdafitinib, Infigratinib (BGJ398), BAY-1163877, Ponatinib), elF4A antagonists (e.g., zotatifin), elF4E antagonists (e.g., rapamycin, rapamycin analogues, ribavirin, AZD8055), or a combination thereof. And in various embodiments, IntClust9 cancers are treated with selective estrogen receptor degraders (SERDs) (e.g., fulvestrant, GDC-9545, SAR439859 (SERD '859), RG6171, AZD9833), the proteolysis targeting chimera (PROTAC) ARV-471, SRC3 antagonists (e.g., SI-2), MYC antagonists (e.g., omomyc), BET bromodomain antagonists (e.g., JQ1, PROTAC ARV-771), elF4A antagonists (e.g., zotatifin), elF4E antagonists (e.g., rapamycin, rapamycin analogues, ribavirin, AZD8055), or a combination thereof.

### Methods to Classify and Stratify Breast Cancers

Several embodiments are directed to classifying and/or stratifying risk of a breast cancer for diagnostic purposes. In some embodiments, a breast cancer is classified into a particular IntClust subgroup. In some embodiments, a breast cancer is stratified by risk potential (e.g., low, intermediate or high risk).

In a number of embodiments, a breast cancer is classified into an integrated cluster (IntClust), as those described in C. Curtis, et al. (2012), cited supra. Each of the eleven IntClust subgroups have a relatively defined set of CNAs as determined by clustering analysis (FIG. 2). It is noted that IntClust 4 can be further divided into ER+ and ER− to round out the eleven subgroups. By using the IntClust classification, in various embodiments, a breast cancer is classified into one of the eleven subgroups. Although IntClust classification is described, other genomic driver classification methods of breast cancer can be used in accordance with some embodiments.

It is now understood that ER+/HER2− breast cancers that fall within various IntClust subgroups are highly aggressive with high risk of relapse, including subgroups 1, 2, 6, and 9. Likewise, cancers that fall within IntClust subgroups 3, 7, 8, and 4ER+ are less aggressive and have lower risk of relapse. Accordingly, various embodiments classify a breast cancer into an IntClust subgroup to determine the aggressiveness and risk relapse of the cancer. In a similar manner, TNBC can be classified into high risk subgroup IntClust4ER− or lower risk subgroup IntClust10.

To classify an individual into an IntClust, gene expression and/or CNA data is obtained from a breast cancer. CNAs can be detected by a number of methods. In some embodiments, DNA of a cancer is extracted from an individual and processed to detect CNA levels. In various embodiments, RNA of a cancer is extracted and processed to detect expression levels of a number of genes, which can be utilized to determine aberrations in copy number. It should be further understood that various embodiments can utilize both DNA and RNA extractions to determine molecular subtypes. Additionally, since DNA methylation is highly correlated with gene expression as is chromatin accessibility (or state), DNA methylation or chromatin accessibility profiling (ATAC-seq) is used in a number of embodiments to determine Integrative Cluster membership or Integrative Subtype.

In a number of embodiments, features used to determine a breast cancer's integrative subtype include CNA and/or expression data. Accordingly, a computational classifier can utilize copy number features and/or gene expression features but may also use DNA (gene/CpG) methylation features and/or accessible DNA peaks derived from DNA methylation or chromatin (DNA) accessibility analysis of a breast cancer. In some embodiments, copy number features are matched by either genomic position or gene name. In various embodiments, expression features or matched to a probe that detects expression. After features are matched, various embodiments scale each feature to a z-score and may include other normalization methods. In numerous embodiments, the matched features are entered into the computational classifier such that the classifier determines which subgroup the breast cancer falls within. In some embodiments, the previously described unsupervised joint latent variable clustering approach is used (described in the publication of C. Curtis, et al., (2012) or the integrative subtype (iC10) classifier as described in the publication of H. R. Ali, et al., (2014), which can be found as a CRAN R package labeled iC10 (https://cran.r-project.org/web/packages/iC10/index.html), cited supra.

In a various embodiments, molecular class prediction models include (but not limited to) shrinkage based methods, logistic regression, support vector machines with a linear kernel, support vector machines with a gaussian kernel, and neural networks, each of which can independently be used to classify a breast cancer into the 11 integrative subtypes. Class prediction models can be based on various molecular features including copy number features and/or gene expression features, DNA (gene/CpG) methylation features and/or accessible DNA peaks derived from chromatin accessibility analysis of a breast cancer. In some embodiments, a top scoring pairs (TSP) classification approach (or variations thereof) is used, in which a pair of variables whose relative ordering can be used for accurately predicting the class label of a sample. An example of this approach is implemented in the Rgtsp package (V. Popovici, E. Budinska, and M. Delorenzi, Bioinformatics 27, 1729-30 (2011), the disclosure of which is herein incorporated by reference). Further, in some embodiments, molecular class prediction is extended to perform absolute subtype assignments, such as utilizing the AIMS algorithm described by Paquet et al. (E. R. Paquet and M. T. Hallet, J. Natl. Cancer Inst. 107, 357 (2014), the disclosure of which is herein incorporated by reference).

Nucleic acids or protein can be extracted or examined within a tissue biopsy of the tumor and/or from an individual's bodily fluids (e.g., blood, plasma, urine) by a number of methodologies, as understood by practitioners in the field. Once extracted, nucleic acids can be processed and prepared for detection. Methods of detection include (but are not limited to) hybridization techniques (e.g., in situ hybridization (ISH), nucleic acid proliferation techniques, and sequencing. Various molecular techniques can be used, including (but not limited to) microarray based gene expression, microarray/SNP array based copy number inference, RNA-sequencing, targeted (capture) RNA-sequencing, exome sequencing, whole genome sequencing (WES/WGS), targeted (panel) sequencing, NanoString nCounter for gene expression, NanoString nCounter for copy number inference, Nanostring Digital Spatial Profiling (for in situ protein expression/RNA expression), DNA-ISH, RNA-ISH, RNAScope, DNA Methylation assays, and ATAC-seq, and immunohistochemistry (IHC).

In several embodiments, CNA and/or expression levels are defined relative to a known result. In some instances, CNA and/or expression levels of a test sample is determined relative to a control sample or molecular signature (i.e., a sample/signature with a known classification). A control sample/signature can either be healthy tissue (i.e., null control), a known positive control, or any other control that is desired. Accordingly, when the CNA and/or expression levels of a test sample is compared to one or more controls, the relative CNA and/or expression levels can determine which genomic driver subgroup the test sample falls within. In some instances, gene expression levels are determined relative to a stably expressed biomarker (i.e., endogenous control). In some instances, when gene expression levels exceed a certain threshold relative to a stably expressed biomarker, the level of expression is indicative of a particular genomic driver subgroup. In some instances, CNA and/or expression levels are determined absolutely. In some instances, various CNA and/or expression level thresholds and ranges can be set to classify genomic driver subgroups and thus used to indicate which subgroup a test sample falls within. It should be understood that methods to define CNA and/or expression levels can be combined, as necessary for the applicable assessment. Utilizing transcript expression levels, CNA levels, DNA methylation levels, chromatin (DNA) accessibility peaks, or any combination thereof, a breast cancer can be classified.

Genomic loci and/or genes are detected in accordance with various embodiments. In some embodiments, detection of a particular set of genomic CNAs and/or transcript expression classifies a breast cancer into a particular IntClust subgroup. Referring back to FIGS. 3A and 3B, CNAs in various loci are demonstrative of a number of IntClust subgroups. For example, IntClust subgroups 1, 2, 6, and 9 were found to account for approximately 25% of all ER+ tumors and each is enriched for a characteristic copy number amplification events of various sections of the genome. Regarding IntClust1, it now known that the genes near 17q23 including (but not limited to) RPS6KB1, HASF5, PPM1E, PRR11, DHX40, TUBD1, CA4, C17orf64, BCAS3, TBX2, BRIP1, and TBC1D3P2 are amplified. Likewise, IntClust2 has amplifications of genes CCND1, FGF3 (at 11q13.3) and 11q13.2 amplicon genes including (but not limited to) EMSY, RSF1, PAK1, CTTN, CLPB, P2RY2, UCP2, CHRDL2, MAP6, OMP, and ARS2. IntClust6 exhibits amplifications of the genes near 8p12 including (but not limited to) FGFR1, ZNF703, EIF4EBP1, LETM2, and STAR. In addition, IntClust9 has amplification of genes near 8q24 including but limited to MYC, FBXO32, LINC00861, PCAT1, LINC00977, MIR5192, and ADCY8 and near 20q13 including (but not limited to) SRC3, NCOA3. Accordingly, detection of an amplification (CNA or expression) of a locus or gene, or a combination of loci and/or genes, can be utilized to indicate a particular IntClust classification.

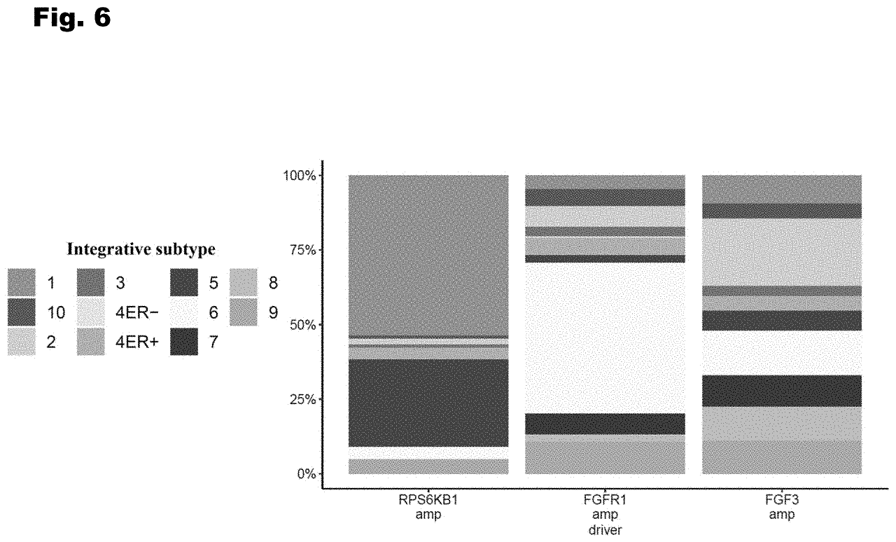

In a number of embodiments, classification of breast cancer is performed utilizing a computational model based on multiple genomic copy number aberrations, multiple gene expression profiles, DNA methylation levels, chromatin (DNA) accessibility peaks, or any combination thereof, which may provide a more accurate classification than copy number state/gene expression at a single chromosomal locus. For instance, amplifications of the genes RPS6KB1, FGFR1, and FGF3 occur within a variety breast cancer IntClust subgroups, including those that have low aggressiveness and risk of relapse. As can be seen in FIG. 6, approximately 50% of breast cancers having an RPS6KB1 gain or amplification are classified into IntClust1, however RPS6KB1 copy number alteration is also detected within several more IntClust subgroups. Likewise, approximately 50% of breast cancers having an FGFR1 amplification are classified into IntClust6 and the amplification can be detected within all the other subgroups. FGF3 amplification is fairly evenly distributed between the IntClust subgroups. Thus, it may be beneficial to utilize a trained computational model such that a breast cancer can be more accurately classified into the appropriate subtype (e.g., IntClust classifier).

A number of embodiments utilize statistical computation to stratify breast cancer recurrence risk (e.g., high, intermediate, low). In various embodiments, statistical computation models include (but not limited to) multi-state semi-markov Models, Cox Proportional Hazards models, shrinkage based methods, tree based methods, Bayesian methods, kernel based methods and neural networks. In some embodiments, thresholds are utilized to separate higher risk scores from lower risk scores. In several embodiments, features used to train statistical models and/or to predict risk of recurrence in breast cancer include (but not limited to) clinical data, age, cancer stage, number of tumor positive lymph nodes, size of tumor, grade of tumor, surgery performed, treatment performed, basic molecular identities, and integrative subtype classification/membership. Age of the patient can be coded as a continuous value (and potentially trimmed to avoid excessively high values (e.g., age>80). Clinical stage (values ranging from 1-4), can be included as a continuous value or as a factor or can be grouped as high (3-4) vs low (1-2) stage. Positive lymph nodes can be included as a continuous value (potentially trimmed to avoid excessively high values). The number of positive lymph nodes this can also be categorized as lymph node negative versus positive or amongst positive, graded as low (1 positive node), medium (2-3 positive nodes), high (4-9 positive nodes), very high (>=10 positive nodes) or variations thereof. Size of tumor can be used as a continuous value, which can be trimmed to avoid excessive high values). Size of tumor can also be categorized (e.g., staging system: T1<20mm, T2 (20-50), T3 (>50)). The grade of tumor can be used as a continuous value or as a category (1-3) or high (3) vs low (1,2). In some embodiments, classifiers include the CTS5 algorithm, which is based encoding of lymph node, size, grade may be incorporated as follows:

0.438×nodes+0.988×(0.093×size −0.001×size2+0.375×grade+0.017×age)

(for more on CTS5 algorithm, see M. Dowsett, et al., J. Clin. Oncol. 36, 1941-48 (2018), the disclosure of which is herein incorporated by reference). Basic molecular identities include status of estrogen receptor (ESR1), Progesterone receptor (PGR), human epidermal growth factor receptor 2 (HER2/ERBB2) and MKI67 based on clinical pathology reports and/or inferred from gene expression data. Surgery types can include breast conserving or mastectomy. Treatment type can include hormonal, chemotherapy, targeted therapy, where agents may be specified or grouped more broadly and treatment duration included. Various embodiments also utilize germline genetic variants, ethnicity, general health data, and/or treatment regimes. In some embodiments, the Predict Tool (https://breast.predict.nhs.uk) or components thereof can be utilized in the model.

In some embodiments, features can be derived from integrated subtype clusters (e.g., IntClust classification) and included in the model. These features can be integrative subtype membership or the posterior probability of membership to a given cluster. An integrative subtype is coded individually as a logical feature. Distance to the centroid of each subgroup can be utilized. Any score derived from the IC classifier can also be utilized. In some embodiments, risk of relapse prediction on specific subpopulations is utilized, such as ER+/HER2-patients or triple negative breast cancer patients. Amongst ER+/HER2− patients, high risk (IntClust1, IntClust2, Intclust6 or IntClust10) versus lower risk (IntClust3, IntClust4, Intclust7 or IntClust8) categories may be considered. Likewise, TNBC classified into IntClust4ER− are determined to be aggressive and have high risk, whereas TNBC classified into IntClust10 are determined to have lower risk.

In a number of embodiments, a multi-state Cox reset model is utilized, which is a statistical model that accounts for different disease states (loco-regional recurrence and distal recurrence), different timescales (time from diagnosis and time from relapse), competing causes of death (cancer death or other causes), clinical covariates or age effects, and distinct baseline hazards for different molecular subgroups (see H. Putter, M. Fiocco, & R. B. Geskus, Stat. Med. 26, 2389-430 (2007); O. Aalen, O. Borgan, & H. Gjessing, Survival and Event History Analysis —A Process Point of View. (Springer-Verlag New York, 2008); and T. M. Therneau & P. M. Grambsh, Modeling Survival Data: Extending the Cox Model. (Springer-Verlag New York, 2000); the disclosures of which are each herein incorporated by reference). In many embodiments, a multistate statistical model is fit to the dataset, such that the chronology of breast cancer, starting with surgical excision of the primary tumor, followed by the development of loco-regional and/or distant recurrence and accounting by competing risks of death due to cancer or other causes are accounted. In some embodiments, the hazards of occurrence of each of these states are modeled with a non-homogenous semi-Markov Chain with two absorbent states (Death/Cancer and Death/Other). For more on multi-state Cox models, see the description in the Exemplary Embodiments.

Cox proportion hazard models are statistical survival models that relate the time that passes to an event and the covariates associated with that quantity in time (See D. R. Cox, J. R. Stat. Soc. B 34, 187-220 (1972), the disclosure of which is herein incorporated by reference). To utilize Cox proportional hazards models, in some embodiments, clinical, molecular, and integrative subtype features are included. In some embodiments, features can be linear and/or polynomial transformed and interaction can include variable selection. In some embodiments, to further simplify the model, stepwise variable selection can be incorporated into the cross validation scheme. Any appropriate computational package can be utilized and/or adapted, such as (for example), the RMS package (https://www.rdocumentation.org/packages/rms).

Shrinkage based methods include (but not limited to) regularized lasso (R. Tibshirani Stat. Med. 16, 385-95 (1997), the disclosure of which is herein incorporated by reference), lassoed principal components (D. M. Witten and R. Tibshirani Ann. Appl. Stat. 2, 986-1012 (2008), the disclosure of which is herein incorporated by reference), and shrunken centroids (R. Tibshirani, et al., Proc. Natl. Acad. Sci. USA 99, 6567-72 (2002), the disclosure of which is herein incorporated by reference). Any appropriate computation package can be utilized and/or adapted, such as (for example), the PAMR package for shrunken centroid (https://www.rdocumentation.org/packages/pamr/versions/1.56.1).

Tree based models include (but not limited to) survival random forest (H. Ishwaran, et al., Ann. Appl. Stat. 2, 841-60 (2008), the disclosure of which is herein incorporated by reference) and random rotation survival forest (L. Zhou, H. Wang, and Q. Xu, Springerplus 5, 1425 (2016), the disclosure of which is herein incorporated by reference). In some embodiments, the hyperparameter corresponds to the number of features selected for each tree. Any appropriate setting for the number of trees can be utilized, such as (for example) 1000 trees. Any appropriate computation package can be utilized and/or adapted, such as (for example), the RRotSF package for random rotation survival forest (https://github.com/whcsu/RRotSF).

Bayesian methods include (but not limited to) Bayesian survival regression (J. G. Ibrahim, M. H. Chen, and D. Sinha, BAYESIAN SURVIVAL ANALYSIS, Springer (2001), the disclosure of which is herein incorporated by reference) and Bayes mixture survival models (A. Kottas J. Stat. Pan. Inference 3, 578-96 (2006), the disclosure of which is herein incorporated by reference). In some embodiments, sampling is performed with a multivariate normal distribution or a linear combination of monotone splines (See B. Cai, X. Lin, and L. Wang, Comput. Stat. Data Anal. 55, 2644-51 (2011), the disclosure of which is herein incorporated by reference). Any appropriate computation package can be utilized and/or adapted, such as (for example), the ICBayes package (https://www.rdocumentation.org/packages/ICBayes/versions/1.0/topics/ICBayes).

Kernel based methods include (but not limited to) survival support vector machines (L. Evers and C. M. Messow, Bioinformatics 24, 1632-38 (2008), the disclosure of which is herein incorporated by reference), kernel Cox regression (H. Li and Y. Luan, Pac. Symp. Biuocomp. 65-76 (2003), the disclosure of which is herein incorporated by reference), and multiple kernel learning (O. Dereli, C. Oguz, and M. Gonen Bioinformatics (2019), the disclosure of which is herein incorporated by reference). It is to be understood that kernel based methods can include support vector machines (SVM) and survival support vector machines with polynomial and Gaussian kernel, where hyperparameter C specifies regularization (See L. Evers and C. M. Messow, cited supra). In some embodiments, multiple kernel learning (MKL) approaches combine features in kernels, including kernels embed clinical information, molecular information and integrative subtype. Any appropriate computation package can be utilized and/or adapted, such as (for example), the path2surv package (https://github.com/mehmetgonen/path2surv).

Neural network methods include (but not limited to) DeepSury (J. L. Katzman, et al., BMC Med. Res. Methodol. 18, 24 (2018), the disclosure of which is herein incorporated by reference), and SuvivalNet (S. Yousefi, et al., Sci. Rep. 7, 11707 (2017), the disclosure of which is herein incorporated by reference). Any appropriate computation package can be utilized and/or adapted, such as (for example), the Optunity package (pypi.org/project/Optunity/).

In several embodiments, in order to ensure that a model is not overfitted, models are trained using an X-times, and cross validated X-fold scheme (e.g., 10-fold training, 10-fold cross validation). Sample data can be split into subsets, and some data is used to train the model and some data is used to evaluate the model. By using this method, it can be assured that all data are validated at least once and no sample is used for both training and validation at the same time, all while the X-fold cross validation minimized sampling bias. A training/cross-validation approach also enables evaluation of the stability of the predictions by calculating confidence intervals, which facilitates model comparisons. Additionally, an internal cross validation scheme can be employed for hyperparameter specification.

While specific examples of processes for molecularly classifying and stratifying risk of a breast cancer are described above, one of ordinary skill in the art can appreciate that various steps of the process can be performed in different orders and that certain steps may be optional according to some embodiments. As such, it should be clear that the various steps of the process could be used as appropriate to the requirements of specific applications. Furthermore, any of a variety of processes for molecular classification and risk stratification appropriate to the requirements of a given application can be utilized in accordance with various embodiments.

Numerous embodiments are directed towards combining risk prediction models that incorporate integrative subtype information with other multigene signatures, including (but not limited to) Oncotype Dx (Genomic Health, Redwood City, Calif.), Prosigna (NanoString Technologies, Seattle Wash.), MammaPrint (Agendia, Irvine, Calif.), EndoPredict (Myriad Genetics, Salt Lake City, Utah), Breast Cancer Index (BCI) (Biotheranostics, Inc., San Diego, Calif.). Of particular interest is the combination of Oncotype DX with the Integrative Subtype (IntClust). As stated previously, Oncotype DX yields a result indicating one of: high, intermediate or low likelihood of recurrence and the treatment choice for an intermediate likelihood can be a conundrum for clinicians. However, when Oncotype DX is combined with an integrative clustering technique, breast cancers that would normally fall within the intermediate risk group can be better stratified resulting in clear results of high and lower risk. Details of combining Oncotype DX with an integrative clustering technique is described within the exemplary embodiments section. Combinations with Prosigna, MammaPrint, BCI, and EndoPredict have also shown improvements in diagnostic stratification, as detailed in the Exemplary Embodiments.

### Methods of Detecting Copy Number Aberrations & Gene Expression

Aberrations in copy number can be detected by a number of methods in accordance with various embodiments, as would be understood by those skilled in the art. In several embodiments, CNAs are detected directly from genomic DNA and/or inferred from RNA transcript expression. Accordingly, in some embodiments CNA analysis is used to classify breast cancers. In some embodiments, RNA expression analysis is used to classify breast cancer. And in some embodiments, analysis of both CNA and RNA expression is used to classify a breast cancer.

The source of nucleic acids (e.g., DNA and RNA) to determine expression can be derived de novo (i.e., from a biological source). Several methods are well known to extract nucleic acids from biological sources. Generally, nucleic acids are extracted from cells or tissue, then prepped for further analysis. Alternatively, DNA and/or RNA can be observed within cells, which are typically fixed and prepped for further analysis. The decision to extract nucleic acids or fix tissue (via formalin fixation and paraffin embedding (FFPE)) for direct examination depends on the assay to be performed, as would be understood by those skilled in the art. In some embodiments, DNA and/or RNA is extracted from tissue that is fixed.

In several embodiments, nucleic acids are extracted and/or examined in the type of cells and tissues to be treated. In many cases, the cells to be treated are neoplastic cells of a breast cancer of an individual, which can be extracted in a biopsy. In some embodiments, nucleic acids are extracted from blood or serum, which can include circulating tumor DNA, for analysis. The precise source to extract and/or examine nucleic acids can depend on the assay to be performed, the availability of a biopsy, and preference of the practitioner.

A number of assays are known to measure and quantify genomic loci copy numbers and transcript expression. CNAs and RNA expression levels can be determined by a number of methods, including (but are not limited to) hybridization techniques (e.g., in situ hybridization (ISH), nucleic acid proliferation techniques, and sequencing. Various molecular techniques can be used, including (but not limited to) microarray based gene expression, microarray/SNP array based copy number inference, RNA-sequencing, targeted (capture) RNA-sequencing, exome sequencing, whole genome sequencing (WES/WGS), targeted (panel) DNA sequencing (including Memorial Sloan Kettering Cancer Center Integrated Mutation Profiling of Actionable Cancer Targets (MSK-IMPACT), Foundation Medicine CDx, Stanford Tumor Actionable Mutation Panel (STAMP) (see moleculargenetics.stanford.edu/solid_tumors.htm), nanoString nCounter for gene expression, nanoString nCounter for copy number inference, nanoString Digital Spatial Profiler forcludes protein and RNA expression, DNA-ISH, RNA-ISH, RNAScope, DNA Methylation assays, and ATAC-seq.

Several embodiments are directed towards classifying integrative subtype from targeted sequencing data derived from a gene panel, such as those built by academic centers (e.g., UCSF500 Cancer Gene Panel (San Francisco, CA)) or companion diagnostic assays intended for other uses such as Foundation One CDx (Foundation Medicine, Cambridge, MA), and MSK-IMPACT (Memorial Sloan Kettering Cancer Center, New York, NY) or Stanford Tumor Actionable Mutation Panel (STAMP) (Stanford, Stanford, CA). Provided sufficient gene coverage is included within the panel, the various embodiments of algorithms described herein can be utilized. In some embodiments, a gene panel designed for breast cancer assessment is utilized. In some embodiments, a gene panel designed for chromatin regulatory gene assessment is utilized.

Several embodiments are directed to targeted detection of CNAs or gene transcripts. Accordingly, in many embodiments probes and/or primers are utilized to detect specific genes and/or genomic loci that are indicative of IntClust subgroups either directly or via a computational model as described herein.

As understood in the art, only a portion of a genomic locus or gene may need to be detected in order to have a positive detection. In some instances, genes can be detected with identification of as few as ten nucleotides. In many hybridization techniques, detection probes are typically between ten and fifty bases, however, the precise length will depend on assay conditions and preferences of the assay developer. In many amplification techniques, amplicons are often between fifty and one-thousand bases, which will also depend on assay conditions and preferences of the assay developer. In many sequencing techniques, genomic loci and transcripts are identified with sequence reads between ten and several hundred bases, which again will depend on assay conditions and preferences of the assay developer.

It should be understood that minor variations in gene sequence and/or assay tools (e.g., hybridization probes, amplification primers) may exist but would be expected to provide similar results in a detection assay. These minor variations are to include (but not limited to) insertions, deletions, single nucleotide polymorphisms, and other variations due to assay design. In some embodiments, detection assays are able to detect genomic loci and transcripts having high homology but not perfect homology (e.g., 70%, 80%, 90%, or 95% homology). As understood in the art, the longer the nucleic acid polymers used for hybridization, less homology is needed for the hybridization to occur.

It should also be understood that several gene transcripts have a number isoforms that are expressed. As understood in the art, many alternative isoforms would be understood to confer similar indication of molecular classification, and thus cancer aggressiveness and risk of relapse. Accordingly, alternative isoforms of gene transcripts are also covered in some embodiments.

In many embodiments, an assay is used to measure and quantify CNAs and transcript expression. The results of the assay can be used to determine relative CNA and transcript expression of a tissue of interest. For example, the nanoString nCounter, which can quantify up to several hundred nucleic acid molecule sequences in one microtube utilizing a set of complement nucleic acids and probes, which can be used to determine CNA and transcript expression of a set of genomic loci and/or gene transcripts. The resulting copy number and expression can be used to classify the sample either directly or utilizing a computational model as described herein, thus determining the cancer's aggressiveness and risk of relapse. Based on the cancer's aggressiveness and risk of relapse, the cancer can be treated accordingly.

### Kits for Detection Copy Number Aberrations and Gene Expression

In several embodiments, kits are utilized for evaluating individuals for breast cancer risk, wherein the kits can be used to detect genetic aberrations in biomarkers and/or prepare for a sequencing reaction as described herein. For example, the kits can be used to detect any one or more of the gene biomarkers described herein, which can be used to determine aggressiveness and metastatic potential. The kit may include one or more agents for determining genetic aberrations and/or preparing sequencing, a container for holding a biological sample (e.g., tumor or liquid biopsy) obtained from a subject; and printed instructions for reacting agents with the biological sample to detect the presence or amount of one or more genetic aberrations within biomarker genes derived from the sample. The agents may be packaged in separate containers. The kit may further comprise one or more control reference samples and reagents for performing a biochemical assay, enzymatic assay, immunoassay, hybridization assay, or sequencing assay.

A kit can include one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing written instructions for methods of detecting aberrations from tumor and/or liquid biopsies.

In several embodiments, kits are used to measure and quantify CNAs and transcript expression. A nucleic acid detection kit, in accordance with various embodiments, includes a set of hybridization-capable complement sequences and/or amplification primers specific for a set of genomic loci and/or expressed transcripts. In some instances, a kit will include further reagents sufficient to facilitate detection and/or quantitation of a set of genomic loci and/or expressed transcripts. In some instances, a nucleic acid detection kit will be able to detect and/or quantify for at least 5, 10, 15, 20, 25, 30, 40 or 50 loci and/or genes. In some instances, a nucleic acid detection kit will include an array to detect and/or quantify for at least 100, 200, 300, 400, 500 or 1000 loci and/or genes. In some instances, a kit will be able to detect and/or quantify thousands or more genes via an array or sequencing technique.

In a number of embodiments, a set of hybridization-capable complement sequences are immobilized on an array, such as those designed by Affymetrix or Illumina. In many embodiments, a set of hybridization-capable complement sequences are linked to a “barcode” to promote detection of hybridized species and provided such that hybridization can be performed in solution, such as those designed by nanoString. In several embodiments, a set of primers (and, in some cases probes) to promote amplification and detection of amplified species are provided such that a PCR can be performed in solution, such as those designed by Applied Biosystems of ThermoScientific (Foster City, Calif).

Many embodiments are directed to a kit being utilized as a companion diagnostic. Accordingly, in various embodiments, a kit is utilized to classify a breast cancer, which is then used to determine a particular treatment. For instance, a kit can be utilized to determine aggressiveness and risk of relapse of a breast cancer to determine the appropriate treatment. In some embodiments, a kit determines whether a breast cancer is a high risk, intermediate risk, or low risk, which then infers a more aggressive or less aggressive treatment, respectively. In some embodiments, a kit determines the molecular pathology of the breast cancer, which then infers whether to use a treatment that directly targets one or more oncogenic drivers.

### Treatment of Breast Cancer Determined by Molecular Characterization

A number of embodiments are directed to classifying and treating breast cancer. In several embodiments, a breast cancer is molecularly classified based and/or risk stratified based on its DNA and/or transcript expression. In some embodiments, a breast cancer is stratified based on risk utilizing a statistical model. Molecular classifications, in accordance with some embodiments, indicate the aggressiveness and risk of relapse. In some embodiments, integrative cluster (IntClust) subtype is used to molecularly classify a breast cancer. In various embodiments, copy number and/or transcript expression analysis of a set of one or more genes are used to classify a breast cancer into molecular pathology subgroups. Based on molecular pathology and/or risk stratification, a number of embodiments determine a course of treatment for a breast cancer, which may include measures to mitigate cancer recurrence and/or promote tumor shrinkage.

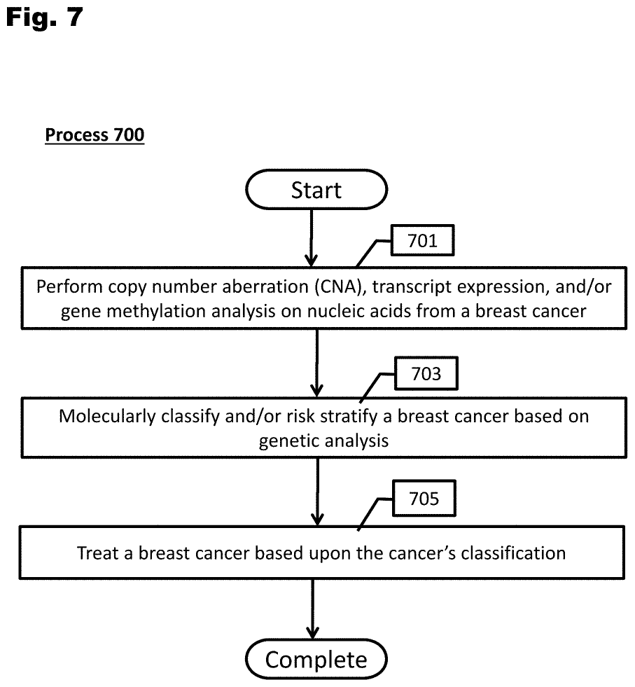

Provided in FIG. 7 is an embodiment of a method to molecularly classify and/or risk stratify a breast cancer. Process 700 begins with performing (701) copy number aberration (CNA) transcript expression and/or gene methylation analysis on nucleic acids from a breast cancer. In several embodiments, DNA and/or RNA transcripts are extracted from an individual having breast cancer and processed for analysis. DNA can be used to detect CNAs and/or methylation analysis at various genomic loci and RNA can be used to determine expression levels of various genes.

CNAs can be detected by a number of methods as described herein. In some embodiments, DNA of a cancer is extracted from an individual and processed to detect CNA levels. In various embodiments, RNA of a cancer is extracted and processed to detect expression levels of a number of genes. In some instances, gene expression is used directly for further analysis. In some instances, gene expression is utilized to determine whether aberrations in copy number impact expression and/or to delineate driver genes in a given patient's tumor. In some instances, CNA levels can be inferred from RNA sequencing data. Methylation of genes and/or determination of chromatin availability can be performed, which can be used for further analysis.

Nucleic acids can be extracted from a cancer biopsy and/or from an individual's bodily fluids (e.g., blood, plasma), including circulating tumor DNA (ctDNA), by a number of methodologies, as understood by practitioners in the field. Once extracted, nucleic acids can be processed and prepared for detection, as described herein. Methods of detection include (but are not limited to) hybridization techniques (e.g., in situ hybridization (ISH)), nucleic acid amplification techniques (e.g., PCR), and sequencing (e.g., exome, genome sequencing).

Genomic loci and/or genes are detected in accordance with various embodiments as described herein. In some embodiments, a set of probes and/or primers are used to identify a particular set of genomic CNAs and/or expressed transcripts. In various embodiments, whole or partial genomes, exomes, and/or transcriptomes are sequenced and analyzed to identify a particular set of genomic CNAs and/or expressed transcripts. In many embodiments, a particular set of genomic CNAs and/or expressed transcripts represent a particular molecular classification. In some embodiments, a molecular classification signifies a cancer's aggressiveness and risk of relapse. In some embodiments, a molecular classification signifies a cancer's molecular pathology. In some embodiments, a particular set of genomic CNAs and/or expression of transcripts represent a particular IntClust subgroup. In some embodiments, molecular classification is further used to stratify risk of recurrence.

Process 700 molecularly classifies and/or risk stratifies (703) a breast cancer based on genetic analysis (e.g., CNA, transcript expression, methylation analysis). In various embodiments, molecular class prediction models include (but are not limited to) shrinkage based methods, logistic regression, support vector machines with a linear kernel, support vector machines with a gaussian kernel, and neural networks. In various embodiments, statistical computation models include (but are not limited to) multi-state semi-markov Models, Cox Proportional Hazards models, shrinkage based methods, tree based methods, Bayesian methods, kernel based methods and neural networks.

The copy number amplifications described for the various IntClust subgroups, in accordance with various embodiments, are used as biomarkers for classifying a cancer into a particular subgroup as described herein. A number of embodiments utilize a previously trained computational classifier to assign a breast cancer into a particular molecular pathology subgroup (e.g., IntClust) as described herein. Various embodiments can utilize a previously trained risk stratification model to determine the risk of recurrence of a breast cancer. Accordingly, a computational classifier can utilize copy number features, gene expression features, genomic methylation features, and/or nucleosome occupancy features derived from DNA and RNA analysis of an individual having breast cancer. In some embodiments, copy number features are matched by either genomic position or gene name. In various embodiments, expression features are matched to a probe that detects expression and/or sequencing results. After features are matched, various embodiments scale each feature to a z-score and may include other normalization methods. In numerous embodiments, the matched features are entered into a molecular classifier and/ or risk stratification model to reveal how to treat an individual based on the molecular classification and/or risk of recurrence.

Process 700 also treats (705) a breast cancer based upon the cancer's molecular classification and/or risk stratification. In some embodiments, cancers classified into aggressive and/or late relapsing (e.g. IntClust subgroups 1, 2, 6 and 9) and/or high risk subgroups, a prolonged hormone/endocrine therapy (e.g., fulvestrant, anastrozole, exemestane, letrozole, tamoxifen, GDC9545) may be applied. In various embodiments, cancers classified into aggressive and/or late relapsing and/or high risk subgroups are treated with chemotherapy.

As previously noted, various IntClust subgroups are characterized by specific molecular aberrations and genomic drivers some of which can readily be therapeutically targeted. In some embodiments, IntClust1 cancers are treated with mTOR pathway antagonists (e.g., everolimus, temsirolimus, sirolimus, rapamycin), AKT1 antagonists (e.g., ipatasertib, capivasertib (AZD5363)), AKT1/RPS6KB1 antagonists (e.g., M2698), RPS6KB1 antagonists (e.g., LY2584702), PI3K antagonists (e.g., alpelisib, buparlisib (BKM120), pictilisib (GDC-0941)), elF4A antagonists (e.g., zotatifin), elF4E antagonists (e.g., rapamycin, rapamycin analogues, ribavirin, AZD8055),or a combination thereof. In various embodiments, IntClust2 cancers are treated with epigenetically targeted therapies, CDK4/6 antagonists (e.g., palbociclib, ribociclib, abemaciclib), FGFR pathway antagonists (e.g., lucitanib, dovitinib, AZD4547, erdafitinib, Infigratinib (BGJ398), BAY-1163877, Ponatinib), PARP-inhibitors (e.g., niraparib, olaparib), homologous recombination deficiency (HRD)-targeted therapies, PAK1 inhibitors (e.g., IPA3), elF4A antagonists (e.g., zotatifin), elF4E antagonists (e.g., rapamycin, rapamycin analogues, ribavirin, AZD8055), or a combination thereof. In some embodiments, IntClust6 cancers are treated with FGFR pathway antagonists (e.g., lucitanib, dovitinib, AZD4547, erdafitinib, Infigratinib (BGJ398), BAY-1163877, Ponatinib), elF4A antagonists (e.g., zotatifin), elF4E antagonists (e.g., rapamycin, rapamycin analogues, ribavirin, AZD8055), or a combination thereof. And in various embodiments, IntClust9 cancers are treated with selective estrogen receptor degraders (SERDs) (e.g., fulvestrant, GDC-9545, SAR439859 (SERD '859), RG6171, AZD9833), the proteolysis targeting chimera (PROTAC) ARV-471, SRC3 antagonists (e.g., SI-2), MYC antagonists (e.g., omomyc), BET bromodomain antagonists (e.g., JQ1, PROTAC ARV-771), elF4A antagonists (e.g., zotatifin), elF4E antagonists (e.g., rapamycin, rapamycin analogues, ribavirin, AZD8055), or a combination thereof.

While specific examples of processes for treating a breast cancer based upon molecular classification and/or risk stratification are described above, one of ordinary skill in the art can appreciate that various steps of the process can be performed in different orders and that certain steps may be optional according to some embodiments of the invention. As such, it should be clear that the various steps of the process could be used as appropriate to the requirements of specific applications. Furthermore, any of a variety of processes for treating a breast cancer to the requirements of a given application can be utilized in accordance with various embodiments of the invention.

### Methods of Treatment

Various embodiments are directed to treatments of breast cancer based on molecular characterization and/or risk stratification of the cancer. As described herein, classification of a breast cancer by the molecular pathology and/or the aggressiveness and risk of relapse of the cancer. Based on the classification, a breast cancer (or individuals having breast cancer) can be treated accordingly.

Several embodiments are directed to the use of medications to treat a breast cancer based on molecular classification and/or risk stratification of the cancer. In some embodiments, medications are administered in a therapeutically effective amount as part of a course of treatment. As used in this context, to “treat” means to ameliorate at least one symptom of the disorder to be treated or to provide a beneficial physiological effect. For example, one such amelioration of a symptom could be reduction of tumor size and/or risk of relapse.

A therapeutically effective amount can be an amount sufficient to prevent reduce, ameliorate or eliminate the symptoms of breast cancer. In some embodiments, a therapeutically effective amount is an amount sufficient to reduce cancer growth in a breast cancer growth, which can be determined by a number of ways including (but not limited to) measuring tumor size and measuring proliferation levels (e.g., Ki6730 expression).

A number of treatments and medications are available to treat breast cancer including (but not limited to) radiotherapy, chemotherapy, targeted (molecular) therapy, endocrine therapy, and immunotherapy. Accordingly, an individual may be treated, in accordance with various embodiments, by a single medication or a combination of medications described herein.

Classes of anti-cancer or chemotherapeutic agents can include alkylating agents, platinum agents, taxanes, vinca agents, anti-estrogen drugs, aromatase inhibitors, ovarian suppression agents, endocrine/hormonal agents, bisphosphonate therapy agents and targeted biological therapy agents. Medications include (but are not limited to) cyclophosphamide, fluorouracil (or 5-fluorouracil or 5-FU), methotrexate, thiotepa, carboplatin, cisplatin, taxanes, paclitaxel, protein-bound paclitaxel, docetaxel, vinorelbine, tamoxifen, raloxifene, toremifene, fulvestrant, gemcitabine, irinotecan, ixabepilone, temozolomide, topotecan, vincristine, vinblastine, eribulin, mutamycin, capecitabine, capecitabine, anastrozole, exemestane, letrozole, leuprolide, abarelix, buserelin, goserelin, megestrol acetate, risedronate, pamidronate, ibandronate, alendronate, zoledronate, and tykerb. Anthracyclines include (but are not limited to) daunorubicin, doxorubicin, epirubicin, idarubicin, valrubicin and mitoxantrone.

Endocrine therapy includes (but is not limited to) selective estrogen receptor modulators (SERMs), selective estrogen receptor degraders (SERDs), aromatase inhibitors, and PROTAC ARV-471. SERMs include (but are not limited to) tamoxifen, toremifene, raloxifene, ospemifene, and bazedoxifene. SERDs include (but are not limited to) fulvestrant, brilanestrant (GDC-0810), elacestrant, GDC-9545, SAR439859 (SERD '859), RG6171, and AZD9833. Aromatase inhibitors include (but are not limited to) anastrozole, exemestane, letrozole, vorozole, formestane, and fadrozole. Endocrine therapy for premenopausal women includes (but is not limited to) administration of tamoxifen, a SERD or an aromatase inhibitor. Ovarian ablation and/or ovarian suppression can also be performed. Endocrine therapy for postmenopausal women includes (but is not limited to) administration of SERM or an aromatase inhibitor.

Dosing and therapeutic regimes can be administered appropriate to the breast cancer to be treated, as understood by those skilled in the art. For example, anthracyclines can be administered intravenously at dosages from 10 mg/m² to 300 mg/m² per week. Likewise, 5-FU can be administered intravenously at dosages between 25 mg/m² and 1000 mg/m². Methotrexate can be administered intravenously at dosages between 1 mg/m² and 500 mg/m².

Any appropriate breast cancer can be treated, including Stage I, II, III, and IV breast cancer. Breast cancer with positive and/or negative status for estrogen receptor (ER), progesterone receptor (PR) and human epidermal growth factor 2 (Her2) can also be treated in accordance with various embodiments of the invention.

### Targeted Therapy Based Upon Oncogenic Pathology

Several embodiments are directed towards targeted (molecular) therapy to treat a breast cancer. In many of these embodiments, a targeted therapy is a therapy that specifically targets the molecular pathology or oncogenic driver of a breast cancer, which is determined based upon molecular classification (e.g., classification into an IntClust subgroup). Accordingly, a targeted therapy is one that mitigates the function of the oncogenic drivers, such as (for example) antagonists that inhibit the activity of the oncogenic driver. In some embodiments, a targeted therapy targets the pathway of the oncogenic driver. In some embodiments, a companion diagnostic is utilized to determine whether to utilize a targeted therapy in which the companion diagnostic identifies an oncogenic driver of the breast cancer.

It is now appreciated that ER+/HER2− breast cancers that classify within IntClust subgroups 1, 2, 6 and 9 are a more aggressive cancer with a high likelihood to relapse. It is further appreciated that the oncogenic drivers of the high risk subgroups can be targeted such to improve therapies to this hard to treat group. As shown in FIGS. 3A and 3B, some oncogenic drivers of IntClust1 are RPS6KB1, PRR11, and/or BCAS3 some oncogenic drivers of IntClust2 are FGF3/FGF4/FGF19, CCND1 likely in combination with EMSY, PAK1 and/or RSF1, some oncogenic drivers of IntClust6 are FGFR1, EIF4EBP1, and/or ZNF703, and an oncogenic driver of IntClust9 is MYC and/or NCOA3.

In several embodiments, oncogenic pathologies are targeted directly. In some embodiments, IntClust1 cancers are treated with mTOR pathway antagonists (e.g., everolimus, temsirolimus, sirolimus, rapamycin), AKT1 antagonists (e.g., ipatasertib, capivasertib (AZD5363)), AKT1/RPS6KB1 antagonists (e.g., M2698), RPS6KB1 antagonists (e.g., LY2584702), PI3K antagonists (e.g., alpelisib, buparlisib (BKM120), pictilisib (GDC-0941)), elF4A antagonists (e.g., zotatifin), elF4E antagonists (e.g., rapamycin, rapamycin analogues, ribavirin, AZD8055),or a combination thereof. In various embodiments, IntClust2 cancers are treated with epigenetically targeted therapies, CDK4/6 antagonists (e.g., palbociclib, ribociclib, abemaciclib), FGFR pathway antagonists (e.g., lucitanib, dovitinib, AZD4547, erdafitinib, Infigratinib (BGJ398), BAY-1163877, Ponatinib), PARP-inhibitors (e.g., niraparib, olaparib), homologous recombination deficiency (HRD)-targeted therapies, PAK1 inhibitors (e.g., IPA3), elF4A antagonists (e.g., zotatifin), elF4E antagonists (e.g., rapamycin, rapamycin analogues, ribavirin, AZD8055), or a combination thereof. In some embodiments, IntClust6 cancers are treated with FGFR pathway antagonists (e.g., lucitanib, dovitinib, AZD4547, erdafitinib, Infigratinib (BGJ398), BAY-1163877, Ponatinib), elF4A antagonists (e.g., zotatifin), eIF4E antagonists (e.g., rapamycin, rapamycin analogues, ribavirin, AZD8055), or a combination thereof. And in various embodiments, IntClust9 cancers are treated with selective estrogen receptor degraders (SERDs) (e.g., fulvestrant, GDC-9545, SAR439859 (SERD '859), RG6171, AZD9833), the proteolysis targeting chimera (PROTAC) ARV-471, SRC3 antagonists (e.g., SI-2), MYC antagonists (e.g., omomyc), BET bromodomain antagonists (e.g., JQ1, PROTAC ARV-771), elF4A antagonists (e.g., zotatifin), elF4E antagonists (e.g., rapamycin, rapamycin analogues, ribavirin, AZD8055), or a combination thereof.

### Stratification and Treatments for Early Stage ER+/HER2− Breast Cancer

A number of embodiments are directed towards methods of treatments of early stage breast cancer in which IntClust classification and/or risk stratification is utilized to stratify treatment. In current protocol standards, a breast cancer screening provides some preliminary determinations on how to proceed. Typically, basic histology and tumor assessment, and imaging is performed, including determining cancer stage (i.e., Stages I, II, III, and IV tumor type (i.e., ductal, lobular, mixed, metaplastic), tumor size, presence of cancer within lymph nodes, and basic genetic analysis (i.e., status of progesterone receptor (PR), estrogen receptor (ER), and human epidermal growth factor receptor 2 (HER2). Based on these factors, particular treatments are performed, as currently practiced in the field.

When an ER+/HER2− breast cancer is Stage I to III and node negative, it is considered an early stage breast cancer. In accordance with current standards of care, early stage ER+/HER2− breast cancer that has a tumor less than 0.5 cm is treated with surgery and adjuvant endocrine therapy. When an early stage ER+/HER2− breast cancer has a tumor greater than 0.5 cm, in accordance with current standards of care, molecular testing is often performed, such as Oncotype DX, to determine risk of recurrence. When risk of recurrence is low (e.g., Oncotype score<18), treatment entails surgery and adjuvant endocrine therapy. When risk of recurrence is high (e.g., Oncotype score≥31), treatment entails surgery, adjuvant endocrine therapy, and adjuvant chemotherapy. When risk of recurrence is intermediate (e.g., Oncotype score 18-30), treatment entails surgery and adjuvant endocrine therapy with the possibility to also perform adjuvant chemotherapy. The benefit of adjuvant chemotherapy in the intermediate risk is not clear, due to lack of stratification of risk within this group.

In a number of embodiments, IntClust classification is to be used as a molecular test on an early stage ER+/HER2− breast cancer, whether or not it is node positive or negative. Accordingly, in some embodiments, early stage ER+/HER2− breast cancer is classified into a high risk IntClust subgroup (i.e., IntClust subgroups 1, 2, 6 or 9) is treated with surgery, adjuvant endocrine therapy, and adjuvant chemotherapy. In some embodiments, IntClust classification is used as a feature within a statistical model to determine risk of recurrence. In some embodiments, a cancer stratified as high risk or classified into a high risk IntClust subgroup, receives targeted therapy directed at the molecular drivers of an IntClust subgroup. And in some embodiments, early stage ER+/HER2− breast cancer stratified as lower risk or classified into a lower risk IntClust subgroup (i.e., IntClust subgroups 3, 4ER+, 7 or 8) is treated with surgery and adjuvant endocrine therapy, but not chemotherapy to reduce the harmful effects associated with chemotherapy.

In a number of embodiments, risk stratification and/or IntClust classification is used in addition to a classical molecular test on an early stage ER+/HER2− breast cancer. In some embodiments, risk stratification and/or IntClust classification is used when risk of recurrence is determined to be intermediate by another model (e.g., Oncotype score 18-30) to further stratify these patients. Accordingly, in some embodiments, when an early stage ER+/HER2− breast cancer is classified into an intermediate risk group by classical methods (e.g., Oncotype score 18-30) and high risk by methods described herein, (e.g., molecular classification into a high risk IntClust subgroup), the cancer is treated with surgery, adjuvant endocrine therapy, and adjuvant chemotherapy. In some embodiments, a cancer stratified as high risk also receives targeted therapy directed at the molecular drivers of an IntClust subgroup. And in some embodiments, when an early stage ER+/HER2− breast cancer is classified into an intermediate risk group by classical methods (e.g., Oncotype score 18-30) and a lower risk by methods described herein, (e.g., molecular classification into a lower risk IntClust subgroup) is treated with surgery and adjuvant endocrine therapy, but not chemotherapy.

It is noted that the classification of molecular test scores such as Oncotype into low, intermediate and high may change (See, e.g., J. A. Sparano, et al., N. Engl. J. Med. 379, 111-121 (2018), the disclosure of which is herein incorporated by reference). Despite the changes that may occur, the proposition of using a molecular driver classification (e.g., IntClust classification) to better comprehend scores still applies. As detailed in the Exemplary Embodiments, the utilization of a molecular driver classification in combination with Oncotype yields a better comprehension of risk of relapse than Oncotype alone.

Several other molecular classification assessments can be performed on early stage breast cancer, including Prosigna, MammaPrint, EndoPredict, BCI. Accordingly, in several various embodiments, IntClust classification is used in addition to Prosigna, MammaPrint, EndoPredict, BCI, or a combination thereof. In many embodiments, IntClust classification can be combined with another molecular classification to confirm a diagnosis and/or better stratify patients to determine an appropriate treatment strategy.

Menopausal status of women can also be helpful in determining appropriate treatment, as the regulation of estrogen is important. For pre-menopausal women with ER+/HER2− breast cancer and higher risk of recurrence (young age, high-grade tumor, lymph node involvement or based on molecular predictors of risk of recurrence), tamoxifen or an aromatase inhibitor (plus ovarian suppression or ablation) for 5 years is administered in accordance with some embodiments. Aromatase inhibitors include (but are not limited to) anastrozole, exemestane, and letrozole.

In a number of embodiments, for postmenopausal women, tamoxifen is administered for 4.5-6 years and up to 10 years. In some embodiments, aromatase inhibitors are administered to postmenopausal women. As part of their treatment plan, some post-menopausal women will use aromatase inhibitors alone in accordance with various embodiments. Others will use tamoxifen for 1-5 years and then begin using aromatase inhibitors in accordance with various embodiments. Aromatase inhibitors include (but are not limited to) anastrozole, exemestane, and letrozole.

A number of embodiments utilize a targeted treatment for early stage breast cancer. For instance, in some embodiments, early stage breast cancers having RPS6KB1 oncogenic pathologies (e.g., IntClust1), capivasertib (AZD5363) or M2698 can be administered. In one treatment regimen, capivasertib is administered at 400 mg twice daily (2 oral tablets) given on an intermittent weekly dosing schedule with 4 days on and 3 days off (i.e., dosed on Days 2 to 5 of Weeks 1, 2, and 3 followed by 1 week off-treatment within each 28-day treatment cycle). It may be given in combination with endocrine therapy such as fulvestrant (500 mg) and potentially with tamoxifen. M2698 can be administered at 240 mg daily alone or at 160 mg daily in combination with tamoxifen. In cancers that have the FGFR pathway oncogenic pathologies (e.g., FGFR and FGF oncogenes) (e.g., IntClust2, IntClust6), infigratinib can be administered at 75-125 mg daily, 3 weeks on, 1 week off. In cancers that have the CDK4/6 oncogenic pathologies (e.g., IntClust2, IntClust6), palbociclib can be administered at 125 mg daily, 3 weeks on, 1 week off.

While specific treatment regimens are described, these are provided as exemplary treatment options. It should be understood that alterations of dosing amount, and/or schedule are to be included within various embodiments. It should also be understood that various treatment combinations can be altered, substituted, and/or combined with other treatment combinations, as would be appreciated by those skilled in the art. For example, various treatment regimens including fulvestrant can be altered to include other SERDs, tamoxifen or an aromatase inhibitor. Because fulvestrant has low oral availability, in some embodiments, PROTAC ARV-471 or an orally available SERD such as GDC-9545, SAR439859 (SERD '89), RG6171, or AZD9833, may be utilized.

### Treatments for Metastatic ER₊/HER2− Breast Cancer

A number of embodiments are directed towards methods of treatments of metastatic breast cancer in which IntClust classification is utilized. In current protocol standards, a breast cancer screening provides some preliminary determinations on how to proceed. Typically, basic histology and tumor assessment is performed, including determining cancer stage (i.e., Stages I, II, III, and IV tumor type (i.e., ductal, lobular, mixed, metaplastic), tumor size, presence of cancer within lymph nodes, and basic genetic analysis (i.e., status of progesterone receptor (PR), estrogen receptor (ER), and human epidermal growth factor receptor 2 (HER2). Based on these factors, particular treatments are performed, as currently practiced in the field.

When an ER+/HER2− breast cancer is Stage IV and/or node positive, it is considered a metastatic breast cancer. Treatment determination depends on whether the woman is premenopausal or postmenopausal. For premenopausal women, treatment includes (but is not limited to) administration of tamoxifen, toremifene, or fulvestrant. Ovarian ablation and/or ovarian suppression can also be performed. For postmenopausal women, treatment includes (but is not limited to) administration of tamoxifen and/or an aromatase inhibitor. These treatments can be performed from 5 years, up to 10 years.

In a number of embodiments, metastatic cancer is administered a targeted treatment. For instance, in some embodiments, cancers having RPS6KB1 oncogenic pathologies (e.g., IntClust 1), capivasertib (AZD5363) or ipatasertib can be administered and can be combined with an aromatase inhibitor and/or other endocrine therapy. A number of treatment regimens are contemplated. In one regimen, treatment includes capivasertib and an aromatase inhibitor, and capivasertib is administered 4 days on 3 days off at 400 mg/day, while aromatase inhibitors will be administered on a daily basis. In one regimen, treatment includes capivasertib and fulvestrant, and capivasertib is administered 4 days on 3 days off at 400 mg/day, while 500mg of fulvestrant will be administered on day 1 and 15 of a 28-day cycle and again on day 1 each subsequent cycle. In one regimen, treatment includes capivasertib and fulvestrant and palbociclib, and capivasertib is administered 4 days on 3 days off at 400 mg/day, while 500 mg of fulvestrant will be administered on day 1 and 15 of a 28-day cycle and again on day 1 each subsequent cycle and palbociclib will be administered orally on a 3 week on and 1 week off schedule. In one regimen, treatment includes ipatasertib and an aromatase inhibitor, and ipatasertib is administered daily at 400 mg/day along with aromatase inhibitors that will also be administered on a daily basis. In one regimen, treatment includes ipatasertib and fulvestrant, and ipatasertib is administered at 400 mg/day daily, while 500 mg of fulvestrant will be administered on day 1 and 15 of a 28-day cycle and again on day 1 each subsequent cycle. In one regimen, treatment includes ipatasertib and fulvestrant and palbociclib, and ipatasertib is administered at 400 mg/day daily, while 500 mg of fulvestrant is administered on day 1 and 15 of a 28-day cycle and again on day 1 each subsequent cycle and palbociclib will be administered orally on a 3 week on and 1 week off schedule.

In a number of embodiments, metastatic cancer is administered a targeted treatment, which can be determined by IntClust classification. For instance, in some embodiments, cancers having FGFR pathway (e.g., FGFR and/or FGF oncogenes) oncogenic pathologies (e.g., IntClust2, IntClust6), infigratinib (BGJ398) can be administered and can be combined with an aromatase inhibitor and/or other endocrine therapy or potentially chemotherapies. A number of treatment regimens are contemplated. In one regimen, treatment includes infigratinib and an aromatase inhibitor, and infigratinib is administered daily at 125 mg/day for 3 weeks on and 1 week off, while Als that will be administered on a daily basis. In one regimen, treatment includes infigratinib and fulvestrant, and infigratinib is administered at 125 mg/day daily for 3 weeks on and 1 week off, while 500 mg of fulvestrant will be administered on day 1 and 15 of a 28-day cycle and again on day 1 each subsequent cycle. In one regimen, treatment includes infigratinib and fulvestrant and palbociclib, and infigratinib administered at 125 mg/day daily 3 weeks on and 1 week off, while 500 mg of fulvestrant is administered on day 1 and 15 of a 28-day cycle and again on day 1 each subsequent cycle and palbociclib will be administered orally on a 3 week on and 1 week off schedule.

While specific treatment regimens are described, these are provided as exemplary treatment options. It should be understood that alterations of dosing amount, and/or schedule are to be included within various embodiments. It should also be understood that various treatment combinations can be altered, substituted, and/or combined with other treatment combinations, as would be appreciated by those skilled in the art. For example, treatment regimens inclusive of palbociclib can be altered to include ribociclib and/or abemaciclib.

### Treatments for Triple Negative Breast Cancer

A number of embodiments are directed towards methods of treatments of triple negative cancer in which IntClust classification is utilized. In current protocol standards, a breast cancer screening provides some preliminary determinations on how to proceed. Typically, basic histology and tumor assessment is performed, including determining cancer stage (i.e., Stages I, II, III, and IV tumor type (i.e., ductal, lobular, mixed, metaplastic), tumor size, presence of cancer within lymph nodes, and basic genetic analysis (i.e., status of progesterone receptor (PR), estrogen receptor (ER), and human epidermal growth factor receptor 2 (HER2). Based on these factors, particular treatments are performed, as currently practiced in the field.

When a breast cancer lacks amplification of PR, ER, or HER2 (i.e., PR−, ER−, and HER2−), it is considered a triple negative breast cancer. For triple negative breast cancer (TNBC), therapies that target hormones or HER2 do not work. Instead, in accordance with current standards of care, TNBC is treated with a combination of surgery, radiation therapy and/or chemotherapy. An emerging option for TNBC is treatment with checkpoint inhibitors such as pembrolizumab or nivolumab and/or immunotherapies that target the protein PD-L1 or PD1 such as atezolizumab (Tecentriq). In some embodiments, TNBCs that classify within IntClust4ER− are treated with atezolizumab, as cancers within this classification have a high degree of immune infiltration and a persistent risk of recurrence. In some embodiments, TNBCs that classify within IntClust10 are treated with atezolizumab after or potentially in combination with radiation or chemotherapy to better stimulate the immune system and thus more sensitive to the atezolizumab treatment.

### Patient Derived Organoid Development and Use

Several embodiments are directed to the development and use of patient derived organoids (PDOs), which are three-dimensional tissue of cancer cell derived from a patient's cancer tissue and cultured in vitro, where oncogenic signaling in three-dimensional cultures better mimic the in vivo setting. PDOs can also be xenotransplanted in vivo. PDOs recapitulate the biological features of a patient's cancer and thus are well-suited models to investigate the ability of drug compounds to treat a cancer. In addition, PDOs can be developed for the high risk breast cancers, which are not well represented amongst existing cancer cell lines.

In various embodiments, PDO lines are developed for general and/or personal drug compound treatment investigation. Accordingly, in some embodiments, a PDO line is characterized into a molecular subgroup (e.g., an IntClust subgroup) and utilized as model to infer candidate drug compounds to treat patients that fall within that subgroup. In some embodiments, a panel of PDO lines with a molecular subgroup are investigated to infer candidate drug compounds to treat patients that fall within that subgroup. And in some embodiments for personalized assessment, PDO lines are derived from a particular patient and then assessed to infer which drug compounds to treat that patient.

For general drug compound treatment investigation, an embodiment of a method to infer candidate drug compounds can be performed as follows:
Extract cancer cells from one or more patients
Classify the oncogenic pathology of the tissue into a molecular subgroup
Develop a panel of one or more PDO lines from one or more patients; each PDO line within the panel sharing a similar molecular pathology (e.g., a panel of PDO lines within an IntClust subgroup)
Administer drug compounds on the panel to identify candidate drug compounds for treatment of patients sharing the similar molecular pathology

In some embodiments, results of a general drug compound treatment investigation are utilized as pre-clinical data or to develop a clinical trial on patients. In some embodiments, compound concentration is assessed (e.g., IC₅₀). In some embodiments, compound toxicity on cancer cells is assessed. In some embodiments, compound toxicity on healthy cells is assessed to determine potential off-target and/or side effects.

For personal drug compound treatment investigation, an embodiment of a method to infer candidate drug compounds can be performed as follows:
Extract cancer cells from a patients
Optional: characterize the patient's cancer or derivative PDO into a molecular subgroup
Develop a panel of one or more PDO lines from the patient
Test drug compounds in the panel to identify drug compounds for a particular treatment regimen for the patient
   Optional: drug compounds to be tested are candidate compounds for a particular molecular subgroup
   Optional: test combinations of drug compounds to determine a more optimal combination of drugs for the treatment regimen

In some embodiments, results of a personal drug compound treatment investigation are utilized to administer a personal treatment on a patient. In some embodiments, compound concentration is assessed. In some embodiments, compound toxicity on a patient's cancer cells is assessed. In some embodiments, compound toxicity on a patient's healthy cells is assessed to determine potential off-target and/or side effects.

### EXEMPLARY EMBODIMENTS

The embodiments of the invention will be better understood with the several examples provided within. Many exemplary results of processes that identify molecular indicators of breast cancer relapse are described. Validation results are also provided.

### Example 1: Dynamics of Breast Cancer Relapse

Breast cancer has multiple stages of progression (i.e., a multistate disease), with clinically relevant intermediate endpoints such as recurrence in loco-regional or distant locations. These recurrence events are correlated, and individual survival analyses of one endpoint cannot fully capture patterns of recurrence that may be associated with differential prognosis. A patient's prognosis can differ dramatically depending on when and where a relapse occurs, time since surgery, and time since loco-regional or distant relapse. These distinct states and timescales are generally not accounted for and motivate the development of a unified statistical framework, as proposed here.

To overcome these limitations, various embodiments incorporate a computational model that accounts for different clinical endpoints and timescales, as well as competing risks of mortality, enabling a description of an individual's risk, including risk of relapse. In some of these embodiments, a non-homogenous (semi) Markov chain model is used. Application of these models to cohorts of breast cancer patients with years of clinical follow-up, including many patients with accompanying molecular data, can delineates the spatio-temporal dynamics of breast cancer relapse across distinct molecular subgroups. In particular, the patterns of relapse across the clinical subgroups, PAM50 subgroups (C. M. Perou, et al. Nature 406, 747-52 (2000), J. S. Parker J. Clin. Oncol. 27, 1160-67 (2009), the disclosures of which are each incorporated by reference in their entirety) and integrative clusters (IntClust) defined based on integration of genomic copy number alterations and transcriptional profiles (C. Curtis, et al., 2012, cited supra), were evaluated to identify molecular subgroups of patients having aggressive cancer and high risk of recurrence. Of note, in several embodiments, four Integrative Subgroups harboring specific genomic drivers have high risk of recurrence up to twenty years post initial diagnosis. These four subgroups were found to account for approximately 25% of all ER+ tumors. In addition, each of these four subgroups maps to one of the integrative clusters, and is enriched for a characteristic copy number amplification events of various sections of the genome, including 11q13 (FGF3, CCND1, RSF1), 8p12 (FGFR1, ZNF703), 17q23 (RPS6KB1), and 8q24 (MYC). The use of these integrated clusters was found to improve prediction of late distant relapse beyond standard clinical covariates, which is corroborated in an external validation cohort. It was also found that a subgroup of triple-negative breast cancer patients rarely recur after 5 years while others remain at risk. After distant recurrence, tumor subtype continues to dictate the rate of subsequent metastases, underscoring the importance of classifying tumors accordingly. Based on these findings, several embodiments are directed to identifying individuals having a particular risk of aggressive cancer and relapse, as determined by a diagnostic method. Various embodiments treat and/or monitor an individual based on their cancer aggressiveness and risk of relapse.

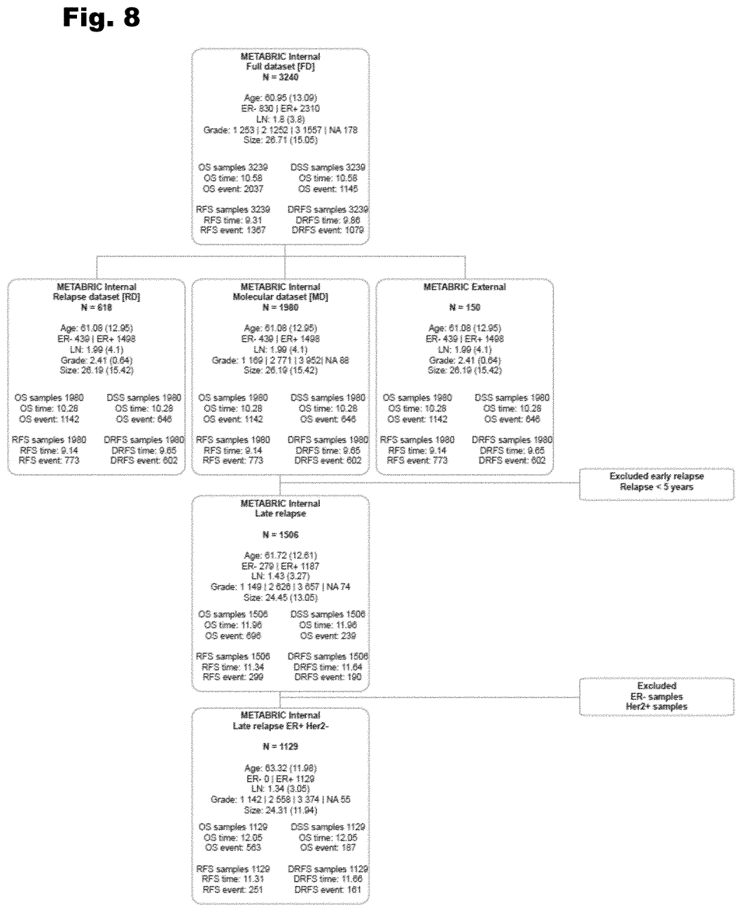

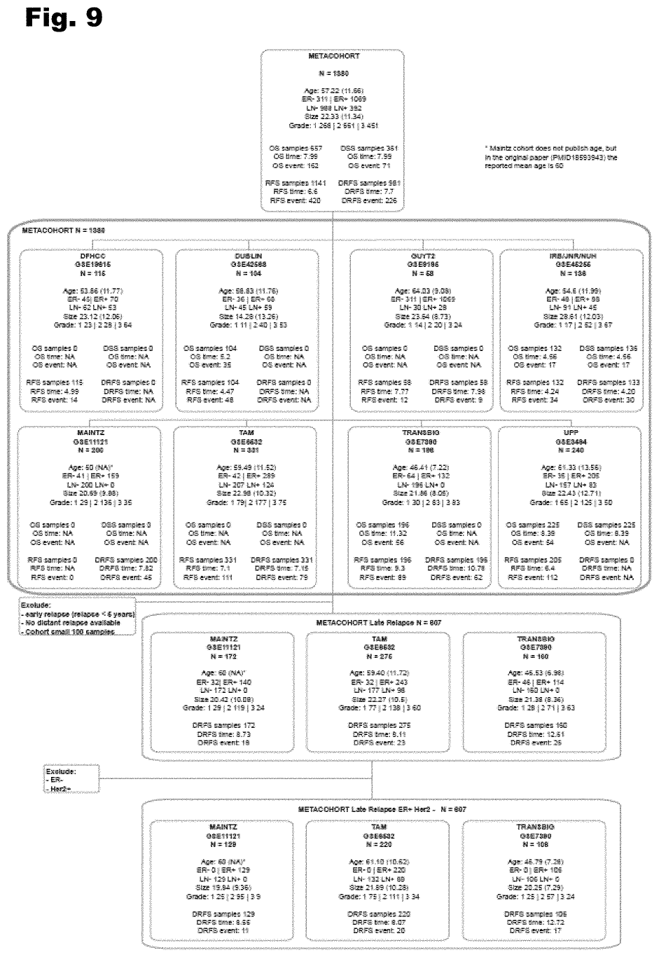

Data from 3,240 patients derived from five tumor banks in the UK and Canada was employed for studies described herein, referred to herein as the Full Dataset [FD] (median follow-up of 9.75 years). The [FD] included clinical and pathological variables and was used to define the clinical subtypes (ER+/HER2+, ER+/HER2−, ER−/HER2+, ER−/HER2-). For a subset of 1,980 patients an integrated genomic analysis based on gene expression and copy number data was previously described and referred to herein as the molecular dataset or METABRIC [MD] For this cohort, tumors are stratified based on clinical subtypes, intrinsic subtypes (PAM50) (C. M. Perou, et al., (2000), and J. S. Parker, et al., (2009) cited supra) and integrative cluster (IntClust) membership (C. Curtis, et al., (2012), and H. R. Ali, et al., (2014), cited supra). Finally, for a subset of patients who experienced distant metastasis (618 out of the 1079 who relapsed), full information on the dates of each recurrence (rather than only the first) is available, enabling analysis of spatio-temporal dynamics. This data is referred herein as the recurrent events dataset [RD]. These three datasets are summarized in Table 1 with clinical details provided in Tables 2-4, FIG. 8. An independent cohort composed of 1380 breast cancer patients was used to externally validate the findings (FIG. 9).

From the [FD] several basic parameters that naïvely describe the two key intermediate endpoints in breast cancer were derived: loco-regional relapse (LR) and distant relapse (DR). For this example, loco-regional relapse is a local or regional recurrence, including lesions in the same breast, skin of chest, axilla, internal mammary, axillary, or supraclavicular lymph nodes. A distant relapse is defined as a distant metastasis.

Among the 2297 ER+ patients, 312 (14%) and 718 (31%) patients experienced a LR or DR, respectively, and 176 (8%) had both LR and DR, whereas among the 850 ER− patients, 140 (16%) experienced LR, 335 (39%) experienced a DR and 111 (13%) had both. Amongst patients who recurred, the average time to relapse differed with ER+ patients averaging 5.7 years to LR and 5.4 years to DR, while ER+ patients averaged 2.8 years to LR and 2.8 years to DR. Finally, among those patients who experienced a LR, 56% of ER+ and 79% of ER− patients went on to have a DR or breast cancer death. The average time to DR or breast cancer death after LR was 2.1 years for patients with ER+ tumors and 0.9 years for those with ER− disease.

Basic quality control was performed on the data. Observations that had relapse times equal to zero or relapse times equal to the last observed time were shifted 0.1 days. Local relapses that occurred after distant relapses were omitted. Eleven cases with stage IV cancer were also omitted from analysis. Benign and phylloid tumors were removed from analysis. Last follow-up time or time of death was the final endpoint for all patients. Special care was taken to remove second primary tumors from the dataset. The total number of cases used in each model can differ due to different missing values in clinical variables, molecular classification, etc.

### A Multistate Model for Breast Cancer Recurrence

An analysis of survival incorporating the intermediate events of LR and DR was also examined. While most studies examine disease-free survival or overall survival, there are significant limitations to this approach. Importantly, ER+ patients experience higher mortality from non-malignant causes than ER− patients because they tend to be older at the time of diagnosis.

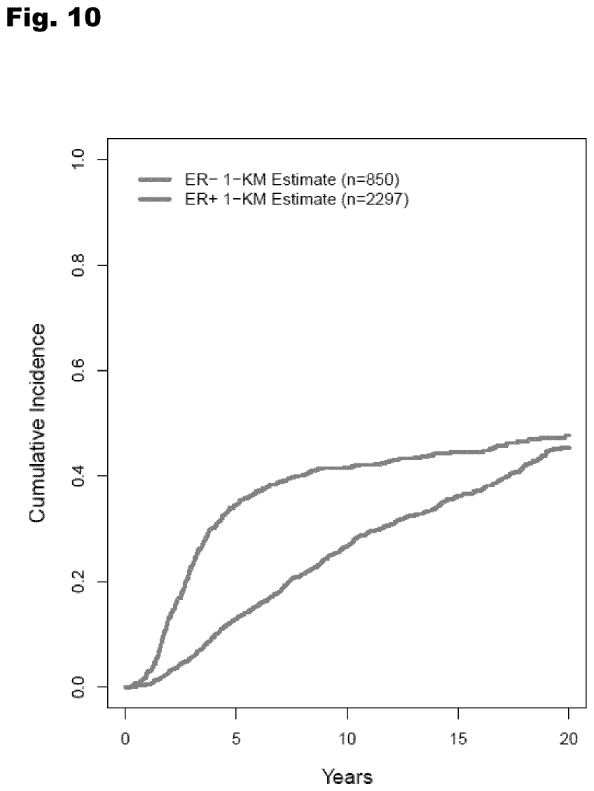

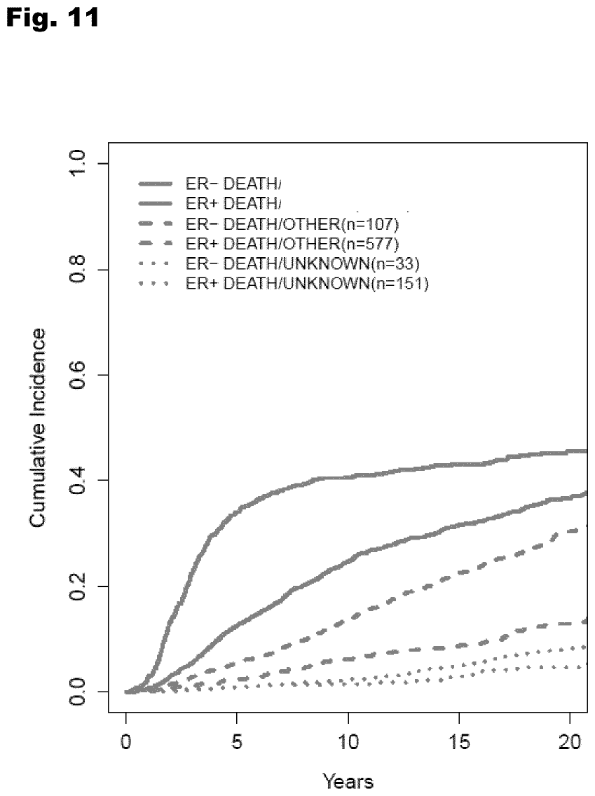

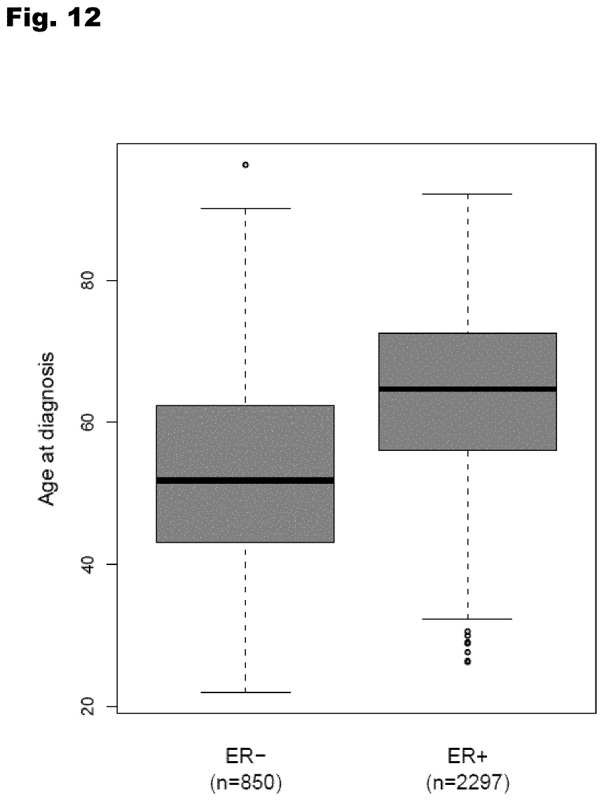

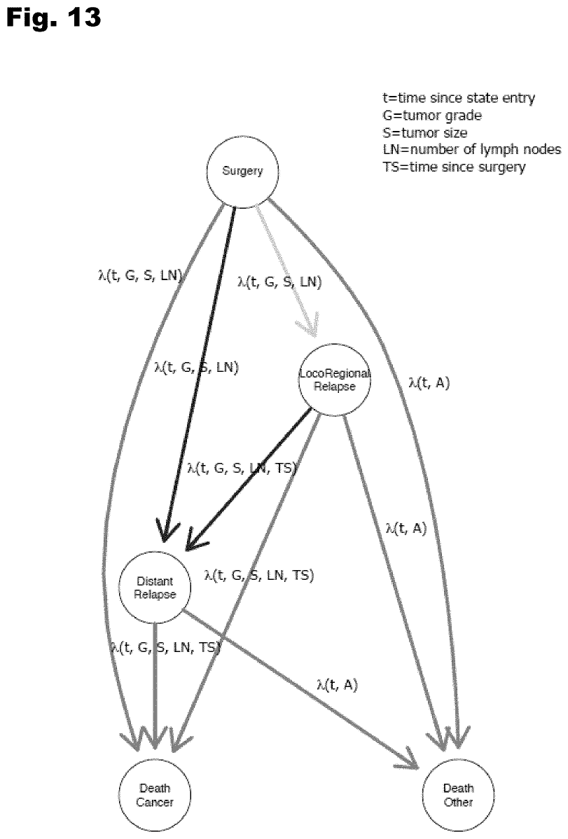

Most survival analyses employ disease-specific death as the primary endpoint and censor natural deaths, however, this strategy produces a censoring mechanism that is not independent of the variables studied in situations where several competing risks are present and resulting in a Kaplan-Meier estimate of survival that is biased. The extent of the bias in the cohort is evident by comparing the naïve cumulative incidence for cancer-related deaths (computed as 1—the survival probability) for ER− and ER+ patients taking into account only cancer-related deaths (FIG. 10) relative to the estimates with the proper cumulative incidence functions for different causes of death (FIG. 11). As described in this example, a cancer-related death is any death that has been labeled as cancer-related in the death certificate. If the cause of death was labeled as for another reason, unknown, or missing, the death was considered an “other” cause death. These comparisons indicate that the incidence of disease-specific death is overestimated for ER+ tumors (0.46 at 20 years vs 0.37). This is because the age of diagnosis is higher for ER+ than ER− tumors (median 63.9 vs 53.0 years; p-value<2.2e-16), and therefore patients have greater risk of non-malignancy related death (FIG. 12). Using overall survival as an endpoint does not resolve this issue, as it merges two different causes of death and inflates the risk in ER+ patients. Furthermore, since the baseline survival functions for pathological subgroups are distinct (FIG. 13), their differences cannot be adequately summarized with a single parameter in a Cox proportional hazards model.

To overcome these challenges, a statistical model was developed that accounts for different disease states (LR and DR), different timescales (time from diagnosis and time from relapse), competing causes of death (cancer death or other causes), clinical covariates or age effects, and distinct baseline hazards for different molecular subgroups (see H. Putter, M. Fiocco, & R. B. Geskus, Stat. Med. 26, 2389-430 (2007); O. Aalen, O. Borgan, & H. Gjessing, SURVIVAL AND EVENT HISTORY ANALYSIS—A PROCESS POINT OF VIEW. (Springer-Verlag New York, 2008); and T. M. Therneau & P. M. Grambsh, MODELING SURVIVAL DATA: EXTENDING THE COX MODEL. (Springer-Verlag New York, 2000); the disclosures of which are each cited supra). The multistate statistical model (FIG. 13) was fit to the [FD], thereby accounting for the chronology of breast cancer, starting with surgical excision of the primary tumor, followed by the development of loco-regional and/or distant recurrence and accounting by competing risks of death due to cancer or other causes. The hazards of occurrence of each of these states are modeled with a non-homogenous semi-Markov Chain with two absorbent states (Death/Cancer and Death/Other), and the number of transitions between each pair of states was recorded (Tables 5-7).

The model was stratified by molecular subtype and used a clock-reset time scale, in which the clock stops when the patient enters a new state. Although there were a small number of transitions from distant to local relapse (15 ER+ cases and 7 ER−), the local relapse was omitted in these instances as it was considered redundant and only allowed transitions from local to distant relapse in our model. The possibility of cancer death without a recurrence was included to account for cases where metastasis was not detected. The R packages mstate and survival were used to fit the data. For more on mstate and survival, see L. C. de Wreede, M. Fiocco, and H. Putter J. Stat Softw. 38, 1-30 (2011), the disclosure of which is herein incorporated by reference; and T.M. Therneau and P.M Grambsch, 2000, cited supra.

Several covariates were included in the model: age at diagnosis, tumor grade, tumor size, and the number of positive lymph nodes. Lymph nodes, which were entered as a continuous variable but capped at 10 lymph nodes to avoid influential observations from extreme cases. The time from diagnosis was also included as continuous.

The model employs independent baseline hazards for ER+ and ER− disease, in accordance with their distinct profiles. For dataset [FD], a Cox model was fitted stratified on ER status. Age had the same coefficient for all transitions into death/other causes for both ER values. Grade, Size and Lymph Nodes had different coefficients from the starting state to states of recurrence/death for each ER status. Time since diagnosis had different coefficients from the starting state of relapse to states of recurrence/death for each ER status and time since loco-regional relapse had different coefficients from distant relapse state to cancer related death for each ER status.

The majority of cancer related deaths (83% in ER+ and 87% in ER− tumors) occurred subsequent to distant metastasis (Table 5). The remainder of cases reflect either undetected recurrences or situations where patients succumbed to another malignancy.

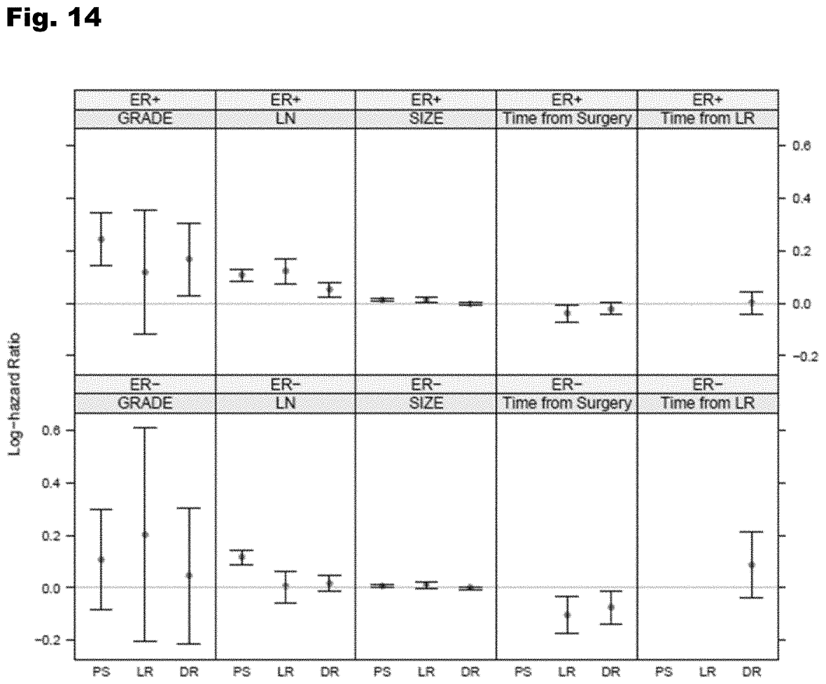

Age was significantly associated with the transition to death by other causes (p-value<0.01). Examination of the log hazard ratios and 95% confidence intervals for all other variables indicates that the effect of each variable decreased with disease progression (FIG. 14). This implies that clinical variables related to the primary tumor were more prognostic for earlier transitions (e.g., from a disease-free state to recurrence) than for later transitions (e.g., from DR to death). Several tumor characteristics, however, informed the risk of progression from LR to DR and from DR to death. In ER+ cancer, tumor grade, tumor size, and number of positive lymph nodes all increased the risk of progression to a “worse” state. A longer time between surgery and LR or between surgery and DR, however, decreased the risk of transition to a “worse” state, and this decreased risk was more prevalent in ER− cancer. The amount of time after LR was not predictive of the onset of DR. Hence, this variable was not included in the remainder of analyses.

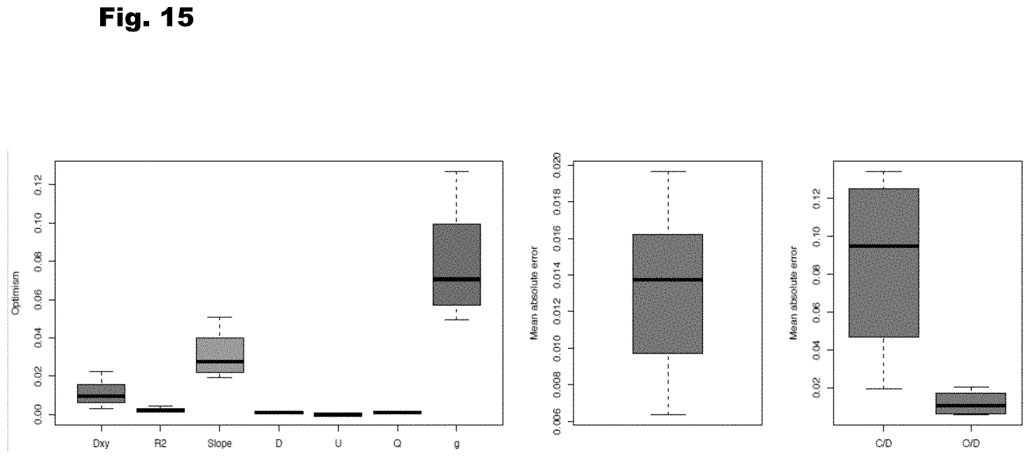

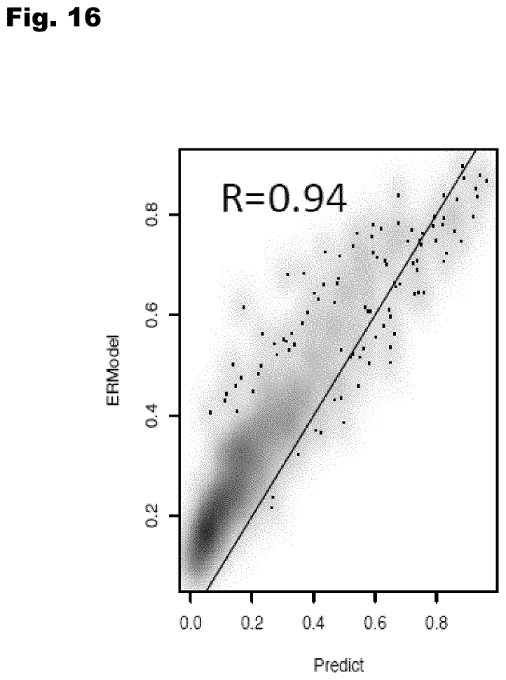

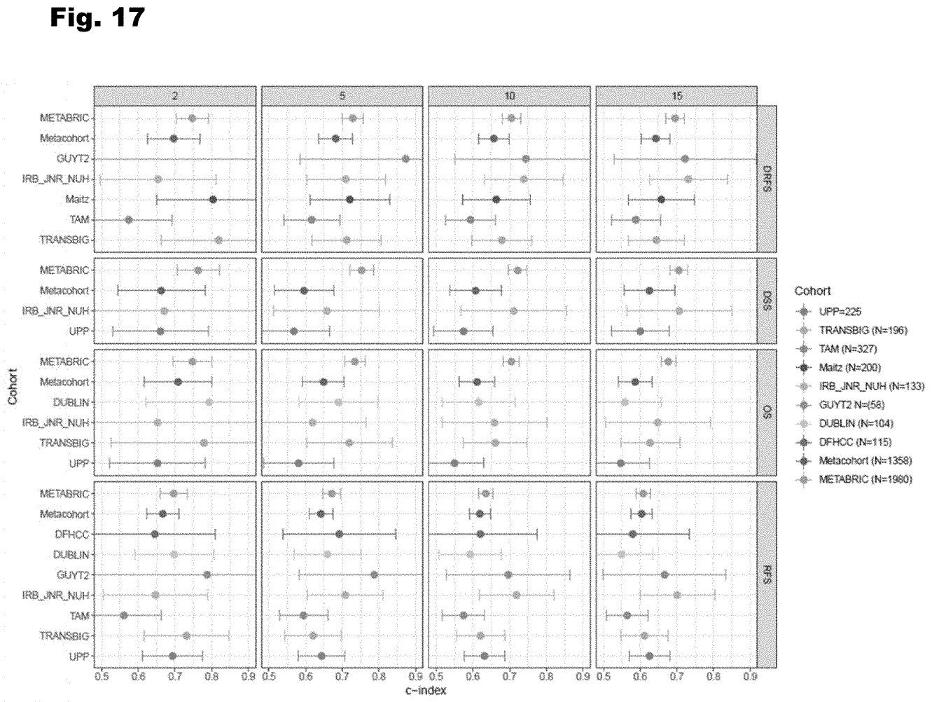

Extensive validation indicates that these models are well calibrated and not prone to overfitting (FIG. 15). Moreover, strong concordance is shown for a basic model stratified by ER status relative to the established tool Predict (FIG. 16) with comparable model performance in an external metacohort (FIG. 17) (for more on Predict, see G. C. Wishart, et al., Breast Cancer Res. 12, R1 (2010), the disclosure of which is herein incorporated by reference).

### Differential Patterns of Recurrence Across Breast Cancer Molecular Subtypes

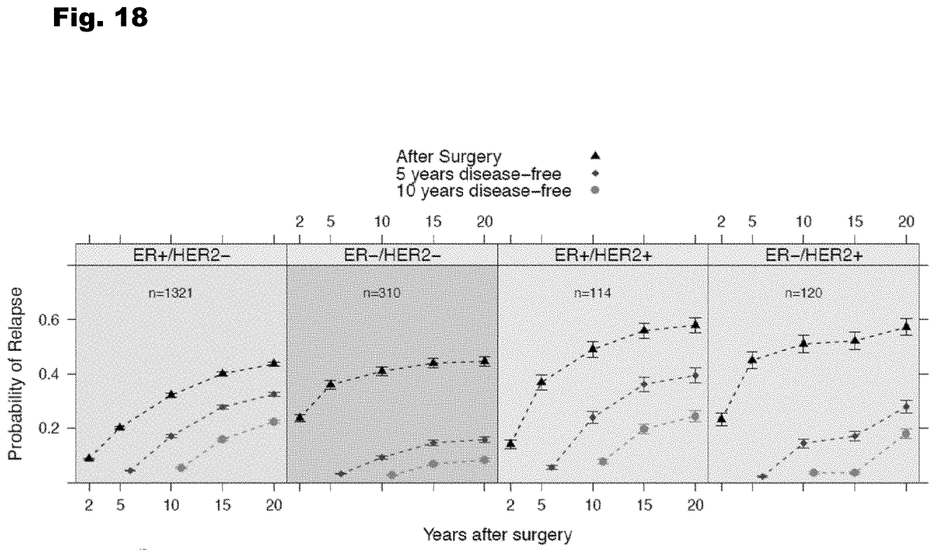

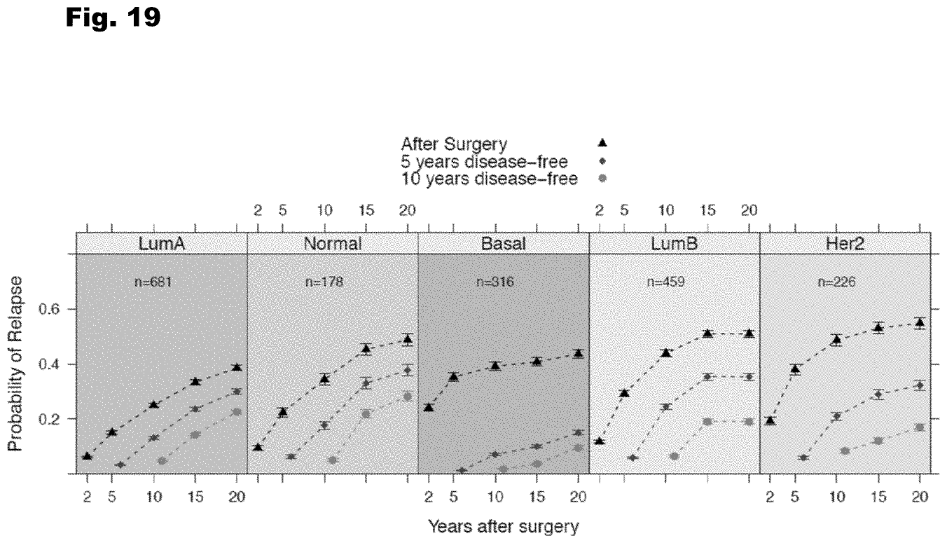

A relevant end point is the probability of experiencing a LR or DR, computed as the average probabilities of relapse among all patients. In general, the risk of LR remains relatively small, while the risk of DR changes through the course of the disease, as evident in the IntClust groups (FIG. 4), as well as the clinical (FIG. 18) and PAM50 (Fig. 19) subgroups. These comparisons further illuminate the elevated risk of LR and DR after 5 years for IntClust4ER− patients relative to IntClust10 patients. Collectively, these data indicate that amongst triple negative patients, those belonging to IntClust10 and who are relapse-free after 5 years have negligible risk of relapse, whereas the PAM50 Basal subtype and ER−/HER2− subgroups are less discriminatory.

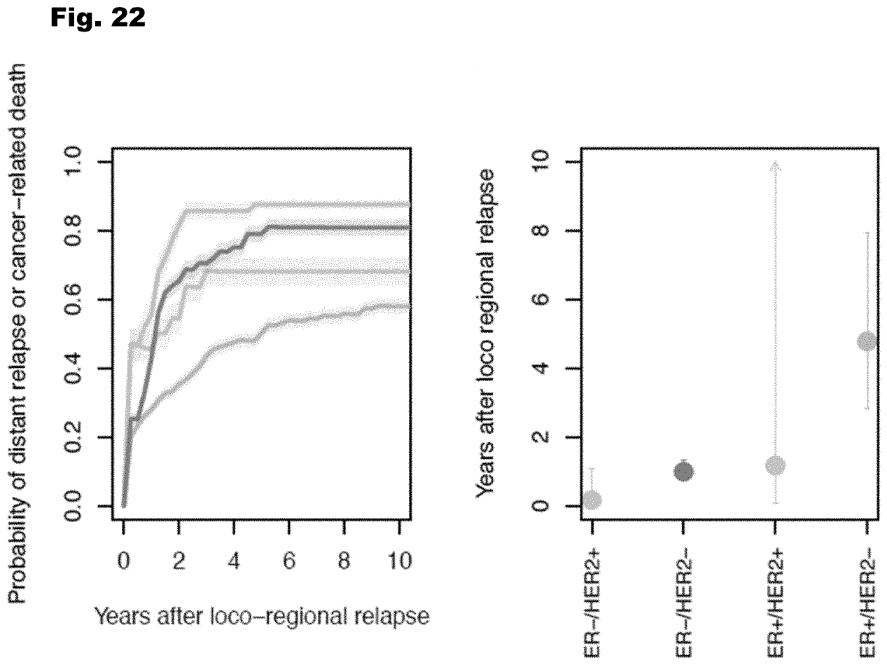

Comparisons of the probability of LR or DR also reveal dramatic differences in relapse trajectories amongst the ER+ patients with IntClust3, IntClust7, IntClust8, and IntClust4ER+ corresponding to better prognosis subgroups while IntClust1, IntClust2, IntClust6, and IntClust9 correspond to late-recurring poor prognosis patients (FIGS. 18 and 22). These four subgroups account for 26% of all ER+ cases and are at particularly high-risk of late relapse after surgery with mean probabilities of DR ranging from 0.42 to 0.55 up to 20 years after surgery. The trends are similar when restricted to ER+/HER2− cases. These high-risk ER+ subgroups thus define a sizeable minority of women who may benefit from extended monitoring and treatment given the chronic nature of their disease.

Importantly, each of the four high-risk of recurrence subgroups are each enriched for characteristic genomic copy number alterations spanning putative driver genes, corresponding to potential biomarkers (FIGS. 3A and 3B). For example, IntClust2 tumors are defined by amplification of chromosome 11q13 spanning multiple putative oncogenes, including FGF3, CCND1, EMSY, PAK1, and RSF1. IntClust2 accounts for 4.5% of ER+ cases, 96% of which have RSF1 amplification, compared to 0-22% of other subgroups. IntClust6 tumors are characterized by focal amplification of 8p12 centered at FGFR1 and ZNF703 (100% of IntClust6 cases vs. 2-21% of others) and accounts for 5.5% of ER+ tumors. IntClust1 accounts for 8% of ER+ tumors and exhibits amplification of chromosome 17q23 spanning the mTOR effector, RPS6KB1 (S6K1), which is gained or amplified in 96% and 70% of cases, respectively, whereas amplification occurs in 0-25% of other groups. IntClust9 accounts for another 8% of ER+ cases, and is characterized by amplification of chromosome 8q24 spanning the MYC oncogene with amplification occurring in 89% of IntClust9 tumors (3-42% of other groups). Collectively, these findings highlight late-recurring ER+ patient subgroups and accompanying genomic biomarkers that can be used to stratify patients and determine appropriate therapeutic strategies.

### Identification of Molecularly Defined Late-Recurring Patient Subtypes

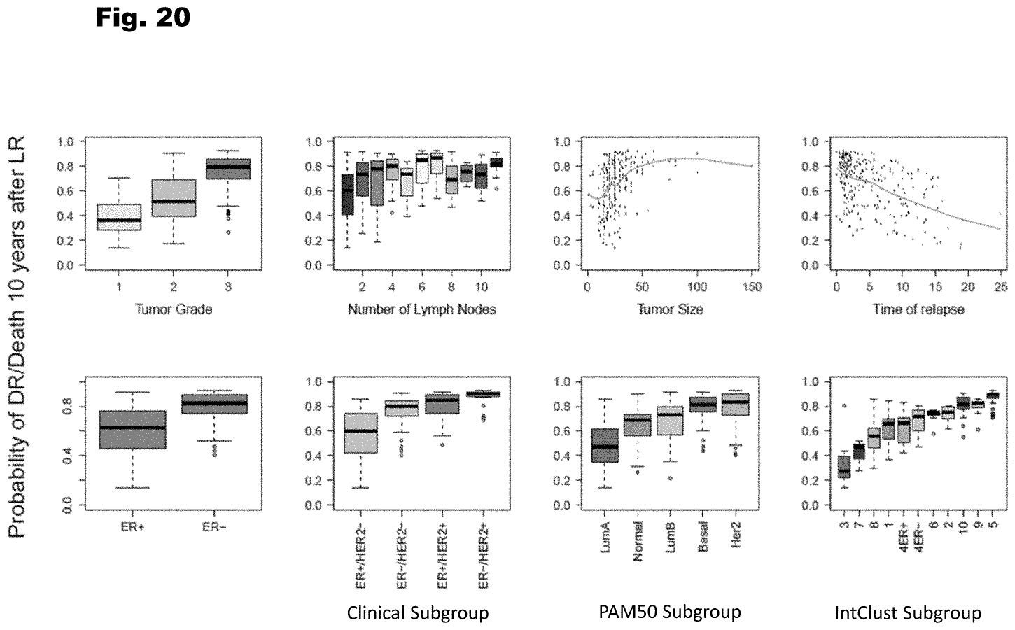

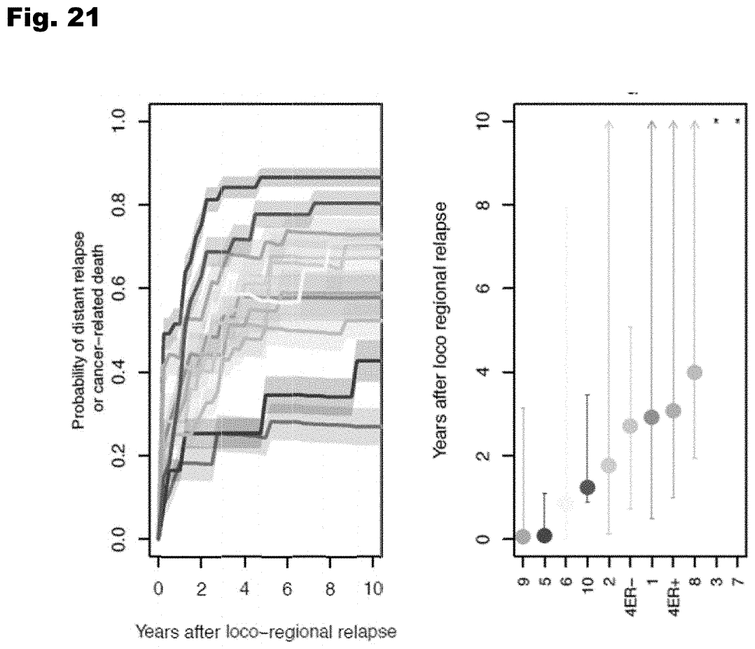

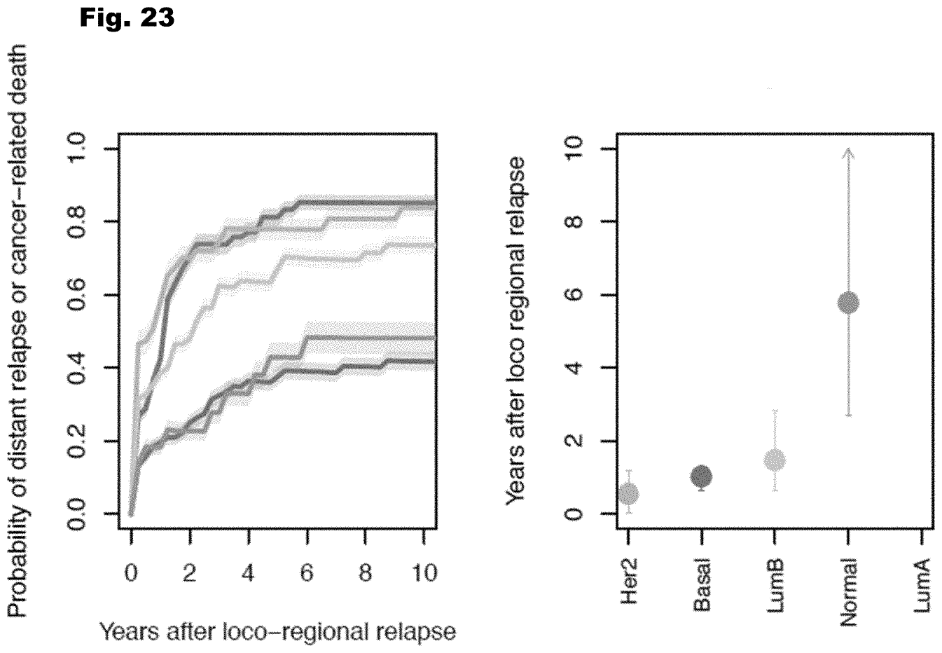

The trajectory of patient outcomes was further evaluated by comparing the average probability of progressing to DR or death for patients that had a LR (FIG. 20), which is further detailed by stratifying into the IntClust subgroups (FIG. 21), clinical classification subgroups, (FIG. 22) and PAM50 subgroups (FIG. 23). According to molecular subtype and pathological features of the primary tumor at diagnosis, the risk of DR following LR varied significantly. For example, across the IntClust subgroups, differences in risk exceed 0.6 at 10 years and this separation was more extreme than for the PAM50 subgroups. Similarly, the median time to progression varied by more than 5 years across the IntClust and PAM50 subgroups.

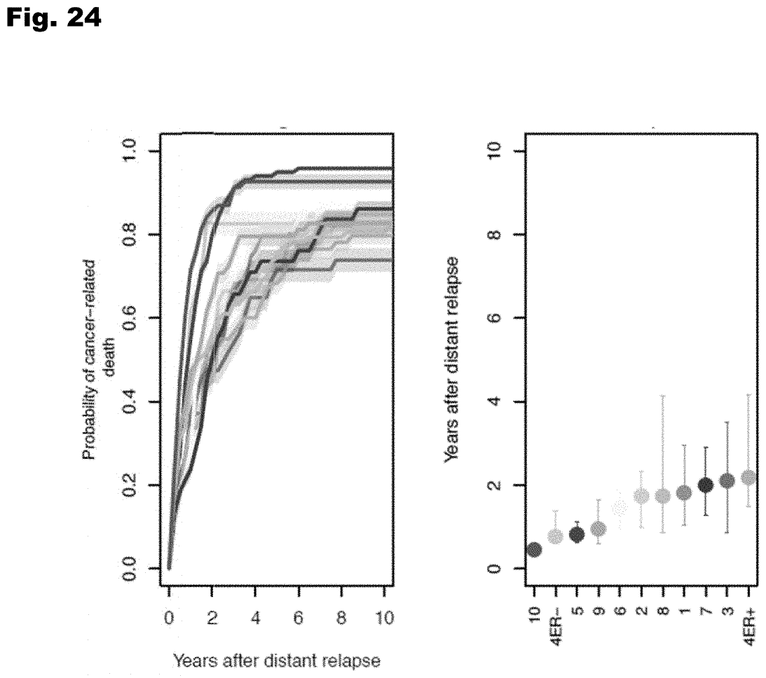

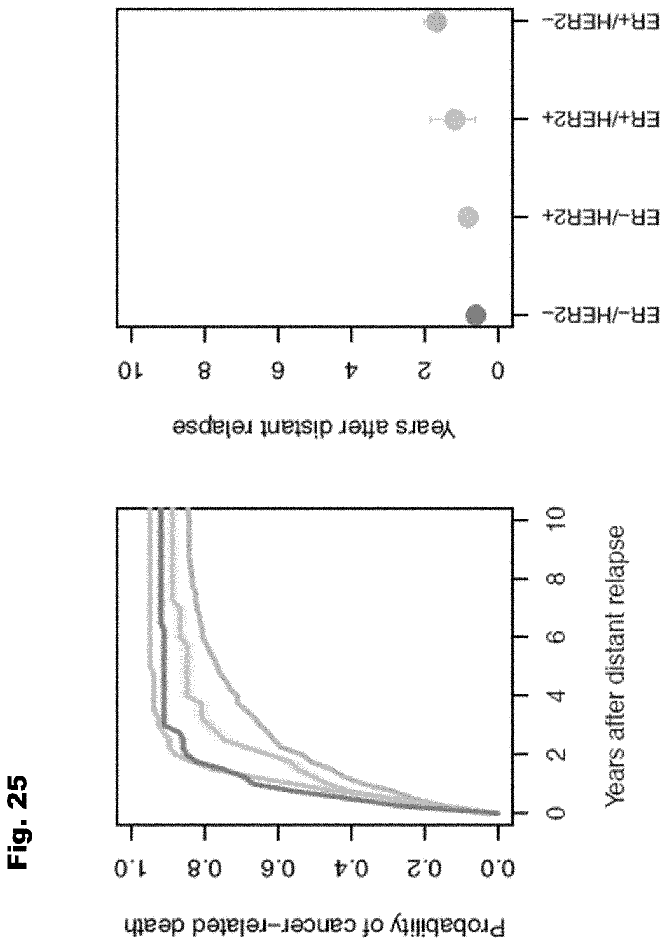

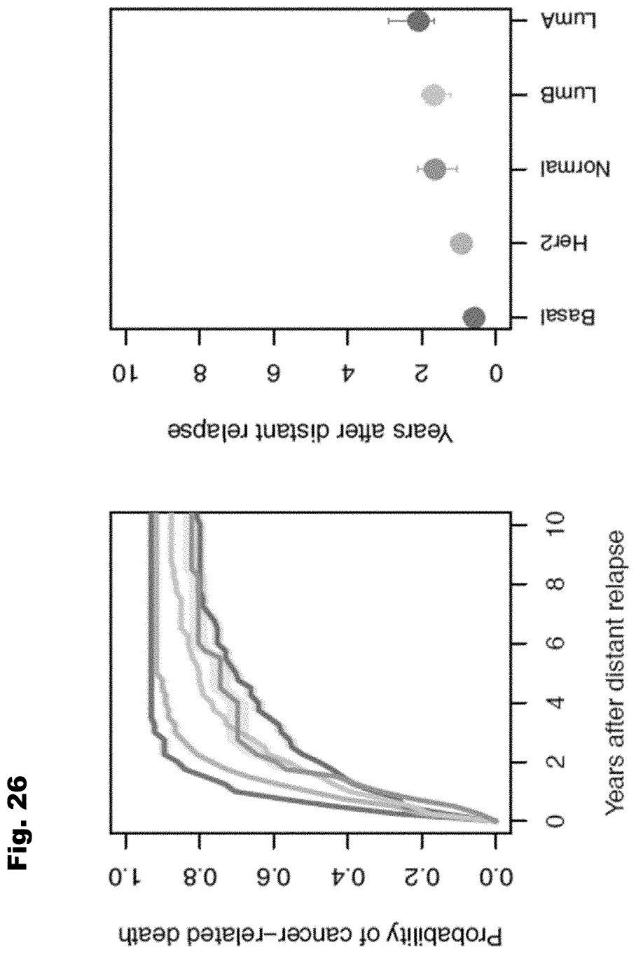

The average probability of progressing to death after DR was also evaluated and detailed by stratifying into the IntClust subgroups (FIG. 24), clinical classification subgroups, (FIG. 25) and PAM50 subgroups (FIG. 26). While the prognosis was poor for all subtypes, there were notable differences in the median time to death. These data suggest that both the pathological and molecular subtypes are still prognostic after distant relapse, as detailed further below.

### Clinical Prognostic Value of Integrative Subtyping

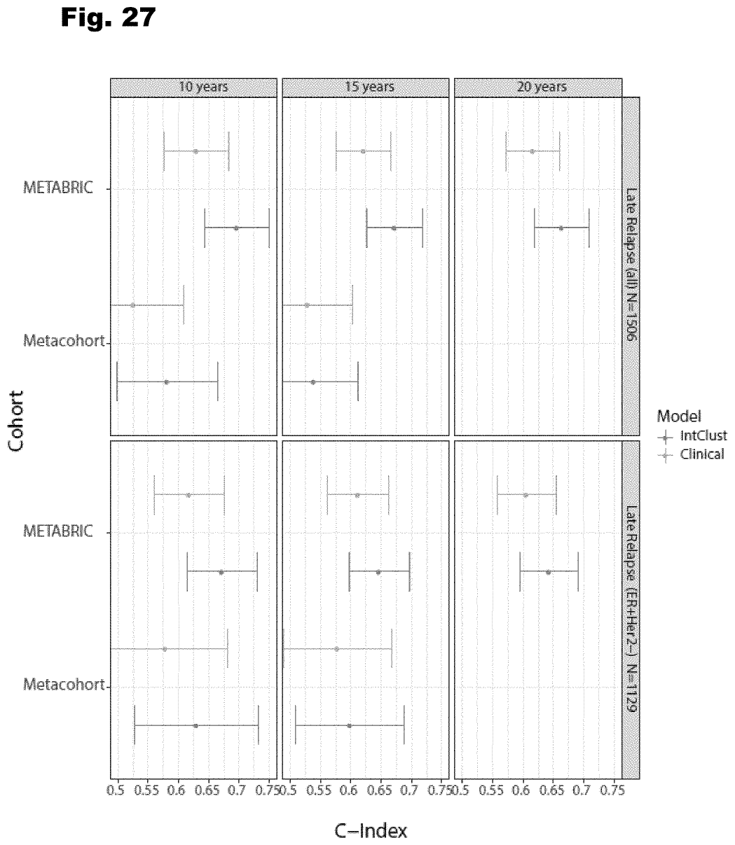

It was next assessed whether IntClust membership provided information about a patient's risk of late distant relapse above and beyond what could be inferred optimally from standard clinical information. As has been shown in other cohorts, clinical variables defined at diagnosis continued to dictate distant relapse outcomes even after a long disease-free interval. It was found that the IHC model, that included clinical variables (age, tumor size, grade, number of positive lymph nodes, time since surgery) combined with IHC subtype provided substantial information about the probability of distant relapse in patients who were relapse-free at 5 years: C-index of 0.63 (CI 0.58-0.68) at 10 years, 0.62 (CI 0.58-0.67) at 15 years, and 0.61 (CI 0.57-0.66) at 20 years. However, including the integrative subtypes significantly improved its predictive value: C-index of 0.70 (CI 0.64-0.75; improvement over the clinical model P=0.00011) at 10 years, 0.67 (CI 0.63-0.72, P=0.0016) at 15 years, and 0.66 (CI 0.62-0.71, P=0.0017) at 20 years. In other words, information about the dynamics of late relapse provided by integrative subtype could not be inferred from standard clinical variables, including IHC subtype. These trends were recapitulated in an external validation cohort despite the shorter follow-up times (prohibiting analyses at 20 years) and smaller sample size. Moreover, similar patterns were seen in the subset of patients whose tumors were ER-positive/HER2-negative (FIGS. 27-29), a group in which late relapse and strategies to target this, such as extended endocrine therapy.

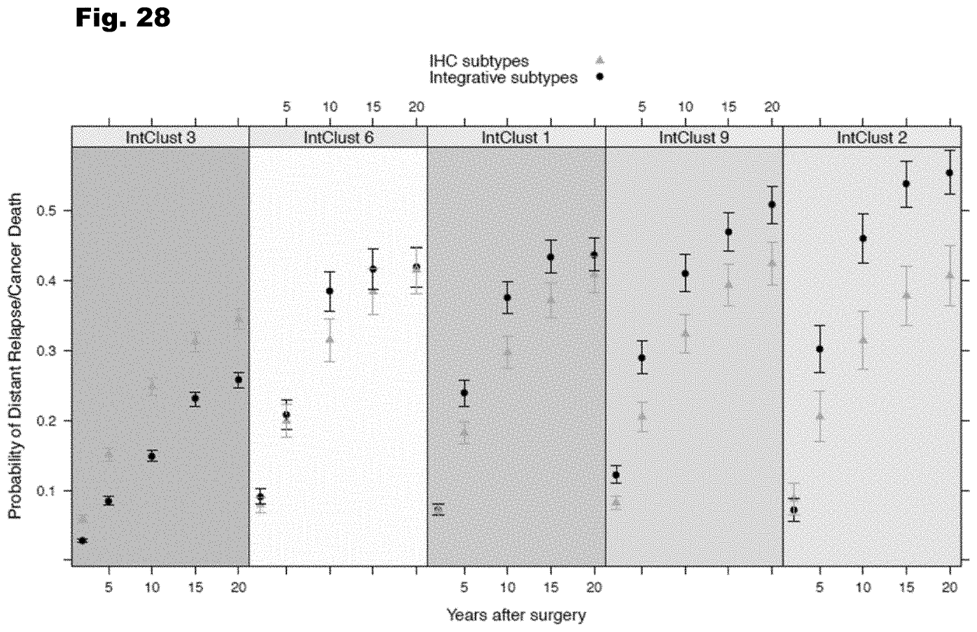

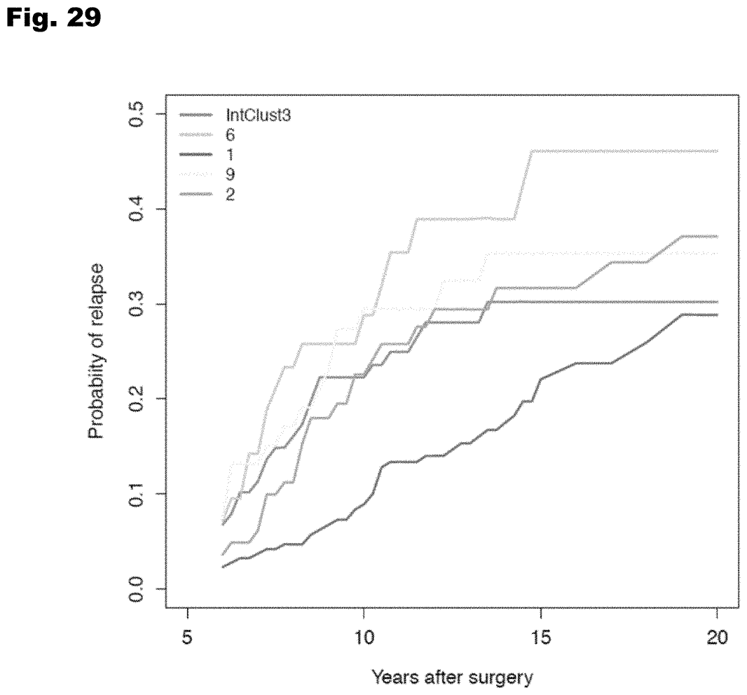

The appreciable risk of relapse associated with ER+/Her2− patients in each of these four subgroups following surgery (relative to IntClust3) varies over time and is not captured by the standard clinical model (FIG. 28). Moreover, the probabilities of DR or breast cancer death amongst individual ER+/Her2− patients who were relapse free at 5 years post diagnosis, varies considerably amongst each the four late relapsing IntClust subgroups (FIG. 29), further highlighting the importance of individualized monitoring strategies.

### Goodness of Fit Testing

Goodness of fit tests were performed for all models. Proportional hazards assumption was tested using the Schoenfeld Residuals vs. time using the survival function cox.zph( ). None of the models showed covariates that violated the assumption, except the model for sites of metastasis (ER+), where the number of metastases and “other metastasis” were significant and the model for sites of metastasis (ER−) where grade and the number of metastases were significant. Visual inspection of the plots showed that the trend was roughly flat and thus the violation was not critical. In the model that includes ER, as previously shown ER violates the proportional hazard assumption. However, this model was only used to test differences in the hazard ratios of the other covariates according to ER.

### Comparison of Probabilities of Relapse in ER+ High Risk Integrative Clusters

To test the model that stratifies risk based on the Integrative Clusters predicts different probabilities of relapse amongst the ER+ high-risk groups. The probability of having a distant relapse was computed when the patient is disease-free after surgery (defined as the probability of having distant relapse, no matter what happened next) and the probability of distant relapse/cancer death following a loco-regional relapse for ER+/HER2− patients in IntClust 1, 2, 6 and 9. A linear model with IntClust membership as an independent variable was fitted and Tukey's post hoc tests for pairwise comparisons was performed.

### Example 2: Models for Risk Stratification of Breast Cancer

A number of statistical models can be used to stratify risk of breast cancers. In many embodiments, risk stratification incorporates molecular classification and/or predictors derived from a molecular classifier (e.g., IntClust classification) as features. Molecular features can be based on gene expression and/or copy number levels, as well as DNA methylation or chromatin accessibility which reflect transcriptional levels/states.

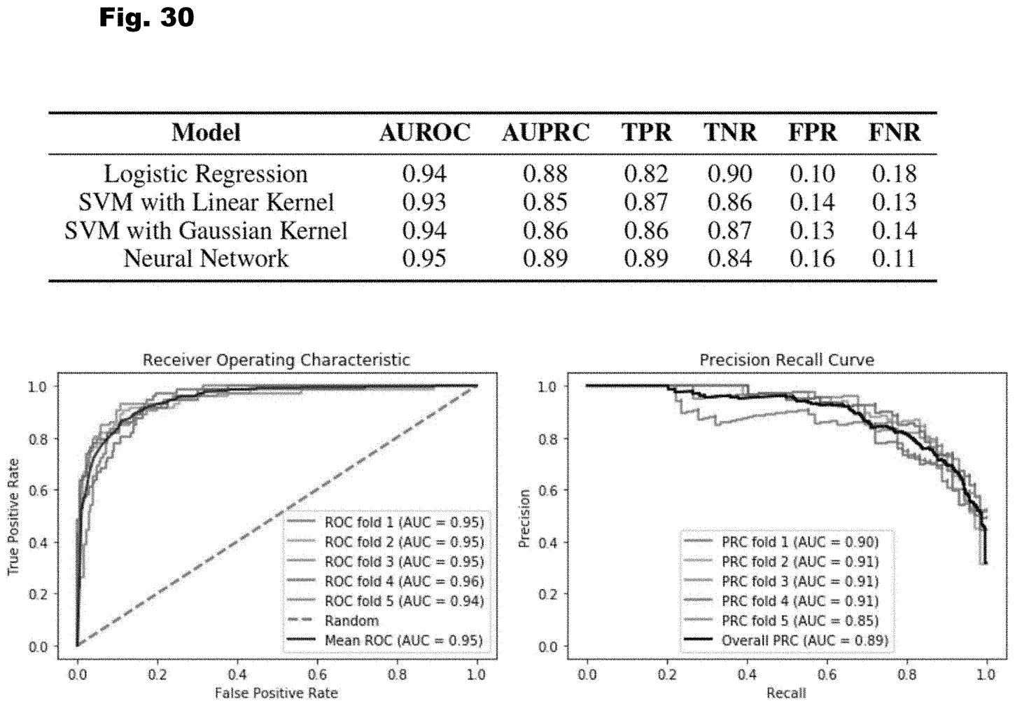

In an assessment of model performance for determining risk stratification from genome-wide copy number data, the following types of models were built and tested: logistic regression, SVM with linear kernel, SVM with Gaussian kernel, and neural network (FIG. 30).

To perform the analysis, genomic copy number from a SNP6 array consisting of 1,191,855 segments spanning the entire genome was utilized. Each segment denoted the average copy number in that region. In order to both reduce the dimensionality and obtain useful features, the CNRegions function from the iClusterPlus R package were used to merge adjacent regions and obtain a final set of 4794 consistent copy number regions for each sample (of the 1285 patients in the dataset), with adjusted mean copy number values for each region. These were used as features, alongside the clinical covariates such as age at diagnosis, tumor grade, tumor size, and number of tumor-positive lymph nodes in machine learning methods to predict integrative subtype or binary high [IC 1, 2, 6, 9] versus low [IC 3, 4, 7, 8] risk of relapse labels. The performance of various models including logistic regression, support vector machines with a linear kernel, support vector machines with a gaussian kernel, and neural networks were evaluated to determine their ability to accurately predict integrative subtype risk labels from genome-wide copy number data (FIG. 30). While multiple models performed well, the neural network has the strongest performance among the different models, with both the highest AUROC and the highest AUPRC.

### Example 3: Predicting Integrative Subtype and Risk Labels from Targeted Panel Sequencing

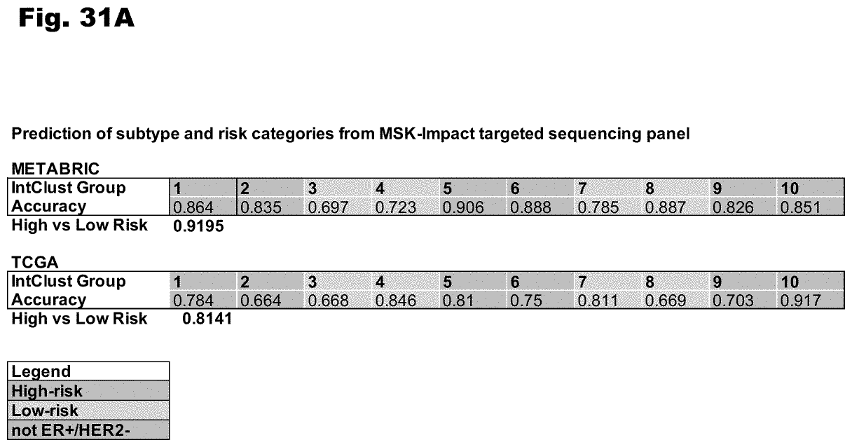

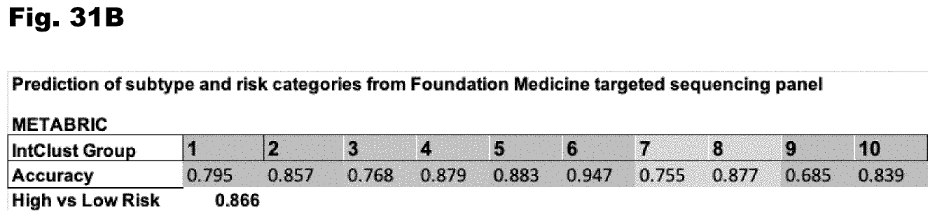

Targeted panel sequencing data (such as from MSK-Impact, Foundation Medicine or STAMP) can be utilized to predict integrative subtype and the performance of such methods can be evaluated using cohorts with genome-wide copy number (and expression data). In particular, the METABRIC and TCGA cohorts had been utilized previously for integrative subtype assignments based on the IntClust classifier (based on both gene expression and genomic copy number data). Genes in the IntClust classifier that overlap with the panel of interest were used to create a matrix consisting of Genes x Samples, where for each tumor, segmented copy number values based on the circular binary segmentation (CBS) algorithm are used. Alternatively, all genes on the panel can be utilized, again resulting in a matrix consisting of Genes x Samples, for each tumor, where for each tumor, segmented copy number values based on the circular binary segmentation (CBS) algorithm are used. The PAM algorithm from the pamR package was used to train the classifier in the METABRIC (or TCGA training set) using cross-validation to select the proper shrinkage parameter (i.e., optimizing F1). Breast tumors were classified into the Integrative Subtypes and the class labels for the training and withheld test set compared with the well validated IC10 assignments (based on genomic copy number and gene expression data). Measures of performance, including balanced accuracy were evaluated for assignments to each of the 10 groups and for the binary risk categories amongst ER+/Her2− tumors, namely high risk (IntClust subgroups 1, 2, 6, 9) vs lower risk (IntClust subgroups 3, 4, 7, 8) or relapse (FIGS. 31A and 31B) and demonstrate the robust classification of integrative subtype from targeted (panel) sequencing data which is available through several companion diagnostic assays.

An alternative approach for predicting integrative subtype from panel sequencing data involves step-wise binning. In this approach, copy number estimates for METABRIC generated using ASCAT were used (for more on ASCAT, see P. Van Loo, et al., Proc Natl Acad Sci U S A. 2010;107(39):16910-16915, the disclosure of which is incorporated herein by reference). These copy number calls were subsetted to genes within the FoundationOne panel. Fraction of genome altered (FGA) was computed for the genes and the METABRIC data was filtered to include samples with FGA>0. This resulted in 510 samples to train a classifier. The copy number estimates was then transformed using a binning approach to avoid over-fitting to specific copy number profiles. For this, the following bins −0-6, 6-10, 10-14, 14-20, 20-60 and >60 were used. Additionally, arm level copy number estimates for the chromosomal arms relevant to the high risk subgroups were incorporated (i.e. 8p11, 8q24, 11q13 and 17q23).

IntClust1, IntClust2, IntClust4, IntClust6, IntClust8 and IntClust9 were used for training, maximizing the accuracy for the four high risk categories, namely IntClust1, IntClust2, IntClust6 and IntClust9. The model uses a voting based approach incorporating elastic net regression, random forest and gradient boosted tree to infer the IntClust type for a given sample. While the overall accuracy was 69% across all subtypes, reasonably high test accuracy for the high risk groups was achieved as shown below.

| Group | Precision | Recall | F-score |
|---|---|---|---|
| IntClust1 | 76% | 87% | 81% |
| IntClust2 | 100% | 83% | 91% |
| IntClust6 | 87% | 87% | 87% |
| IntClust9 | 75% | 94% | 83% |

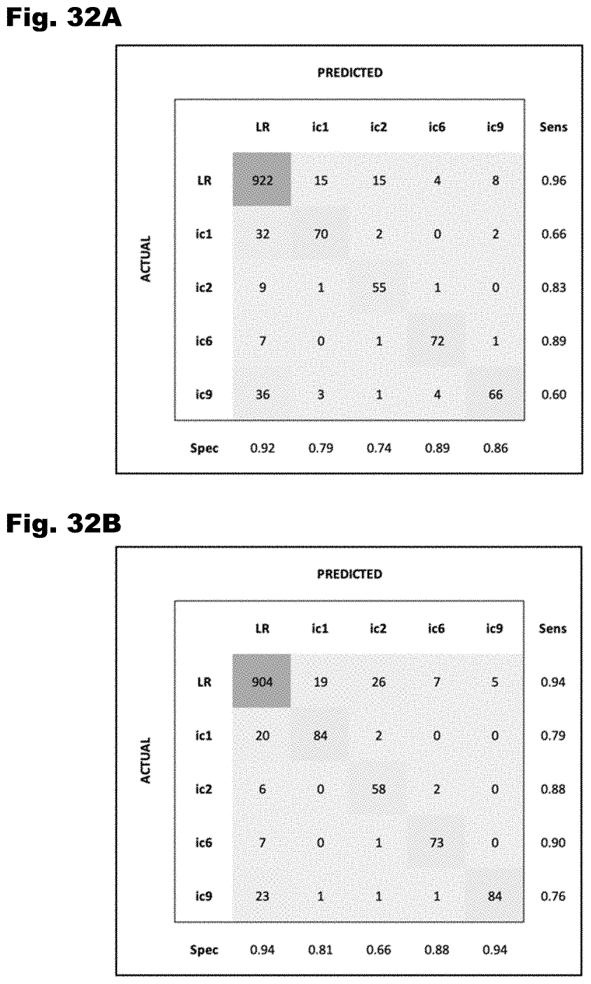

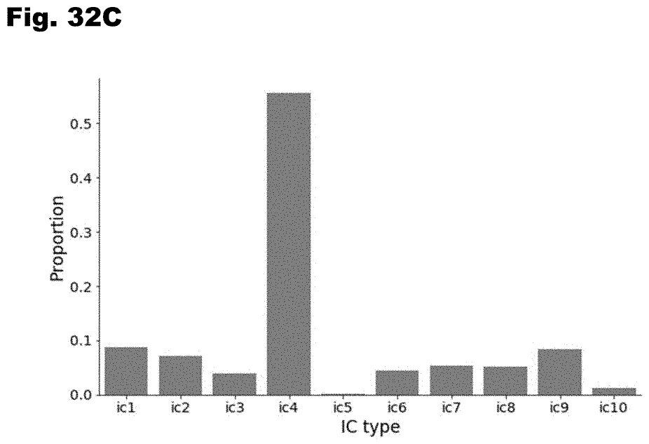

The overall train+test accuracy for all METABRIC samples is shown in FIG. 32A. For the Foundation Medicine data, copy number estimates from the clinical reports provided by Foundation Medicine Inc. were used. These include amplifications of 6 copies or higher. Starting with the reported CN calls, the binning was performed as described above and computed arm level copy number estimates for the chromosomal arms of interest. This was then used as input to the classifier above to make predictions on the Foundation Medicine data.

The MSK cohort comprises of 1918 samples from 1756 patients, of which 1345 ER-positive and HER2-negative samples were analyzed. In order to identify integrated subtypes from the MSK data, a classifier-based approach was developed using the genes present in the MSK-IMPACT panel. For this, the original METABRIC cohort was used to first identify the 10 integrative subtypes. Among the METABRIC samples, 1363 were ER-positive HER2-negative and these were the samples used to develop the IMPACT-IC classifier.

Copy number estimates for METABRIC generated using ASCAT were used (P. Van Loo et al., cited supra). These copy number calls were subsetted to the genes of the MSK-IMPACT panel. Fraction of genome altered (FGA) was computed for the genes and the METABRIC data was filtered to include samples with FGA>0. This resulted in 611 samples to train the classifier. The copy number estimates were then transformed using a binning approach to avoid over-fitting to specific copy number profiles. For this, the following bins −0-6, 6-9, 9-12, 12-15, 15-20, 20-60 and >60 were used. For genes that are most important for IntClust1 prediction (as determined from feature importance values from elastic net regression), the first two bins were lowered to 0-4, 4-9. Additionally, arm level copy number estimates were incorporated for the chromosomal arms relevant to the high risk subgroups (i.e. 8p11, 8q24, 11q13 and 17q23).

Although all 10 IntClust subtypes were used for training, maximizing accuracy for the four high risk categories, namely IntClust1, IntClust2, IntClust6 and IntClust9. The model uses a voting based approach incorporating elastic net regression, random forest and gradient boosted tree to infer the IntClust type for a given sample. While the overall accuracy was 68% across all subtypes, reasonably high test accuracy was achieved for the high risk groups as shown below.

| Group | Precision | Recall | F-score |
|---|---|---|---|
| IntClust1 | 57% | 81% | 67% |
| IntClust2 | 71% | 92% | 80% |
| IntClust6 | 83% | 94% | 88% |
| IntClust9 | 100% | 94% | 97% |

The overall train+test accuracy for all METABRIC samples is shown in FIG. 32B The precision for IntClust1 is relatively lower due to this group being characterized by low level gains of 17q23 arm as opposed to high level amplifications.

For the MSK dataset, allele specific copy number estimates were generated utilizing FACETS (see R. Shen and V. E. Seshan, Nucleic Acids Res. 2016;44(16):e131, the disclosure of which is incorporated herein by reference). FACETS results were provided by Memorial Sloan Kettering Cancer Center. An initial quality control of the copy number profiles was performed and in cases where there were multiple possible fits, the best fit was chosen based on several metrics including rate of homozygous deletions, rate of loss of heterozygosity and balanced chromosomal segments. Although the two methods used for copy number calling were different, they are both allele-specific in nature and correct for tumor purity in the copy number estimates. Starting with the FACETs calls, the binning was performed as described above and computed arm level copy number estimates for the chromosomal arms of interest. This was then used as input to the classifier above to make predictions on the MSK-IMPACT data.

There are 3 versions of the panel in use among these patients, the IM3 with 341 genes, the IM5 with 410 genes and the IM6 with 468 genes. In order to account for the difference in the content of these panels, some parameters were slightly modified to optimize performance in the versions of the panel with fewer genes.

Of the 1345 samples that were subtypes, 385 fell into high risk categories. This was not significantly different from the proportion of high risk subtypes within METABRIC (Fisher's exact p-value=0.26). The overall distribution of integrative clusters is shown in FIG. 32C. This result suggests that the classifier captures the key groupings.

Among the 1344 samples from the MSK-cohort, 728 samples were from primary tumors and the remaining 616 were from metastatic lesions. When comparing the distribution of primary and metastatic tumors, it can be seen that the proportion of high risk integrative clusters in the metastatic samples is significantly higher than that seen in the samples from primary tumors (odds ratio 1.76, fisher's exact p-value=3.98e-06), reflecting the fact that the high-risk IntClust groups indeed confer increased risk of relapse.

### Example 4: Benchmarking the Performance and Clinical Utility of Integrative Subtyping for ER+/HER− Breast Cancer

Utilization of the IntClust classification system results in better performance in predicting distance relapse than the currently marketed diagnostic tests, especially in ER+/HER2− breast cancer. In this example, Integrative subtyping is compared to Oncotype Dx (Genomic Health, Redwood City, Calif.), Prosigna (NanoString Technologies, Seattle Wash.), MammaPrint (Agendia, Irvine, Calif.), and Breast Cancer Index (BCI) (Biotheranostics, Inc., San Diego, Calif.).

Score and risk were generated for each test per their protocol and using the genefu Gene Expression Based Signatures in Breast Cancer (D.M. Gendoo, et al., http://www.pmgenomics.ca/bhklab/software/genefu). In regards to the IntClust classification, high risk is classification into IntClust subgroups 1, 2, 6 or 9 and lower risk is classification into IntClust subgroups 3, 4, 7 or 8. IntClust scores were calculated as distance to the closest high risk centroid. Prosigna's PAM50 was used to compute an RoR score and further used the subgroups to categorize risk and score: high risk is classification into LumB, lower risk is classification into LumA and score was determined by probability of LumB. For BCI, score was calculated by [0.44*(first PC prolif)+0.4972*(hoxb12/IL17RB ratio) — 0.09 (hoxb12/IL17RB ratio)\hat{ }3]*2+5; and risk is high if score was greater than 6.4 and risk is low if score was less than 5.

The METABRIC dataset was used to generate signatures from gene expression data as detailed in Curtis, et al., (2012), cited supra. Outcome associations, including late relapse, of the METABRIC cohort were also calculated as detailed in Example 1. In this example, the data was limited to ER+/HER2− samples (n=1398). Late relapse is defined as relapse that occurs after 5 years without any previous incidents of relapse after surgery (i.e., relapse free at year 5). Two outcomes were considered, distant relapse free survival and relapse free survival. Distant relapse free survival is defined as time to distance relapse. Relapse free survival is defined as time to distant relapse or disease specific death.

To perform outcome analyses, Kaplan Meier plots were generated using the survival packages (model using survfit function) and survminer (plt, using ggsurvplot function. P-values were generated using Logrank test. Hazard ratio was calculated with hazard.ratio function from survcomp package, which was used to measure the effect size of the signature. Concordance Index (C-Index) was calculated using concordance.index from survcomp package. Area under the curve was used to evaluate the prediction performance of the signatures in different time points. Uno's AUROC from AUC.uno function of survAUC package was used to calculate AUROC. To better compare the improvement in prediction with respect to clinical covariates, for each timepoint, the AUC was calculated using a Cox Proportional Hazard model using the risk or the scores along with adjusted clinical covariates. A 20×10-fold cross validation was performed to avoid overfitting in the overestimation of the AUC.

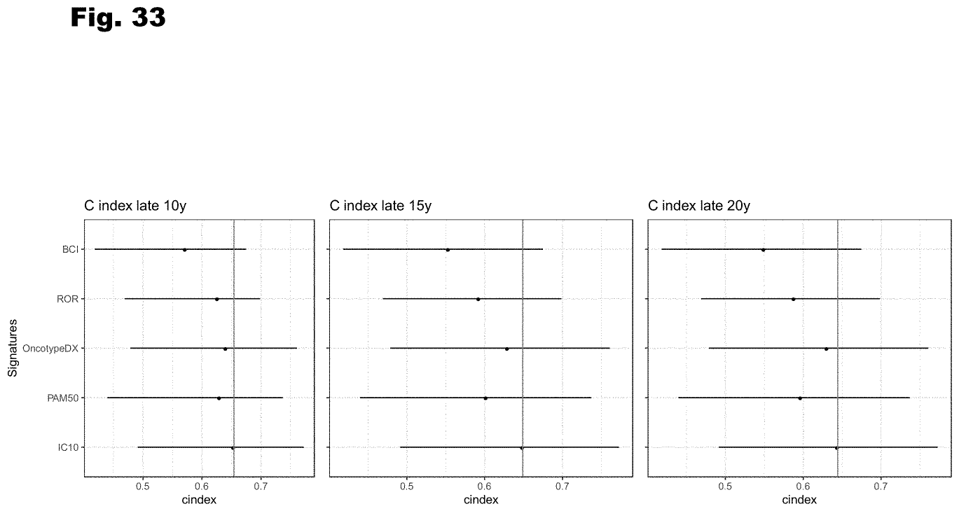

Provided in FIG. 33 are C-index scores for BCI, Prosigna's ROR, Oncotype DX, Prosigna's PAM50 and the IntClust classification (IC10). The C-index scores were calculated for the ability to predict a late relapse at 10 years, 15 years, and 20 years. As can be seen, the IntClust classification outperforms the other diagnostic tests at each timepoint.

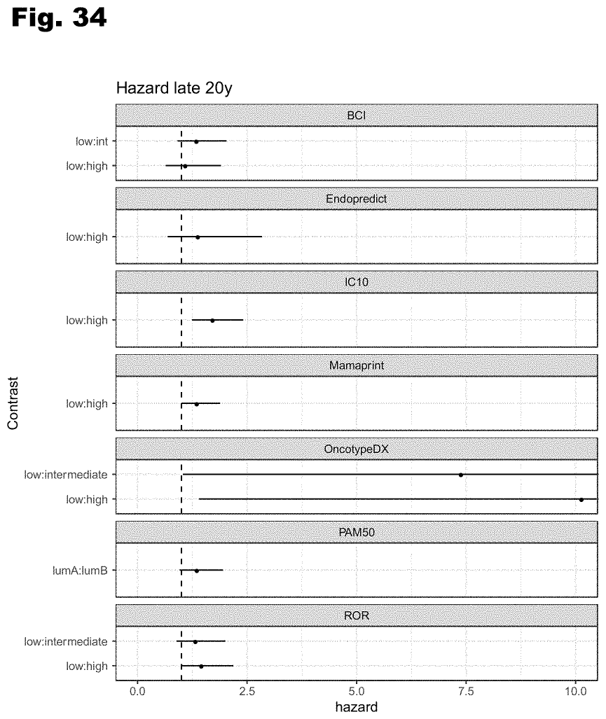

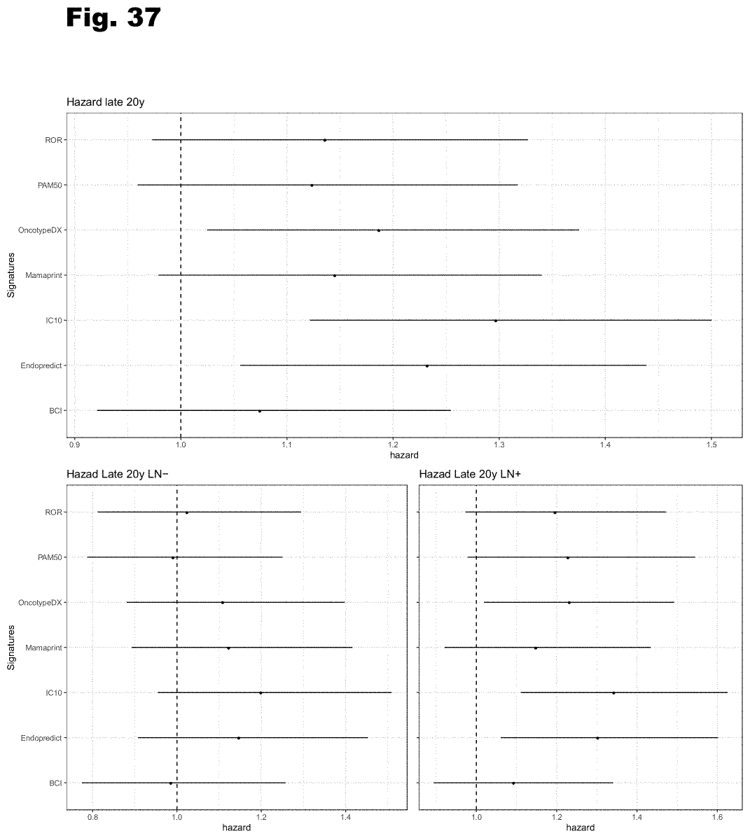

Provided in FIGS. 34 to 37 are hazard ratio (HR) plots of late distant relapse. FIG. 34 provides HR of late distant relapse amongst ER+/HER2− patients (in some cases stratified by lymph node status) who were relapse-free at 5 years for different multigene signatures and corresponding risk categories. Whereas the confidence intervals for most signatures overlap the equality line (one), indicating that they are not significantly associated with differential risk of late distant relapse, high versus lower risk IntClust stratification (IC10) exhibits a significantly elevated HR. Further, the error bars for Oncotype Dx are particularly wide. This is due to the fact that the Oncotype Dx resulting low risk group is extremely low risk and includes very few patients. Many more patients are stratified into the intermediate risk group (for which treatment issues are less clear). Indeed, the use of arbitrary thresholds for binning individuals into risk categories when comparing the hazard ratios for different multigene signatures can create artifacts, complicating the interpretation of the results (FIG. 34-36). For this reason, it is preferable to compare scores for each signature as shown in FIG. 37. This effect is also mitigated when comparing C-indices (FIG. 33).

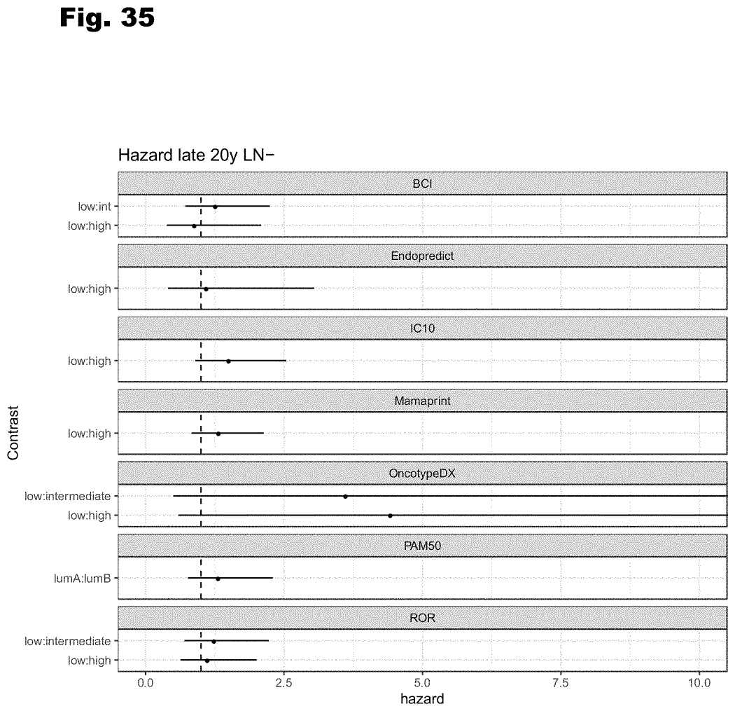

FIG. 35 provides HR of late distant relapse amongst ER+/HER2−, lymph node negative patients who were relapse-free at 5 years for different multigene signatures and corresponding risk categories. High versus lower risk IntClust stratification (IC10) exhibits the highest HR amongst all signatures.

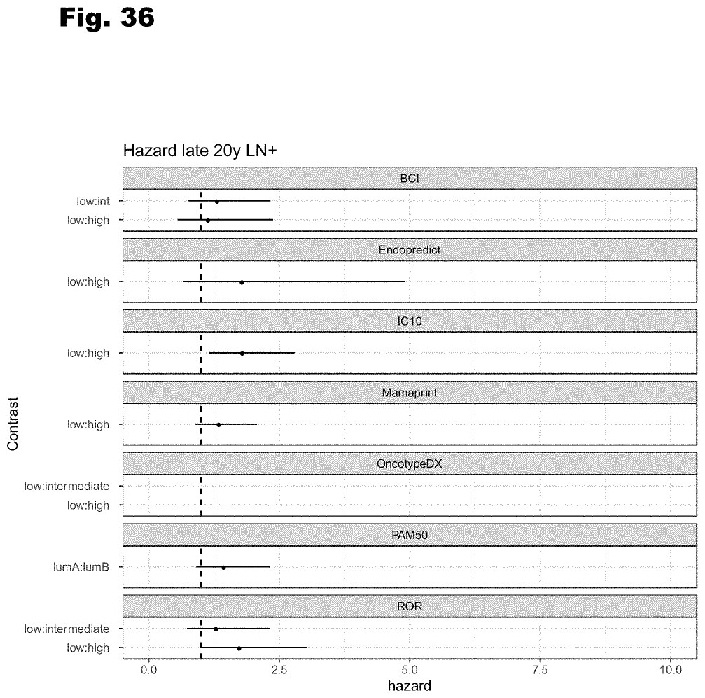

FIG. 36 provides HR of late distant relapse amongst ER+/HER2−, lymph node positive patients who were relapse-free at 5 years for different multigene signatures and corresponding risk categories. Whereas the confidence intervals for most signatures overlap the equality line (one), indicating that they are not significantly associated with differential risk of late distant relapse, high versus lower risk IntClust stratification (IC10) exhibits a significantly elevated HR. Note that Oncotype Dx is not shown due to the low number of events in the low risk group.

FIG. 37 provides HR of late distant relapse amongst ER+/HER2− patients who were relapse-free at 5 years for different multi-gene signatures. Here a score was computed to facilitate comparisons between high versus lower risk categories for each multigene signature. Whereas the confidence intervals for most signatures overlap the equality line (one), indicating that they are not significantly associated with differential risk of late distant relapse, high versus lower risk IntClust stratification (IC10) exhibits a significantly elevated HR, as particularly evident in all cases and lymph node positive cases (right panel).

### Example 5: Combining Integrative Subtyping with Other Diagnostic Tests

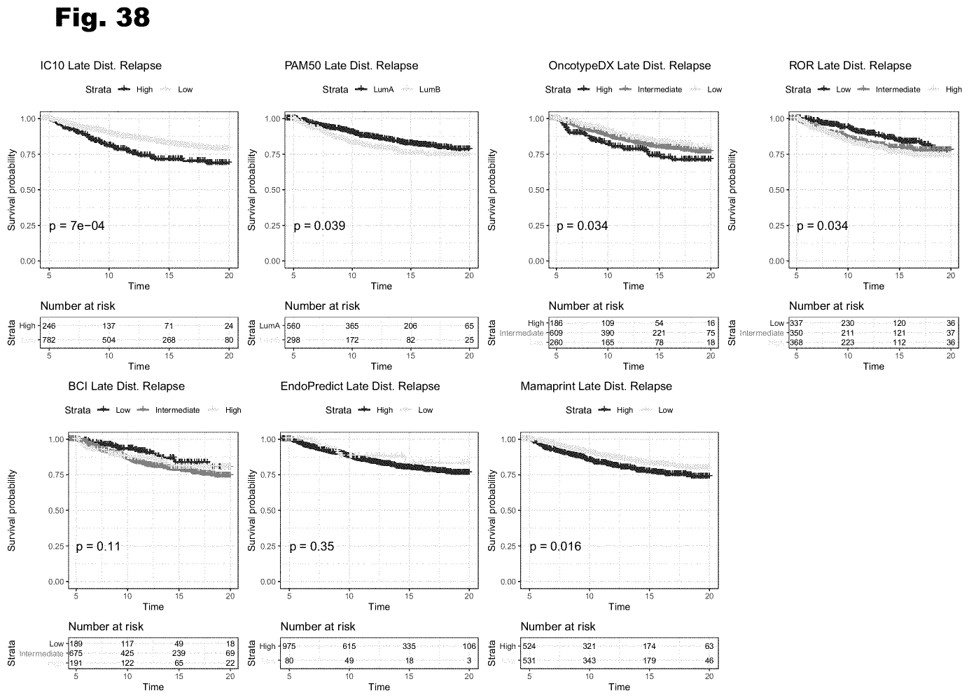

Provided in FIG. 38 are survival probability curves for late distant relapse of a number of diagnostic tests, including IntClust stratification (IC10), OncotypeDX, PAM50, ROR, BCI, EndoPredict and MammaPrint. To obtain these curves, the METABRIC data set that included late relapse data of a cohort of ER+/HER2− patients was utilized to predict risk by each diagnostic test. The patients within METABRIC cohort were assigned to the risk group as determined by each diagnostic test, according to their methods. The late distant relapse survival probability (i.e., relapse beyond 5 years of diagnosis) of each risk group was plotted.

The signatures for each diagnostic test were computed as follows:
IC10: IC10 assignments from Curtis et al. 2012; Rueda et al. 2019 (cited supra) were used. Samples assigned to IntClust subgroups 1, 2, 6 and 9 were considered high risk, whilst samples assigned to IntClust subgroups 3, 4, 7 and 8 were considered lower risk. Samples assigned to IntClust subgroups 10 and 5 were discarded when predicting risk of relapse in ER+/HER2− disease. The IC10 score is calculated by measuring the maximum posterior probability of belonging to the high risk groups where posterior probabilities are calculated from the predict function of the pamR package.
PAM50: The genefu package molecular.subtyping function was used to calculate the PAM50 assignments for the METABRIC dataset. Luminal B/LumB were assigned to the high risk group, and Luminal A/LumA and Normal like to the lower risk group. The pam50 score is defined as the posterior probability of LumB assignment.
OncotypeDX: A modified version of the oncotypedx function in the genefu package was used to call OncotypeDX score and risks and leveraged an external cohort with actual oncotypeDX values and expression data available to recalibrate the model. Values higher than 31 were considered high risk, lower than 18 low risk, and those in between are intermediate risk.
Prosigna ROR (ROR): The genefu package rorS function was used to compute the Prosigna (PAM50) risk of relapse (ROR) score, which is scaled from 1:100. Values lower than 29 were consider low risk, those higher than 52 were considered high risk, and the remainder intermediate risk.
BCI: The BCI score was calculated by combining a proliferation signature with the ratio between HOXB13 and IL17RB (hiratio) such that BCI=0.4431*prolif+0.4972*hiratio−0.09hiratio\hat{ }3). The proliferation signature is the first principal component of the expression of the following genes: BUB1B, CENPA, NEK2, RACGAP1 and RRM2. BCI was scaled by multiplying by 2 and adding 5. Values higher than 6.4 were considered high risk, those lower than 5 were considered low risk, and the remainder intermediate risk.
Endopredict: The endopredict function in the genefu package was used to calculate the Endopredict score and risk. Values higher than 5 were considered high risk and the remainder were considered low risk.
Mammaprint: The mammaprint function in the genefu package was used to calculate the Mammaprint score and risk, where values higher than 0.3 were considered high risk, and the remainder were considered low risk.
   To standardize comparisons, all scores were scaled to mean 0, standard deviation of 1.

As can be seen in FIG. 38, integrative subtype (IC10) provides much better stratification between high and lower risk groups in terms of survival from late distant relapse. In fact, IC10 is the only signature to robustly stratify high versus lower risk of late distant relapse. In other words, utilization of an IC10 diagnostic provides a better indicator of the risk that an ER+/HER2− patient has of experiencing a relapse beyond 5 years. MammaPrint provided the second best stratification, followed by OncotypeDX and ROR, but these were far more modest than that achieved by IC10.

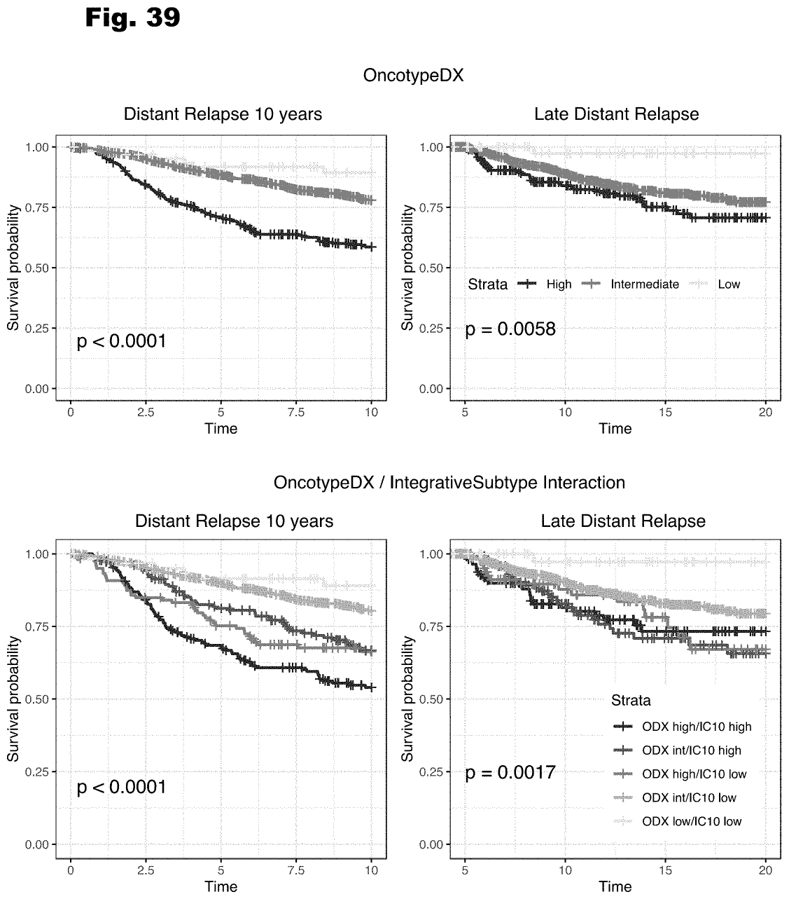

Provided in FIGS. 39 to 43 are survival probability curves for late distant relapse of a number of diagnostic tests, including OncotypeDX, PAM50, ROR, BCI, and MammaPrint, and their combination with IC10. To obtain these curves, the METABRIC data set that included late relapse data of a cohort of ER+/HER2− patients was utilized to predict risk by each diagnostic test. The patients within METABRIC cohort were assigned to the risk group as determined by each diagnostic test and in combination with integrative subtype IC10, according to their methods. The distant relapse within 10 years and late distant relapse survival probability (i.e., relapse beyond 5 years) of each risk group were plotted.

As can be seen in FIGS. 39 to 43, combining IC10 with each diagnostic test improved the stratification of patients for prediction of risk of late distant relapse. These results provide the combination of an integrated cluster system with these genetic tests improves their diagnostic ability, especially for late distant relapse.

Of particular interest in the combination of integrative cluster testing with Oncotype DX, which is a popular diagnostic test to determine treatment for ER+/HER− breast cancer. The test examines expression of 21 genes, which is used to tailor treatments, especially in individuals with early-stage ER+, HER2− breast cancer. Oncotype Dx quantifies the likelihood of distant recurrence within 10 years, providing a score that indicates a high, intermediate, or low likelihood of recurrence. It is noted that results indicating intermediate likelihood of recurrence can often present a clinical conundrum for clinicians and thus does not provide a good indication of which treatment to perform.

Combining the IC10 classification system and Oncotype DX resulted in a better stratification than Oncotype DX alone and much more clearly stratified the Oncotype DX intermediate risk group in both distant relapse within 10 years and late distant relapse (FIG. 39). The combined Oncotype DX intermediate risk and IntClust high risk group is clearly much more likely to have a relapse as compared to the combined Oncotype DX intermediate risk and IntClust lower risk group. This result indicates that combining Oncotype DX with an IntClust classification can provide better prediction of relapse risk than Oncotype DX alone, especially for the intermediate group risk group.

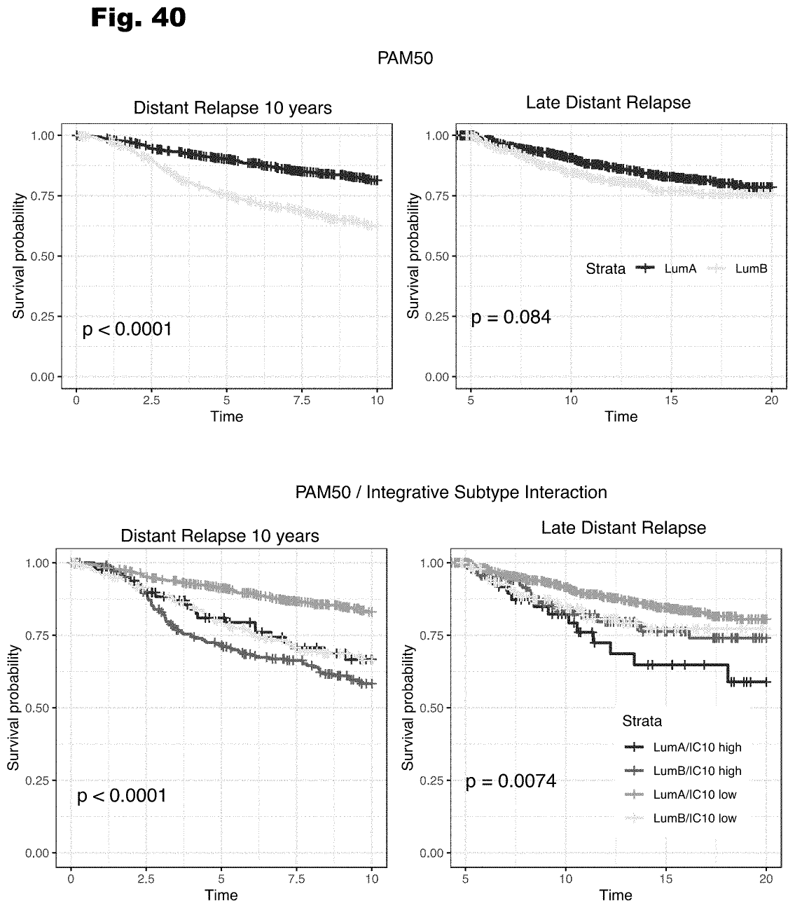

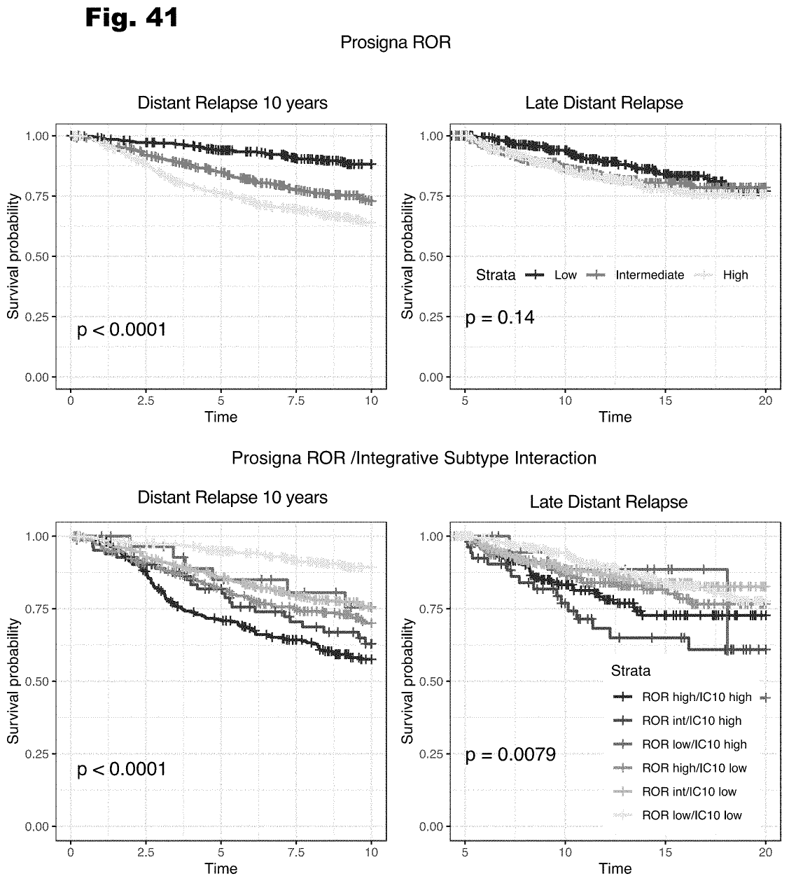

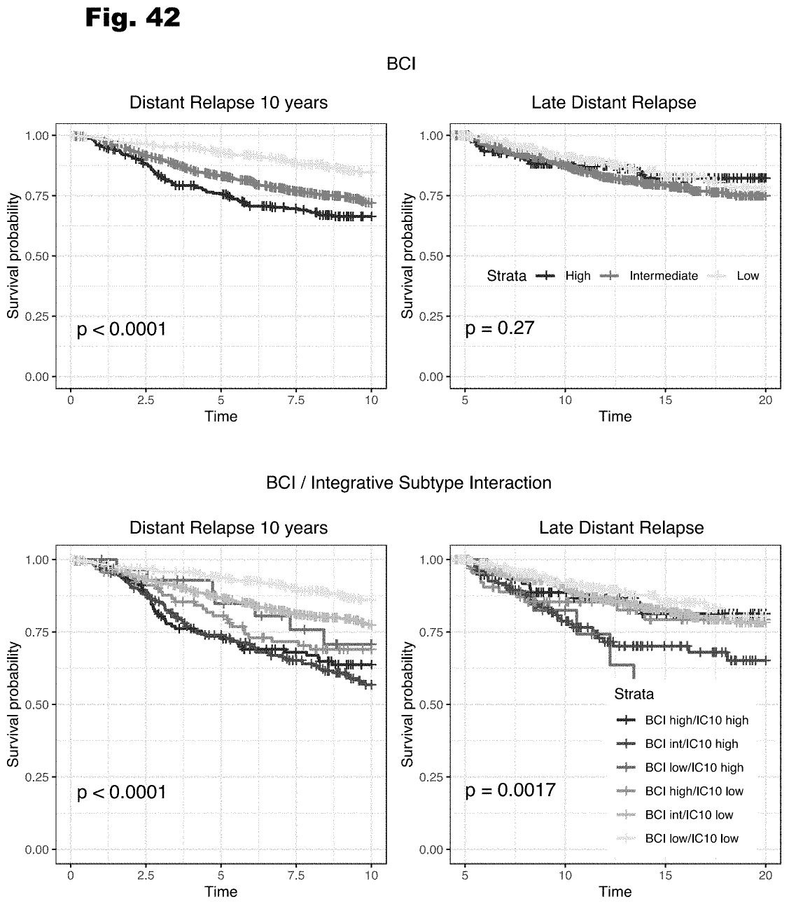

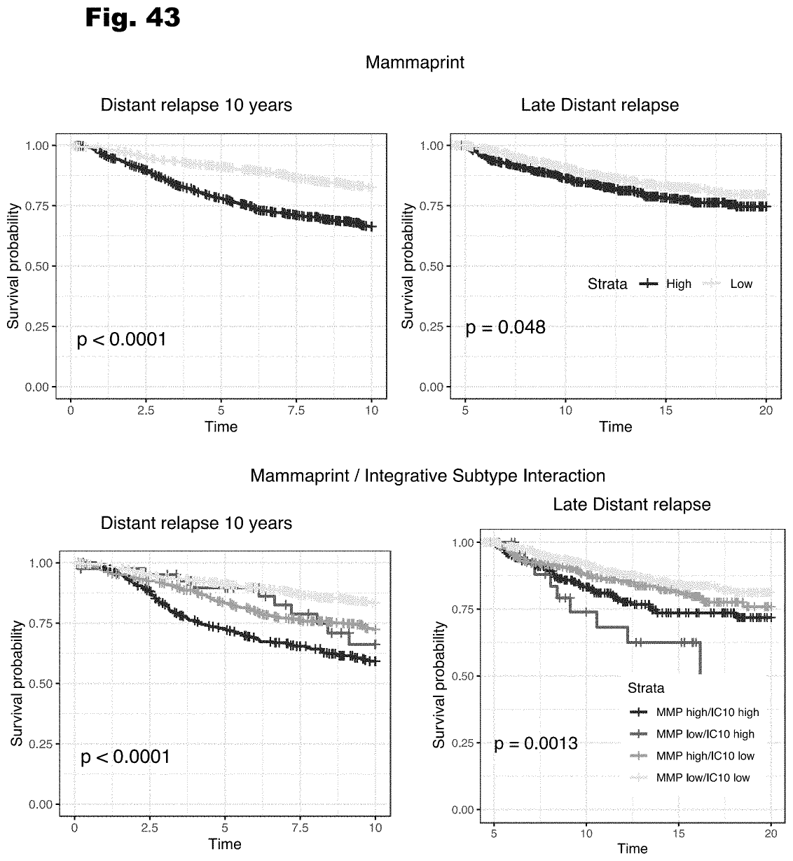

Combining the IC10 classification with PAM50 also improved stratification of the LumA and LumB groups in both distant relapse within 10 years and late distant relapse (FIG. 40). Combining the IC10 classification with ROR also improved stratification of the intermediate risk group in both distant relapse within 10 years and late distant relapse (FIG. 41). Combining the IC10 classification with BCI also improved stratification of the intermediate risk group in both distant relapse within 10 years and late distant relapse (FIG. 42). Combining the IC10 classification with MammaPrint also improved stratification of the lower risk group beyond 5 years and especially for late distant relapse (FIG. 43).

### Example 6: Treatment Results on Particular Molecular Subgroups

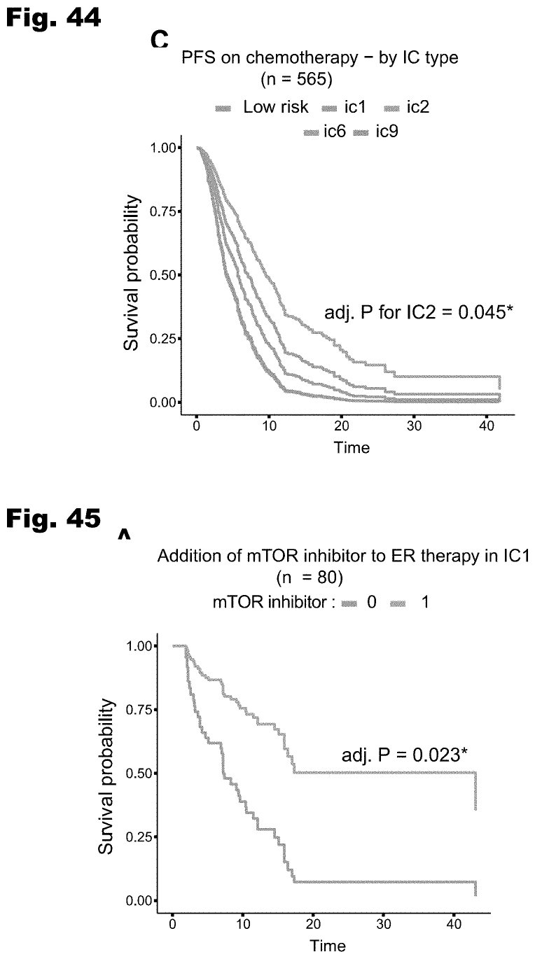

The ability of chemotherapy, targeted therapies, and endocrine therapies on patients within particular molecular subgroups was examined in a prospective cohort of 812 patients with metastatic ER-positive breast cancer. Provided in FIG. 44 is a comparison of progression free survival after chemotherapy administered to high-risk integrative cluster groups (IntClust1, IntClust2, IntClust6, and IntClust9) and to lower risk groups (averaged together). The data suggests that IntClust2 molecular subgroups benefit greatly from chemotherapy as their progression free survival probability is higher (adj. P=0.045), as compared to lower risk groups and the other high-risk groups.

FIG. 45 provides a comparison of progression free survival in the molecular subgroup IntClust1 with and without an mTOR antagonist treatment. Specifically, patients receiving the mTOR inhibitor everolimus had profoundly greater survival probability than patients that did not receive an mTOR antagonist (adj. P value=0.023). This result suggests that utilizing an mTOR antagonist to specifically target the oncogenic driver RPS6KB1 of this subgroup can increase the probability progression free survival.

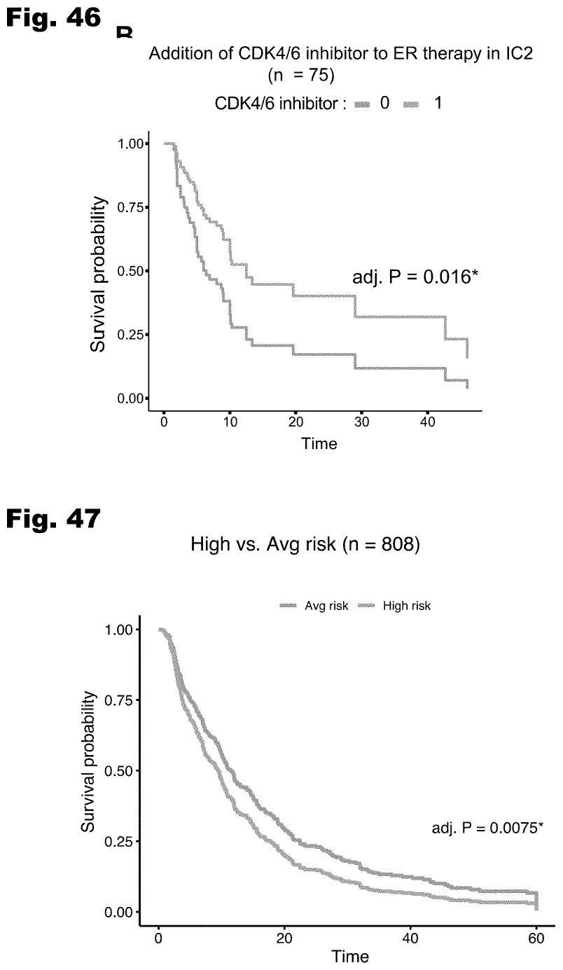

FIG. 46 provides a comparison of progression free survival in the molecular subgroup IntClust2 with and without an CDK4/6 antagonist treatment. Specifically, patients receiving a CDK4/6 inhibitor (palbociclib, ribociclib, or abemaciclib) had profoundly greater survival probability than patients that did not receive an CDK4/6 antagonist (adj. P value=0.016). This result suggests that utilizing an CDK4/6 antagonist to specifically target the oncogenic driver CDK4/6 of this subgroup can increase the probability progression free survival.

FIG. 47 provides a comparison of progression free survival after endocrine therapy (fulvestrant or tamoxifen) administered to high-risk integrative cluster groups (averaged together) and to lower risk groups (averaged together). The data suggests that lower risk groups have higher probability of progression free survival than high-risk groups (adj. P=0.0075).

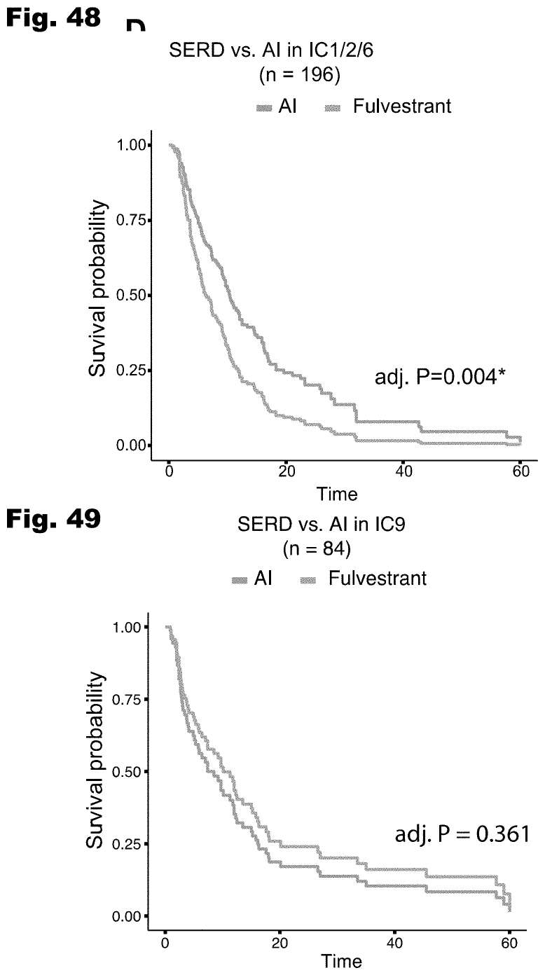

FIG. 48 provides a comparison of progression free survival in the molecular subgroups IntClust1, InClust2, and IntClust6 (averaged together) with aromatase inhibitor treatment and with selective estrogen receptor degrader (SERD) fulvestrant treatment. IntClust1, InClust2, and IntClust6 patients receiving an aromatase inhibitor had greater survival probability than patients receiving fulvestrant (adj. P value=0.004). This result suggests that an endocrine therapy utilizing an aromatase inhibitor can increase the probability progression free survival in patients within IntClust1, InClust2, and IntClust6.

FIG. 49 provides a comparison of progression free survival in the molecular subgroup IntClust9 with aromatase inhibitor treatment and with selective estrogen receptor degrader (SERD) fulvestrant treatment. IntClust9 patients receiving fulvestrant had slightly, yet insignificantly, greater survival probability than patients receiving an aromatase inhibitor (adj. P value=0.361). This result suggests that an endocrine therapy utilizing an aromatase inhibitor does not increase progression free survival in IntClust9, unlike IntClust1, InClust2, and IntClust6. Thus, endocrine treatments to various high-risk molecular subgroups should be tailored accordingly.

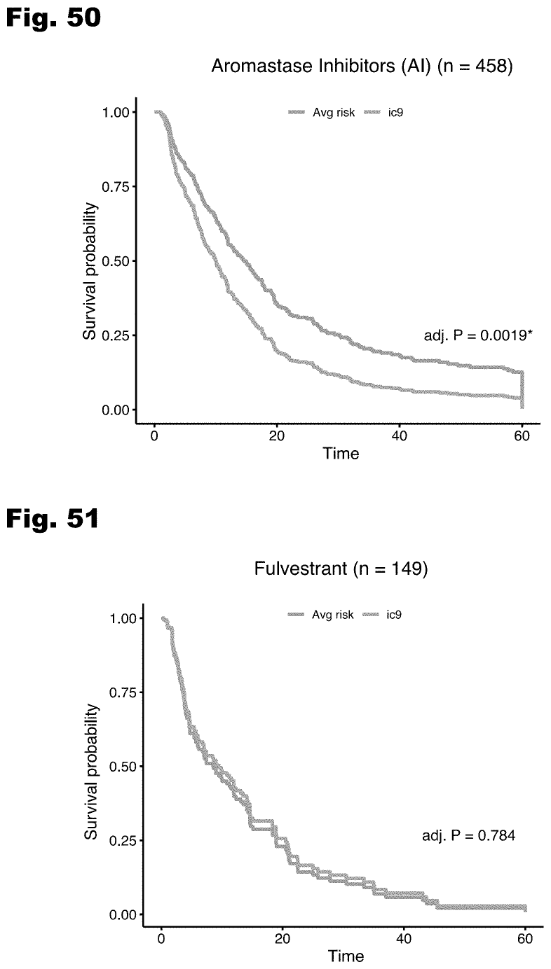

FIG. 50 provides a comparison of progression free survival after endocrine therapy utilizing an aromatase inhibitor administered to patients of the high-risk molecular group IntClust9 and to lower risk groups (averaged together). IntClust9 patients receiving an aromatase inhibitor had significantly less survival probability than lower risk patients receiving an aromatase inhibitor (adj. P value=0.0019). This result suggests that an endocrine therapy utilizing an aromatase inhibitor does not increase survival probability in the IntClust9 molecular group, but perhaps, instead a SERD or PROTAC ARV-471 might provide better results as these compounds mitigate estrogen-receptor signaling crosstalk.

FIG. 51 provides a comparison of progression free survival after endocrine therapy utilizing the SERD fulvestrant administered to patients of the high-risk molecular group IntClust9 and to lower risk groups (averaged together). IntClust9 patients receiving an fulvestrant had similar survival probability than lower risk patients receiving an fulvestrant (adj. P value=0.784). This result, combined with aromatase inhibitor result, suggests that an endocrine therapy utilizing a SERD provides a better survival probability in the IntClust9 molecular group than an endocrine therapy utilizing an aromatase inhibitor.

### Example 7: Patient Derived Organoids

Cancer patient derived organoids (PDOs) provide an ability to test various drugs on cancer cells in a preclinical setting. Within this example, breast cancer PDOs were developed, each patient PDO having a molecular pathology that falls within an integrated cluster molecular subgroup. The various developed PDOs were administered various drug compounds to determine their responsiveness. The results identify various candidate compounds to be evaluated in clinical trials for patients falling within a particular molecular subgroup. Or, alternatively, PDOs can be to clinical setting to identify particular drugs for a patient. In this scenario, cancer cells are extracted from the patient to yield PDOs to be treated with various drug compounds. Compounds with the best results can be utilized in a personalized therapy for the patient.

To assess breast cancer PDOs, the organoids were digested into single cells with TrypLE (Gibco). Cells are strained with a 100 μm filter then seeded as 10,000 cells per well with 10 μl beta-mercaptoethanol (BME) (Cultrex) in a black, clear bottom 96-well plate and covered with 100 μl breast organoid media. Cells are grown for 4 days to form small spheroids. Cells were treated with 6 concentrations of different targeted Therapies (including but not limited to capivasertib, ipatasertib, PF4706871, M2698, alpelisib), as well as negative control (DMSO) and positive control (Triton X-100) in duplicate for 8 days, with drug media refreshed on day 5. On day 8, the plates are manually checked under the microscope to ensure the positive control drug(s) had effectively killed organoids, and that organoids present in the negative control wells were healthy. Cell viability is assessed using AlamarBlue (Thermofisher) by adding the dye to the media in final concentration of 1:10, followed by incubation for 4 hours at 37° C., and luminescence measurement using a microplate reader (Molecular Devices). IC₅₀ values are computed using R package drc. Averages of IC₅₀s from two to three independent experiments were calculated and visualized using R.

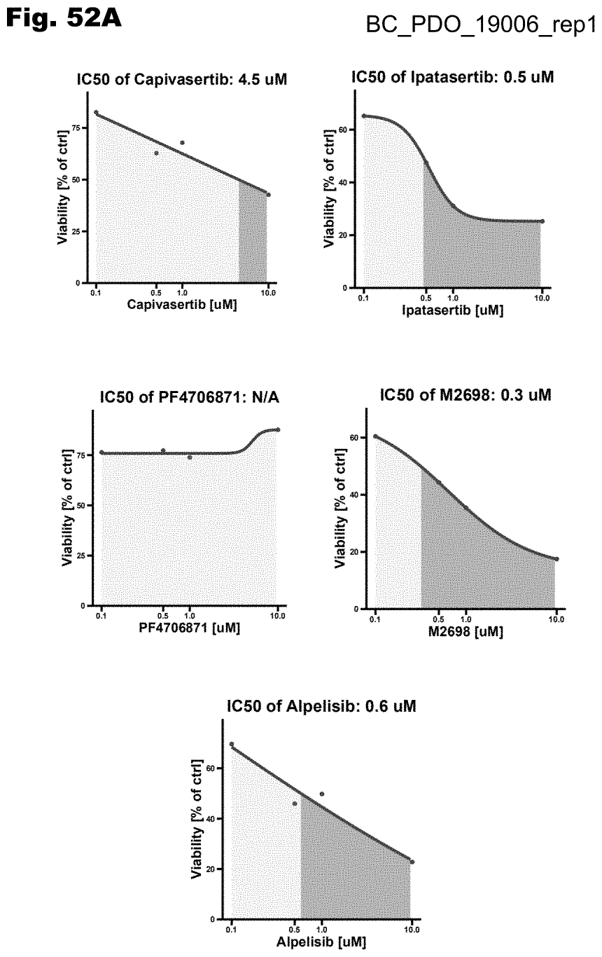

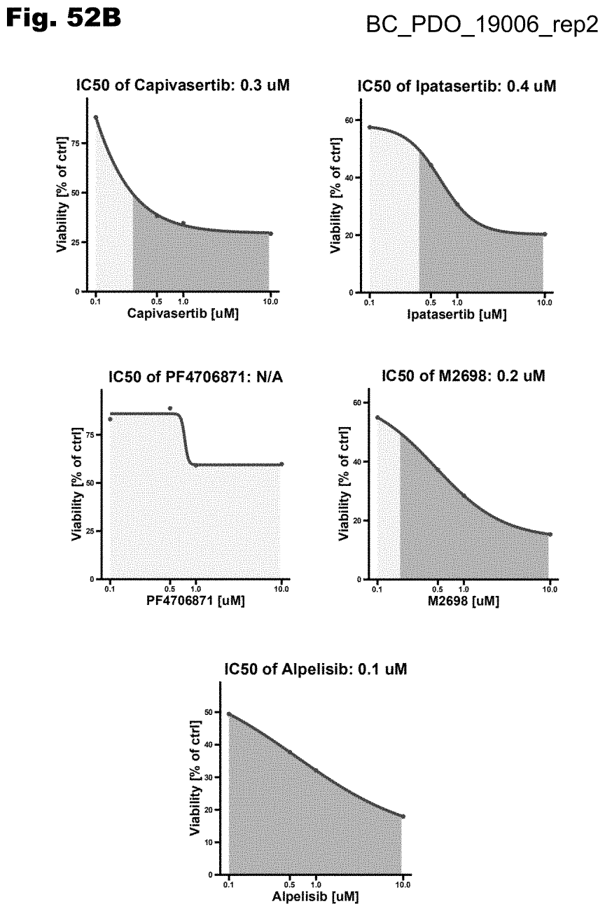

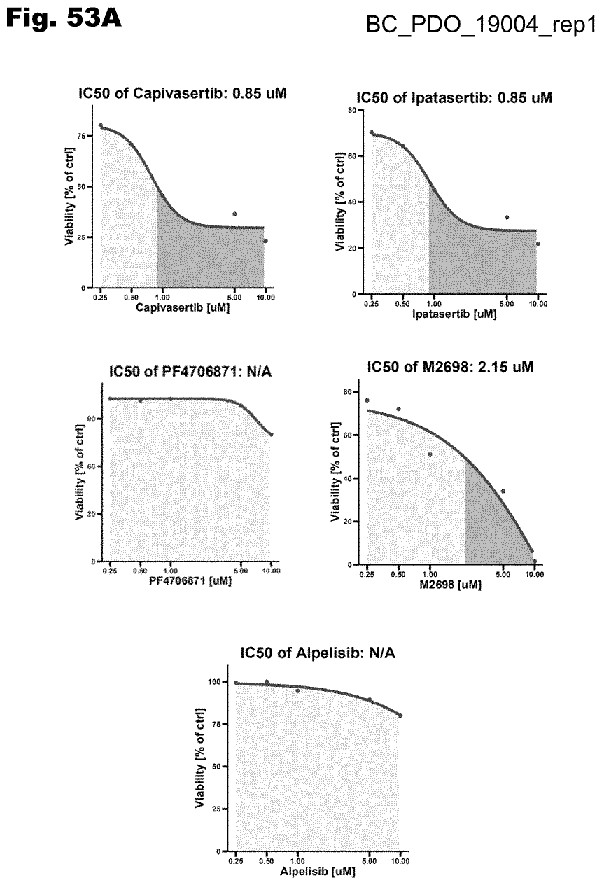

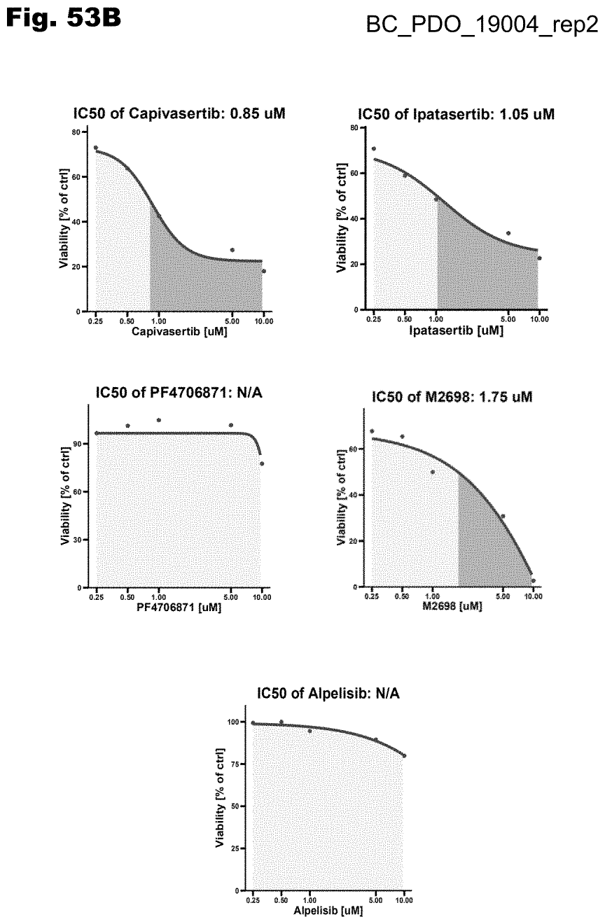

Exemplary results of ER-positive PDOs categorized in to IntClust4 are provided in FIGS. 52A to 53B. As can be seen, capivasertib, ipatasertib, M22698, and alpelisib, but not PF4706871, each provide IC₅₀ on the order of 100 nM to 10 μM for PDOs derived from the 19006 patient (FIGS. 52A and 52B). Likewise, capivasertib, ipatasertib, and M22698, but not alpelisib and PF4706871, each provide IC₅₀ on the order of 100 nM to 10 μM for PDOs derived from the 19006 patient (FIGS. 53A and 53B).

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method to treat an individual having breast cancer, comprising:
stratifying or having stratified, utilizing a risk stratification model, a breast cancer of an individual into a high risk of recurrence subgroup,
wherein the risk stratification model is a statistical model that incorporates features derived from integrative subtype clusters that are delineated by a molecular pathology; and
treating the individual to reduce the risk of recurrence by administering a prolonged treatment regimen that includes at least one of: chemotherapy, endocrine therapy, targeted therapy, or health professional surveillance.

2. The method of claim 1, wherein the risk stratification model utilizes one of: a multi-state semi-markov Model, a Cox Proportional Hazards model, a shrinkage based method, a tree based method, a Bayesian method, a kernel based method, or a neural network.

3. The method of claim 1, wherein the integrated subtype cluster features are:
membership to a given cluster or the posterior probability of membership to a given cluster.

4. The method of claim 1, wherein the integrative subtype clusters are determined by the IntClust classification model that incorporates molecular data as features.

5. The method of claim 4, wherein the molecular data is obtained by at least one of:
microarray based gene expression, microarray/SNP array based copy number inference, RNA-sequencing, targeted (capture) RNA-sequencing, exome sequencing, whole genome sequencing (WES/WGS), targeted (panel) sequencing, Nanostring nCounter for gene expression, Nanostring nCounter for copy number inference, Nanostring digital spatial profiler measurement of protein, Nanostring digital spatial profiler measurement of protein gene expression in situ, DNA-ISH, RNA-ISH, RNAScope, DNA Methylation assays, or ATAC-seq.

6. The method of claim 4, wherein the molecular data is derived utilizing a gene panel.

7. The method of claim 6, wherein the gene panel is one of: Foundation Medicine CDx, Memorial Sloan Kettering Cancer Center Integrated Mutation Profiling of Actionable Cancer Targets (MSK-IMPACT), Stanford Tumor Actionable Mutation Panel (STAMP), or UCSF500 Cancer Gene Panel.

8. The method of claim 1, wherein the risk stratification model utilizes at least one of:
clinical data, such as age, cancer stage, number of tumor positive lymph nodes, size of tumor, grade of tumor, surgery performed, treatment performed, or basic molecular identities.

9. The method of claim 1, wherein the risk stratification model utilizes the CTS5 algorithm.

10. The method of claim 1, wherein the risk stratification model incorporates one of:
Oncotype DX, Prosigna PAM50, Prosigna ROR, MammaPrint, EndoPredict or Breast Cancer Index (BC).

11. The method of claim 1, wherein the prolonged treatment regimen includes adjuvant chemotherapy.

12. The method of claim 1, wherein the prolonged treatment regimen includes treatment beyond the standard course of treatment.

13. A method to treat an individual having breast cancer, comprising:
stratifying or having stratified, utilizing a risk stratification model, a breast cancer of an individual into a lower risk of recurrence subgroup,
wherein the risk stratification model is a statistical model that incorporates features derived from integrative subtype clusters that are delineated by a molecular pathology; and
treating the individual to reduce the harmful effects of chemotherapy by administering a treatment regimen that includes surgery or endocrine therapy, but not chemotherapy.

14. The method of claim 13, wherein the risk stratification model utilizes one of: a multi-state semi-markov Model, a Cox Proportional Hazards model, a shrinkage based method, a tree based method, a Bayesian method, a kernel based method, or a neural network.

15. The method of claim 13, wherein the integrated subtype cluster features are:
membership to a given cluster or the posterior probability of membership to a given cluster.

16. The method of claim 13, wherein the integrative subtype clusters are determined by the IntClust classification model that incorporates molecular data as features.

17. The method of claim 16, wherein the molecular data is obtained by at least one of:
microarray based gene expression, microarray/SNP array based copy number inference, RNA-sequencing, targeted (capture) RNA-sequencing, exome sequencing, whole genome sequencing (WES/WGS), targeted (panel) sequencing, Nanostring nCounter for gene expression, Nanostring nCounter for copy number inference, Nanostring digital spatial profiler measurement of protein, Nanostring digital spatial profiler measurement of protein gene expression in situ, DNA-ISH, RNA-ISH, RNAScope, DNA Methylation assays, or ATAC-seq.

18. The method of claim 16, wherein the molecular data is derived utilizing a gene panel.

19. The method of claim 18, wherein the gene panel is one of: Foundation Medicine CDx, Memorial Sloan Kettering Cancer Center Integrated Mutation Profiling of Actionable Cancer Targets (MSK-IMPACT), Stanford Tumor Actionable Mutation Panel (STAMP), or UCSF500 Cancer Gene Panel.

20. The method of claim 13, wherein the risk stratification model utilizes at least one of: clinical data, such as age, cancer stage, number of tumor positive lymph nodes, size of tumor, grade of tumor, surgery performed, treatment performed, or basic molecular identities.

21. The method of claim 13, wherein the risk stratification model utilizes the CTS5 algorithm.

22. The method of claim 13, wherein the risk stratification model incorporates one of:
Oncotype DX, Prosigna PAM50, Prosigna ROR, MammaPrint, EndoPredict or Breast Cancer Index (BC).

23. The method of claim 13, wherein the treatment regimen includes adjuvant endocrine therapy.

24. A method to treat an individual having breast cancer, comprising:
determining or having determined results of an assay that has classified an individual's breast cancer into an integrated cluster (IntClust) subgroup, wherein the results indicate that the breast cancer is classified into one of: IntClust1, IntClust2, IntClust6, or IntClust9, and
treating the individual with a prolonged treatment regimen that includes at least one of: chemotherapy, endocrine therapy, targeted therapy, and health professional surveillance.

25. The method of claim 24, wherein the classification of the individual's breast cancer is performed utilizing a molecular class prediction tool.

26. The method of claim 25, wherein the molecular class prediction tool utilizes a shrinkage based method, logistic regression, a support vector machine with a linear kernel, a support vector machine with a gaussian kernel, or a neural network.

27. The method of claim 25, wherein the molecular class prediction tool incorporates molecular data as features.

28. The method of claim 27, wherein the molecular data features are copy number features, gene expression features, genomic methylation features, or occupancy features derived from DNA or RNA analysis of the individual's breast cancer.

29. The method of claim 27, wherein the molecular data is obtained by microarray based gene expression, microarray/SNP array based copy number inference, RNA-sequencing, targeted (capture) RNA-sequencing, exome sequencing, whole genome sequencing (WES/WGS), targeted (panel) sequencing, Nanostring nCounter for gene expression, Nanostring nCounter for copy number inference, Nanostring digital spatial profiler measurement of protein, Nanostring digital spatial profiler measurement of protein gene expression in situ, DNA-ISH, RNA-ISH, RNAScope, DNA Methylation assays, or ATAC-seq.

30. The method of claim 27, wherein the molecular data is derived utilizing a gene panel.

31. The method of claim 30, wherein the gene panel is Foundation Medicine CDx, Memorial Sloan Kettering Cancer Center Integrated Mutation Profiling of Actionable Cancer Targets (MSK-IMPACT), Stanford Tumor Actionable Mutation Panel (STAMP), or UCSF500 Cancer Gene Panel.

32. The method of claim 24, wherein the breast cancer the individual is administered adjuvant chemotherapy.

33. The method of claim 24, wherein the breast cancer the individual is administered extended endocrine therapy.

34. The method of claim 33, wherein the endocrine therapy comprises administering a selective estrogen receptor modulator, a selective estrogen receptor degrader, an aromatase inhibitor, or PROTAC ARV-471.

35. The method of claim 34, wherein the selective estrogen receptor modulator is tamoxifen, toremifene, raloxifene, ospemifene, or bazedoxifene.

36. The method of claim 34, wherein the selective estrogen receptor degrader is fulvestrant, brilanestrant (GDC-0810), elacestrant, GDC-9545, SAR439859 (SERD '859), RG6171, or AZD9833.

37. The method of claim 34, wherein the aromatase inhibitor is anastrozole, exemestane, letrozole, vorozole, formestane, or fadrozole.

38. The method of claim 24, wherein the breast cancer is classified into IntClust1 and the individual is administered an mTOR pathway antagonist, an AKT1 antagonist, an AKT1/RPS6KB1 antagonist, an RPS6KB1 antagonist, a PI3K antagonist, an elF4A antagonist, or an elF4E antagonist.

39. The method of claim 24, wherein the breast cancer is classified into IntClust2 and the individual is administered a CDK4/6 antagonist, an FGFR pathway antagonist, a PARP antagonist, a homologous recombination deficiency (HRD) targeted therapy, a PAK1 antagonist, an elF4A antagonist, or elF4E antagonist.

40. The method of claim 24, wherein the breast cancer is classified into IntClust6 and the individual is administered an FGFR pathway antagonist, an elF4A antagonists, or an elF4E antagonist.

41. The method of claim 24, wherein the breast cancer is classified into IntClust9 and the individual is administered a selective estrogen receptor degrader, an SRC3 antagonist, a MYC antagonist, a BET bromodomain antagonist, an elF4A antagonist, or an elF4E antagonist.

42. A method of treating an individual having breast cancer, comprising:
classifying or having classified an oncogenic pathology of an individual's cancer, wherein the oncogenic pathology indicates mTOR pathway;
administering to the individual an mTOR antagonist.

43. The method of claim 42, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

44. The method of claim 42, wherein the mTOR antagonist is everolimus, temsirolimus, sirolimus, or rapamycin.

45. A method of treating an individual having breast cancer, comprising:
classifying or having classified an oncogenic pathology of an individual's cancer, wherein the oncogenic pathology indicates AKT1;
administering to the individual an AKT1 antagonist.

46. The method of claim 45, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

47. The method of claim 45, wherein the AKT1 antagonist is ipatasertib, or capivasertib (AZD5363).

48. A method of treating an individual having breast cancer, comprising:
classifying or having classified an oncogenic pathology of an individual's cancer, wherein the oncogenic pathology indicates AKT1/RPS6KB1;
administering to the individual an AKT1/RPS6KB1 antagonist.

49. The method of claim 48, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

50. The method of claim 48, wherein the AKT1/RPS6KB1antagonist is M2698.

51. A method of treating an individual having breast cancer, comprising:
classifying or having classified an oncogenic pathology of an individual's cancer, wherein the oncogenic pathology indicates RPS6KB1;
administering to the individual an RPS6KB1 antagonist.

52. The method of claim 51, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

53. The method of claim 51, wherein the RPS6KB1 antagonist is LY2584702.

54. A method of treating an individual having breast cancer, comprising:
classifying or having classified an oncogenic pathology of an individual's cancer, wherein the oncogenic pathology indicates PI3K;
administering to the individual an PI3K antagonist.

55. The method of claim 54, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

56. The method of claim 54, wherein the PI3K antagonist is alpelisib, buparlisib (BKM120), or pictilisib (GDC-0941).

57. A method of treating an individual having breast cancer, comprising:
classifying or having classified an oncogenic pathology of an individual's cancer, wherein the oncogenic pathology indicates CDK4/6;
administering to the individual an CDK4/6 antagonist.

58. The method of claim 57, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

59. The method of claim 57, wherein the CDK4/6 antagonist is palbociclib, ribociclib, or abemaciclib.

60. A method of treating an individual having breast cancer, comprising:
classifying or having classified an oncogenic pathology of an individual's cancer, wherein the oncogenic pathology indicates FGFR pathway;
administering to the individual an FGFR pathway antagonist.

61. The method of claim 60, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

62. The method of claim 60, wherein the FGFR pathway antagonist is lucitanib, dovitinib, AZD4547, erdafitinib, infigratinib (BGJ398), BAY-1163877, or ponatinib.

63. A method of treating an individual having breast cancer, comprising:
classifying or having classified an oncogenic pathology of an individual's cancer, wherein the oncogenic pathology indicates SRC3;
administering to the individual an SRC3 antagonist.

64. The method of claim 63, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

65. The method of claim 63, wherein the SRC3 antagonist is Sl-2.

66. A method of treating an individual having breast cancer, comprising:
classifying or having classified an oncogenic pathology of an individual's cancer, wherein the oncogenic pathology indicates MYC;
administering to the individual a MYC antagonist.

67. The method of claim 66, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

68. The method of claim 66, wherein the MYC antagonist is omomyc.

69. A method of treating an individual having breast cancer, comprising:
classifying or having classified an oncogenic pathology of an individual's cancer, wherein the oncogenic pathology indicates BET bromodomain;
administering to the individual an BET bromodomain antagonist.

70. The method of claim 69, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

71. The method of claim 69, wherein the BET bromodomain antagonist is JQ1 or PROTAC ARV-771.

72. A method of treating an individual having breast cancer, comprising:
classifying or having classified an oncogenic pathology of an individual's cancer, wherein the oncogenic pathology indicates elF4A;
administering to the individual an elF4A antagonist.

73. The method of claim 72, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

74. The method of claim 72, wherein the elF4A antagonist is zotatifin.

75. A method of treating an individual having breast cancer, comprising:
classifying or having classified an oncogenic pathology of an individual's cancer, wherein the oncogenic pathology indicates elF4E;
administering to the individual an elF4E antagonist.

76. The method of claim 75, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

77. The method of claim 75, wherein the elF4E antagonist is rapamycin, a rapamycin analogue, ribavirin, or AZD8055.

78. A method of treating an individual having breast cancer, comprising:
classifying or having classified an oncogenic pathology of an individual's cancer, wherein the oncogenic pathology indicates PARP;
administering to the individual a PARP antagonist.

79. The method of claim 78, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

80. The method of claim 78, wherein the PARP antagonist is niraparib or olaparib.

81. A method of treating an individual having breast cancer, comprising:
classifying or having classified an oncogenic pathology of an individual's cancer, wherein the oncogenic pathology indicates PAK1;
administering to the individual a PAK1 antagonist.

82. The method of claim 81, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the individual's breast cancer.

83. The method of claim 81, wherein the PAK1 antagonist is IPA3.

84. A method to assess drug compounds utilizing breast cancer patient derived organoids, comprising:
extracting cancer cells from one or more patients;
classifying the oncogenic pathology of each patient's cancer into a molecular pathology subgroup;
developing a panel of patient derived organoid lines utilizing the extracted cancer cells, wherein each patient derived organoid line of the panel is within the same molecular pathology subgroup; and
administering a plurality of drug compounds on the panel of patient derived organoid lines to assess the toxicity of each drug compound.

85. The method of claim 84, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the patient's breast cancer or of the patient derived organoid line.

86. The method of claim 84, wherein the molecular pathology subgroup is an integrated cluster subgroup.

87. The method of claim 84, wherein compound concentration is assessed.

88. The method of claim 84, wherein compound toxicity on healthy cells is assessed.

89. A method to assess drug compounds for a personalized treatment utilizing breast cancer patient derived organoids, comprising:
extracting cancer cells from a patient;
classifying the oncogenic pathology the patient's cancer into a molecular pathology subgroup;
developing a one or more patient derived organoid lines using the extracted cancer cells; and
administering a plurality of drug compounds on the one or more patient derived organoid lines to assess the toxicity of each drug compound, wherein the drug compounds to be administered are candidate compounds associated with the molecular pathology subgroup.

90. The method of claim 89, wherein the oncogenic pathology is classified utilizing a molecular class prediction tool that utilizes:
a shrinkage based method, logistic regression, a support vector machine with a linear kernel, a support vector machine with a gaussian kernel, or a neural network; and
copy number features, gene expression features, genomic methylation features, or nucleosome occupancy features derived from DNA or RNA analysis of the patient's breast cancer or of the patient derived organoid line.

91. The method of claim 89, wherein the molecular pathology subgroup is an integrated cluster subgroup.

92. The method of claim 89, wherein compound concentration is assessed.

93. The method of claim 89, wherein compound toxicity on healthy cells is assessed.

94. The method of claim 89, wherein at least one combination of the drug compounds is assessed.

95. The method of claim 89 further comprising:
administering to the patient a drug compound of the plurality of drug compounds based on the drug compound's toxicity on the one or more patient derived organoid lines.

96. The method of claim 95, wherein the drug compound is administered as an adjuvant therapy.
